(19) United States
(12) Patent Application Publication
(10) Pub. No.: US 2025/0275968 A1
(43) Pub. Date: Sep. 4, 2025

(54) INHIBITORS OF TAU PROTEINS

(71) Applicant: DEUTSCHES ZENTRUM FÜR NEURODEGENERATIVE ERKRANKUNGEN E.V., Bonn (DE)

(72) Inventors: Markus ZWECKSTETTER, Göttingen (DE); Lisa-Marie RAMIREZ, Göttingen (DE); Alain IBANEZ DE OPAKUA LOPEZ DE ABETXUKO, Göttingen (DE); Ina VORBERG, Bonn (DE); Alina HEBESTREIT, Bonn (DE)

(73) Assignee: DEUTSCHES ZENTRUM FÜR NEURODEGENERATIVE ERKRANKUNGEN E.V., Bonn (DE)

(21) Appl. No.: 18/862,466

(22) PCT Filed: May 3, 2023
(86) PCT No.: PCT/EP2023/061621

§ 371 Date: Nov. 1, 2024

(30) Foreign Application Priority Data
May 3, 2022 (EP) 22171346.4

(51) Int. Cl. A61K 31/506; A61K 31/4439; A61K 31/4545; A61P 25/28; C07D 401/14; C07D 403/04; C07D 403/14; C07D 487/10

(52) U.S. Cl. CPC A61K 31/506; A61K 31/4439; A61K 31/4545; A61P 25/28; C07D 401/14; C07D 403/04; C07D 403/14; C07D 487/10

(57) ABSTRACT

The present invention relates to a compound and pharmaceutical compositions thereof for use in preventing or treating a tauopathy.

### TECHNICAL FIELD OF THE INVENTION

The present invention relates to a compound and pharmaceutical compositions thereof for use in preventing or treating a tauopathy.

### BACKGROUND ART

Tau is a microtubule associated protein mainly found in axons and dendrites of neurons, where it functions in microtubule organization and regulation. The accumulation of insoluble tau aggregates characterizes several neurodegenerative diseases, known as tauopathies, including for example Alzheimer's disease (AD), Pick's disease (PiD), Huntington's disease (HD), progressive supranuclear palsy (PSP), corticobasal degeneration (CBD), chronic traumatic encephalopathy (CTE), argyrophilic grain disease (AGD) and frontotemporal dementia with Parkinsonism-17 (FTDP-17).¹ The neuropathological hallmarks of AD are extracellular amyloid plaques composed of amyloid-β (Aβ), as well as intracellular inclusions of aggregated tau called neurofibrillary tangles (NFTs).²

The spread of aggregated tau pathology is linked to the degree of clinical dementia in AD.^{3,4} Tau pathology is thought to spread throughout the brain in a typical manner: aggregates of tau first form in the locus coereleus and spread via the entorhinal cortex, next to the hippocampus, and the neocortex.⁵ Thus potential therapeutic strategies against AD include targeting tau aggregation and spreading. Moreover, tau aggregation can be observed at least 20 years before the onset of AD clinical symptoms indicating that inhibiting this process may be a preventive measure against AD.^{6,7}

Pharmacologic agents developed to target amyloid plaque or tau aggregates in AD are classified as “disease-modifying therapies (DMTs)” against AD, following the terminology presented by the Common Alzheimer's and Related Dementias Research Ontology (CADRO).⁸ Among the DMTs in the AD drug development pipeline in the past ˜5 years, Aβ-directed therapies represented the majority, while tau-based therapies fell behind.⁹ Notable examples of Aβ-targeting DMTs are Aducanumab and Lecanemab, which were granted approval by the U.S. Federal Drug Authority (FDA) in 2021 and 2023, respectively. However, some AD-patients treated with these two antibodies experienced brain bleeding, pointing to a potentially poor risk-to-benefit ratio. Numerous clinical trials for other AB-directed drugs have failed, mostly due to lack of efficacy.⁶ In the case of tau-based DMTs, progress was made in the identification of small molecules that disrupt tau aggregation. However, to date, there are no tau-based DMTs that have achieved FDA approval.

Thus, there is a need for drugs that allow the prevention or treatment of a tauopathy.

### SUMMARY OF THE INVENTION

The invention is directed to a compound according to formula (I) for use in preventing or treating a tauopathy,

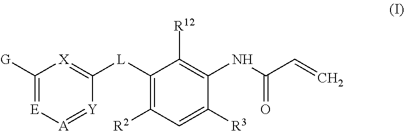

wherein
G is

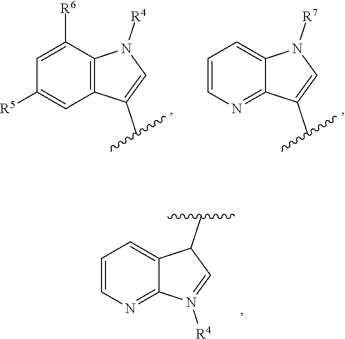

preferably

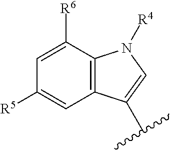

X is N or C—H, preferably N;
Y is N or C—H, preferably N;
L is

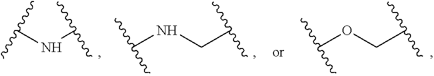

preferably

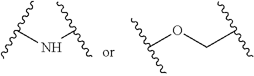

A is N or C—R; preferably C—R, more preferably CH
E is N or C—R¹; preferably C—R¹, more preferably CH
R is hydrogen, fluoro, chloro, bromo, —(C₁-C₆)alkyl, cyano, or —O(C₁-C₆)alkyl, preferably hydrogen;
R¹ is hydrogen, fluoro, chloro, bromo, —(C₁-C₆)alkyl, cyano, or —O(C₁-C₆)alkyl, preferably hydrogen;
R² is hydrogen, fluoro, chloro, bromo, —(C₁-C₆)alkyl, cyano, —O(C₁-C₆)alkyl, or difluoromethoxy, preferably —O(C₁-C₆)alkyl;
R³ is

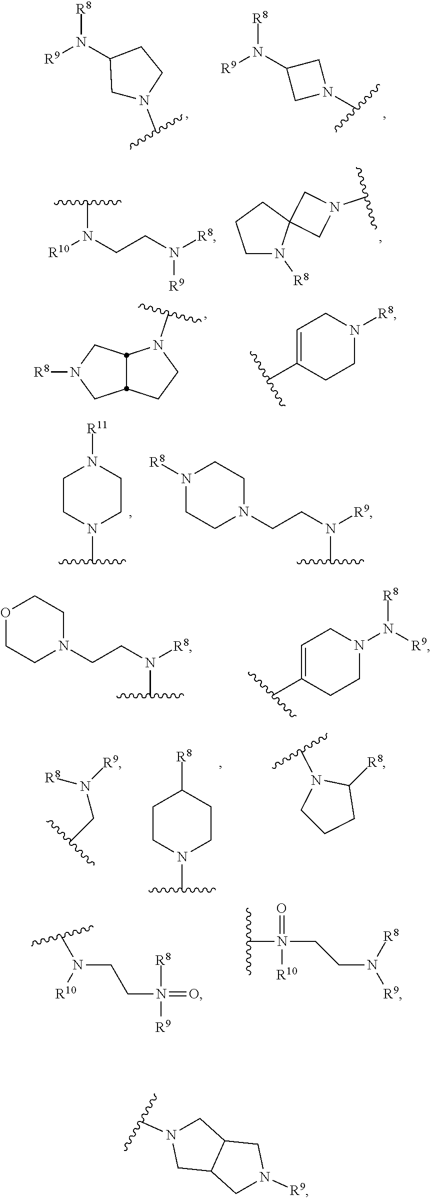

or —NR⁸R⁹, preferably

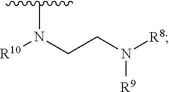

R⁴ is hydrogen, —(C₁-C₆)alkyl, or —(C₃-C₆)cycloalkyl;
R⁵ is hydrogen, fluoro, or chloro;
R⁶ is hydrogen, fluoro or chloro;
R⁷ is hydrogen, or —(C₁-C₆)alkyl;
R³ is hydrogen, or —(C₁-C₆)alkyl;
R⁹ is hydrogen, or —(C₁-C₆)alkyl;
R¹⁰ is hydrogen, or —(C₁-C₆)alkyl;
R¹¹ is hydrogen, —(C₁-C₆)alkyl, or —CH₂CON((C₁-C₆)alkyl)₂;
R¹² is hydrogen, or —(C₁-C₆)alkyl;
wherein optionally,
one or more hydrogen atoms may be replaced by a deuterium atom;
one or more carbon atoms may be replaced by the corresponding ¹¹C isotope;
one or more nitrogen atoms may be replaced by the corresponding ¹³N-isotope;
one or more fluoro atoms may be replaced by the corresponding ¹⁸F-isotope;
or a pharmaceutically acceptable salt thereof.

In a further embodiment, the invention is directed to a compound according to formula (I)

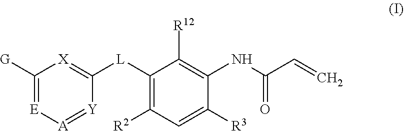

wherein
G is

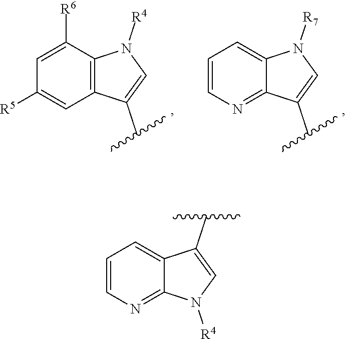

preferably

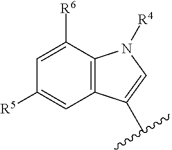

X is N or C—H, preferably N;
Y is N or C—H, preferably N;
L is

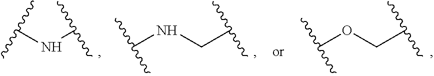

preferably

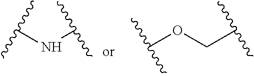

A is N or C—R; preferably C—R, more preferably CH
E is N or C—R¹; preferably C—R¹, more preferably CH
R is hydrogen, fluoro, chloro, bromo, —(C₁-C₆)alkyl, cyano, or —O(C₁-C₆)alkyl, preferably hydrogen;
R¹ is hydrogen, fluoro, chloro, bromo, —(C₁-C₆)alkyl, cyano, or —O(C₁-C₆)alkyl, preferably hydrogen;
R² is hydrogen, fluoro, chloro, bromo, —(C₁-C₆)alkyl, cyano, —O(C₁-C₆)alkyl, or difluoromethoxy; preferably —O(C₁-C₆)alkyl;
R³ is

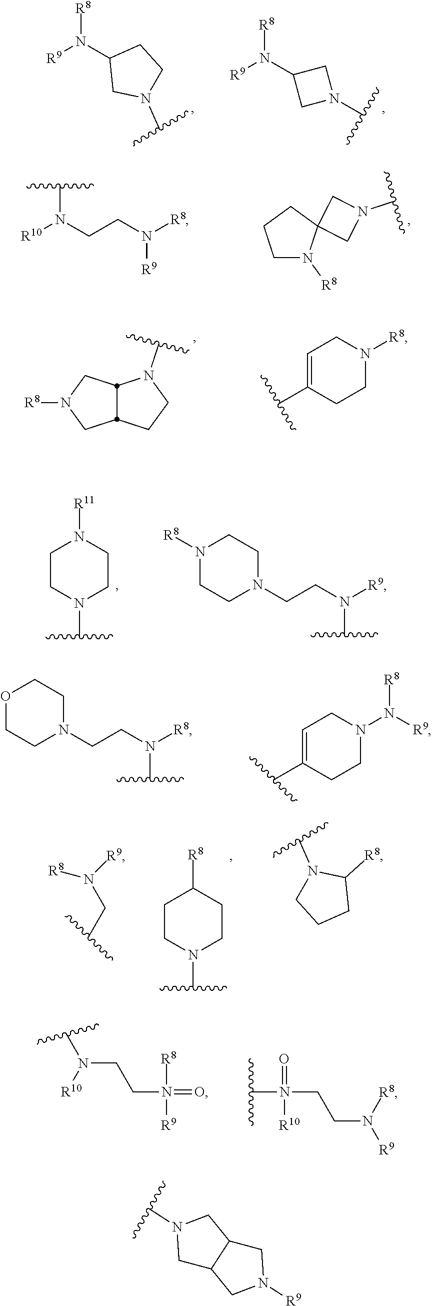

or —NR⁸R⁹, preferably

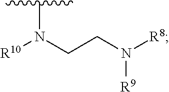

R⁴ is hydrogen, —(C₁-C₆)alkyl, or —(C₃-C₆)cycloalkyl;
R⁵ is hydrogen, fluoro, or chloro;
R⁶ is hydrogen, fluoro or chloro;
R⁷ is hydrogen, or —(C₁-C₆)alkyl;
R³ is hydrogen, or —(C₁-C₆)alkyl, preferably —(C₁-C₆)alkyl;
R⁹ is hydrogen, or —(C₁-C₆)alkyl, preferably —(C₁-C₆)alkyl;
R¹⁰ is hydrogen, or —(C₁-C₆)alkyl;
R¹¹ is hydrogen, —(C₁-C₆)alkyl, or —CH₂CON((C₁-C₆)alkyl)l;
R¹² is hydrogen, or —(C₁-C₆)alkyl;
with the provision that the compound is not selected from the group consisting of

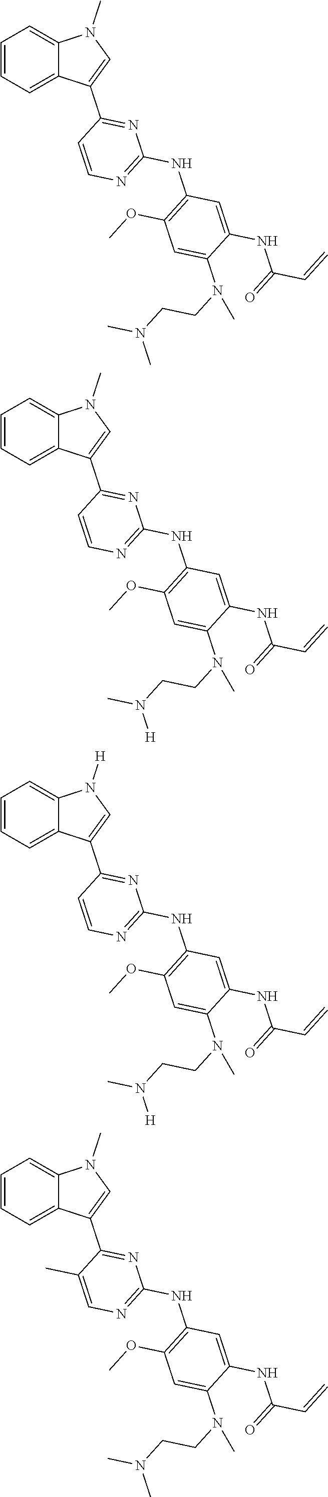

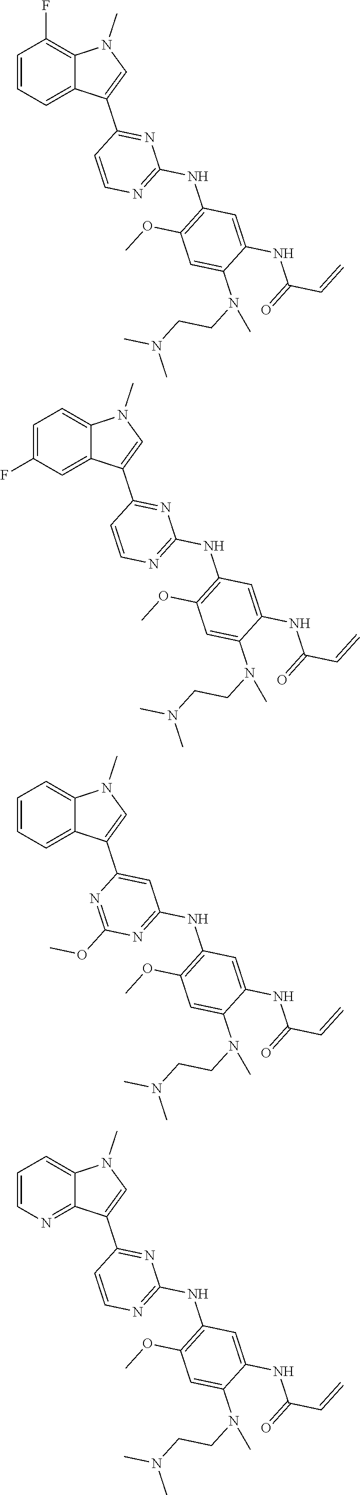

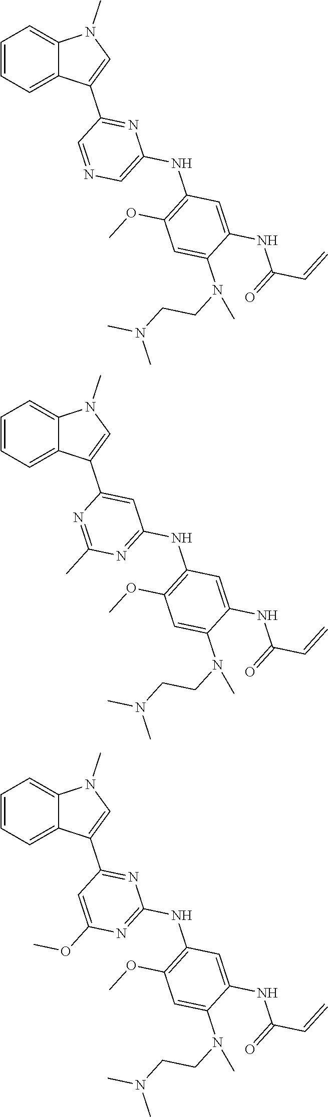

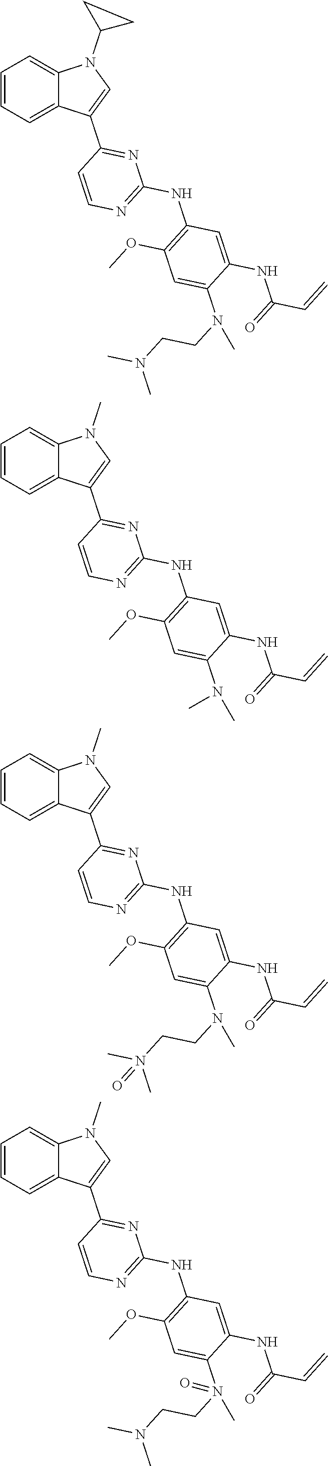

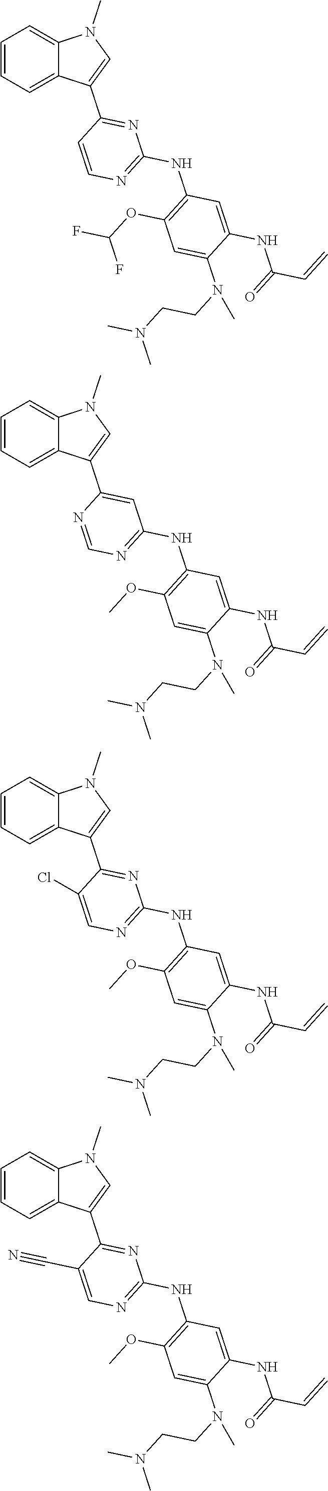 [SMILES: C=CC(=O)Nc1cc(Nc2nccc(-c3cn(C)c4ccccc34)n2)c(OC(F)F)cc1N(C)CCN(C)C ; C=CC(=O)Nc1cc(Nc2cc(-c3cn(C)c4ccccc34)ncn2)c(OC)cc1N(C)CCN(C)C ; C=CC(=O)Nc1cc(Nc2ncc(Cl)c(-c3cn(C)c4ccccc34)n2)c(OC)cc1N(C)CCN(C)C ; C=CC(=O)Nc1cc(Nc2ncc(C#N)c(-c3cn(C)c4ccccc34)n2)c(OC)cc1N(C)CCN(C)C]

wherein optionally,
one or more hydrogen atoms may be replaced by a deuterium atom;
one or more carbon atoms may be replaced by the corresponding ¹¹C isotope;
one or more nitrogen atoms may be replaced by the corresponding ¹³N isotope;
one or more fluoro atoms may be replaced by the corresponding ¹⁸F isotope;
or a pharmaceutically acceptable salt thereof.

The inventive compounds have been found to covalently bind to Cys291 and Cys322 of tau and inhibit tau aggregation in vitro. It has been further demonstrated that the compounds disrupts tau aggregation in situ in cells, and tau-induced pathology in vivo in a tau-overexpressing Drosophila model (see FIG. 12).

In a further embodiment, the invention is directed to a pharmaceutical composition comprising the compound according to formula (I), (Ia) and/or (Ib) and at least one pharmaceutically acceptable carrier for use in preventing or treating a tauopathy.

The compound or the pharmaceutical composition of the present invention is further for use in the prevention or treatment of a tauopathy selected from the group consisting of Alzheimer's disease, frontotemporal dementia, primary age-related tauopathy (PART), familial British dementia (FBD), familial Danish dementia (FDD), chronic traumatic encephalopathy (CTE), progressive supranuclear palsy (PSP), corticobasal degeneration (CBD), Pick's disease (PiD), dementia with Lewy Bodies (DLB), progressive supranuclear palsy (PSP), globular glial tauopathy (GGT), tauopathy with hippocampal 4-repeat tau immunoreactive spherical inclusions, limbic-predominant neuronal inclusion body 4R tauopathy (LNT), frontotemporal dementia and parkinsonism linked to chromosome 17 (FTDP-17), argyrophilic grain disease (AGD), Huntington disease, glial globular tauopathy, neuro-astroglial tauopathy variants in the elderly, familial behavioural variant frontotemporal dementia associated with astrocyte-predominant tauopathy, amyotrophic lateral sclerosis (ALS), spinocerebellar ataxia type 11, spinal muscular atrophy (SMA), progressive ataxia and palatal tremor-related tauopathy, cerebral amyloid angiopathy (CAA), IgLON5 antibody-related tauopathy, Chromosome 21 trisomy-related Alzheimer's disease (Down's syndrome), vascular dementia, cerebral amyloid angiopathy, Gerstmann-Straussler-Scheinker disease (GSS), Creutzfeldt-Jakob disease, fatal familial insomnia, Kuru, Niemann-Pick disease type C-related tauopathy, Nodding syndrome, Non-Guamanian motor neuron disease with neurofibrillary tangles, Parkinson's disease (PD), Parkinson's disease with dementia, Parkinsonism-dementia of Guam, Guadeloupean parkinsonism, Kosaka-Shibayama disease, post-encephalitic parkinsonism, SYNJ1 (PARK20) early-onset recessive form of parkinsonism with seizures and dystonia associated nigral tau pathology, multiple system atrophy, tau pathology associated with familial parkinsonism and progressive respiratory failure, limbic predominant neuronal-glial tau pathology in TARDBP gene mutations (1383; P112H), neuronal 4R tau pathology in fatal familial insomnia (PRNP D178N mutation), tau pathology associated with ADCY5 dyskinesia, tau pathology in chronic temporal lobe epilepsy, neurodegeneration with brain iron accumulation (NBIA), tau pathology in NBIA PANK2 and WDR45 gene mutations, tau pathology in NBIA PLA2G6 mutation, tau pathology in NBIA associated with autosomal-dominant mitochondrial membrane protein-associated neurodegeneration (MPAN), neuropil threads pretangles and neurofibrillary tangles in human immunodeficiency virus (HIV)-negative opiate abusers, tau pathology associated with acquired immunodeficiency syndrome (AIDS), diffuse neurofibrillary tangles with calcification, progressive ataxia and palatal tremor, SLC9A6-related parkinsonism, tau pathology associated with SPG7 gene mutation, striatal 4R tau pathology associated to X-linked parkinsonism with spasticity (ATP6AP2), tau pathology associated with SPAST gene-related hereditary spastic paraplegia, autism, autism spectrum disorders, retinal tauopathy, West Nile encephalomyelitis, TTBK2 gene-related spinocerebellar ataxia 11, herpes simplex encephalitis, tangle-only dementia (TOD), aging-related tau astrogliopathy (ARTAG), hippocampal tauopathy, subacute sclerosing panencephalitis (SSPE)-related tauopathy, FTLD-C9ORF72, Christianson syndrome, vacuolar tauopathy, lytico-bodig disease, ganglioglioma and gangliocytoma, meningioangiomatosis, lead encephalopathy, tuberous sclerosis complex, pantothenate kinase-associated neurodegeneration, neuronal ceroid lipofuscinosis, myotonic dystrophy, Fukuyama congenital muscular dystrophy, hemimegalencephaly, focal cortical dysplasias, Wolcott-Rallison syndrome, primary lateral sclerosis, progressive gait freezing, PSP with parkinsonism, Richardson's syndrome, non-fluent/agrammatic variant of primary progressive aphasia, semantic variant of primary progressive aphasia, logopenic variant of primary progressive aphasia, primary progressive apraxia of speech, amnestic Alzheimer's disease, preferably Alzheimer's disease (AD), Pick's disease (PiD), progressive supranuclear palsy (PSP), corticobasal degeneration (CBD), chronic traumatic encephalopathy (CTE), argyrophilic grain disease (AGD), frontotemporal dementia (FTD), amytrophic lateral sclerosis (ALS), Parkinson's disease (PD) and primary age-related tauopathy (PART).

### BRIEF DESCRIPTION OF THE FIGURES

FIG. 1: In vitro seeded aggregation/fibrillization of full-length tau is inhibited in a dose-dependent manner by Osimertinib, compound 2 (AZ7550) and compound 3 (AZ5104). (A) Effect of Osimertinib, compound 2 and 3 in various formulations on ThT fluorescence curves of 2N4R tau. Within 3 days the tau aggregation reached an equilibrium state, corresponding to maximal ThT fluorescence. At this point the aggregation mixture containing Osimertinib was pelleted and the amount of soluble tau in the mixture was quantified by sodium dodecyl sulfate polyacrylamide gel electrophoresis (SDS-PAGE). A representative SDS-PAGE gel is shown in B. The amount of soluble tau in the absence of Osimertinib (Lane 3) is about 80-90% lower than the control without fibrillization (Lane 2). (C) At a mole ratio of 1:10 (tau:Osimertinib), Osimertinib significantly increases the amount of soluble tau. Error bars correspond to the standard deviation of band intensities from two replicates. Statistically significant differences were determined by one-way ANOVA analysis with Dunnett's multiple comparison test, p<0.033(*).

FIG. 2: In vitro seeded fibrillization assay of 2N4R tau in the presence of Osimertinib and 2.5% (v/v) dimethylsulfoxide (DMSO). Osimertinib without cosolvents or counterions has poor solubility in water, which interferes with fluorescence-based aggregation assays. To circumvent this problem, aggregation assays and other experiments may be performed in the presence of the co-solvent DMSO. At a concentration of 2.5% (v/v) DMSO, a strong inhibitory activity of Osimertinib towards tau aggregation is present.

FIG. 3: Osimertinib mesylate more effectively inhibits the aggregation of wild-type tau (tau^{wt}) and 3R tau (tau^{3R}) than the aggregation of a cysteine-free tau mutant (tau^{C291S,C322S}). In vitro seeded aggregation/fibrillization was performed by incubating monomeric 2N4R tau^{wt}, tau^{3R} or 2N4R tau^{C291S,C322S} with Osimertinib mesylate (tau:compound mole ratios of 1:3 and 1:10) and the increase in ThT fluorescence was monitored over time (A). The normalized Tₘ values for 1:3 and 1:10 mole ratios do not show a clear dose-dependence (B). However, statistically lower normalized Span values were observed at 1:3 and 1:10 mole ratios for tau^{v} and tau^{3R} which are not observed in tau^{C291S,C322S} thus indicating the involvement of cysteine residues in binding to the compound (C). Statistically significant differences were determined by one-way unpaired t-test. Significant differences corresponding to p≤0.002(**), p≤0.0002(***), and p≤0.0001(****) are indicated.

FIG. 4: Osimertinib, compound 2 (AZ7550 hydrochloride), and compound 3 (AZ5104) partially dissolve 2N4R tau aggregates in vitro. (A) ThT fluorescence curves monitoring tau aggregation. tau fibrillization reached a saturation point within about 40 h, then compounds or buffer (Reference) were added to tau fibrils (tau monomer:Compound mole ratios 1:3 and 1:10). The experiment was performed in duplicate. (B) Addition of compounds resulted in the reduction of ThT fluorescence intensity which suggests the partial dissolution of tau aggregates. Statistically significant differences were determined by one-way ANOVA with Dunnett's multiple comparison test. Significant differences corresponding to p≤0.0002(***) and p≤0.0001(****) are indicated. (C) After 1 day of incubation with the compounds, transmission electron micrographs of the tau-compound mixtures show the presence of fibrils, indicating that the compounds do not fully dissolve fibrils. Scale bars are 500 nm. (D) SDS-PAGE analysis of pelleted tau-compound mixtures shows the increase in amount of soluble tau after incubation of tau fibrils with compound 3 (AZ5104) and compound 2 (AZ7550 hydrochloride (1:10 tau:Compound)). The dotted line corresponds to one standard deviation above the Reference value.

FIG. 5: NMR spectroscopy reveals binding of Osimertinib mesylate, compound 2 (AZ7550 hydrochloride), and compound 3 (AZ5104) to tau. ¹⁵N-labeled 2N4R tau (18 μM tau in 50 mM sodium phosphate pH 6.8) was incubated for 16 h at 37° C. in the presence or absence of compounds, then SOFAST-heteronuclear multiple quantum coherence (HMQC) spectra were recorded at 5° C. (A) Overlaid spectra of 2N4R tau with (black) and without (grey) a 100-fold molar excess of Osimertinib mesylate. (B) A zoomed-in version of the boxed region from (A) shows peaks from residues belonging to the N-terminal region of tau that have prominent broadening. Chemical shift perturbations (CSP) above ˜0.002 ppm as well as signal broadening (I/I₀<1) induced by the addition of compounds suggest binding of the compounds to the monomeric form of tau. I/I₀ values above 1, as seen in the case of compound 3 (AZ5104), suggest that the compound dissolves tau oligomers formed during the 16 h incubation period thus leading to increased NMR signal intensity.

FIG. 6: Electrospray ionization mass spectrometry (ESI-MS) confirms covalent modification of cysteines in tau(244-372, 13818 Da) by Osimertinib (500 Da), compound 2 (AZ7550) (486 Da) without the hydrochloride salt, and compound 3 (AZ5104) (486 Da).

FIG. 7: MS/MS analysis of the Osimertinib adduct in tau. Full-length 2N4R tau was incubated with Osimertinib mesylate (1:5 mole ratio of tau:compound) overnight at 37° C., subjected to tryptic digestion, and tryptic digests were analyzed by LC-MS/MS. Peptide mass spectra corresponding to Osimertinib-modified C291 (A) and C322 (B) confirm covalent modification of cysteines in tau. (C) Overall, about 350 tau peptide spectrum matches show Osimertinib-modified cysteine sites for the Osimertinib-treated tau sample. (D) The negative control corresponding to tau incubated at 37° C. without compound shows a small number of false positive peptide spectrum matches.

FIG. 8: Determination of the binding epitopes of Osimertinib to monomeric tau, compared against those of compound 2 (AZ7550 hydrochloride) and compound 3 (AZ5104). Addition of monomeric 2N4R tau (10 μM and 20 μM) to the compounds (250 μM in 50 mM sodium phosphate buffer with 2.5% v/v DMSO at pH 6.8, 37° C.) induced chemical shift perturbations (CSPs) in the 1D NMR spectra of the compounds, which allowed for the identification of aliphatic and aromatic protons involved in binding with monomeric tau (A-B). CSPs were ranked and mapped onto the chemical structure of Osimertinib (C). The large CSPs represent the most likely binding epitopes of Osimertinib.

FIG. 9: Determination of tau fibril binding epitopes of Osimertinib by saturation transfer difference (STD) NMR spectroscopy. Tau fibrils were incubated with a 48-fold excess of Osimertinib (A), compound 2 (AZ7550 hydrochloride) (B), or compound 3 (AZ5104) (C) in 50 mM sodium phosphate with 100 mM NaCl and 2.5% (v/v) DMSO at pH 6.8, 37° C. 1D ¹H NMR STD spectra were acquired in an interleaved manner, alternating between off-resonance irradiation at 60 ppm and on-resonance irradiation at −2 ppm. The control 1D spectra are shown in grey, and the difference spectra are shown in black. (D-F) The intensities of the STD effect (I_{STD}) were measured and normalized with respect to the largest STD signal (indole methyl protons set as 100%). (G-1) Protons showing STD effects are encircled in the chemical structures of Osimertinib, compound 2 (AZ7550 hydrochloride), and compound 3 (AZ5104). These protons represent tau fibril binding epitopes of each compound.

FIG. 10: Osimertinib inhibits the seeded aggregation of tau in HEK biosensor cells. Aggregate formation in cells was induced by lysate from HEK cells containing tau aggregates seeded by tau^{P301L} fibrils. Cells were incubated with the drug (0.25 μM or 7.5 μM) for 22 hours in the presence of lipofectamine. Cells incubated with a higher concentration of Osimertinib showed decreased aggregate formation.

FIG. 11: Comparison between inhibitory activities of Osimertinib, compound 2 (AZ7550) hydrochloride, and compound 3 (AZ5104) toward the aggregation of tau in HEK biosensor cells. The cell assay was performed using different batches of HEK cells (each dose response curve corresponds to one batch of cells) as described above (see FIG. 10) and the number of cells with aggregates (grey) was quantified relative to the negative control represented by cells treated with the vehicle (DMSO). The total amount of cells (black) remained close to 100% up until a compound concentration of about 5.0 μM, indicating low toxicity within the concentration range 0-5.0 μM. The half-maximal inhibitory concentration (IC₅₀) was determined by curve fitting. Control compounds such as Rociletinib and EGCG showed much lower inhibitory activity.

FIG. 12: Osimertinib mesylate and compound 2 (AZ7550) mesylate rescue tau-induced degeneration of Drosophila eyes. (A-C) Flies with (GMR-Gal4/WT tau) and without (GMR-Gal4/+) tau expression were treated with Osimertinib mesylate or compound 2 (AZ7550) mesylate from the larvae stage up to the adult stage. Percent degeneration was assessed from microscope images of Drosophila eyes. For both Osimertinib mesylate and compound 2 (AZ7550) mesylate five independent replicates with different flies on different days were performed. Statistically different groups were determined by unpaired t-test with p≤0.05(*), p≤0.01(**), p≤0.001(***), p≤0.0001(****). (D) Representative SEM micrographs of Drosophila treated with Osimertinib show visibly reduced signs of degeneration compared to the control with GMR-Gal4/WT tau.

FIG. 13: Comparison of 2N4R Tau aggregation inhibition activity of exemplar compounds against Compound 1 (Osimertinib). In vitro seeded fibrillization assay of 2N4R tau was performed in the absence (Reference) or presence of exemplar compounds, using 1:3 Tau:compound mole ratios and 2.5% (v/v) dimethylsulfoxide (DMSO) with at least 3 replicates. The midpoint of the aggregation curve, Tₘ, was quantified and normalized for each condition and is a measure of the extent of aggregation delay. Among the compounds tested, only Compound 31 bearing a —OCH₂— linker group (L) showed a significant delay of aggregation, and is notably more efficacious compared to Compound 1 and all other compounds bearing L=—NH—. This demonstrates the advantage of modifying linker structure to increase anti-aggregation activity. Null hypothesis testing was performed by one-way ANOVA with Dunnett's multiple comparisons. Significant difference corresponding p≤0.0001(****) is indicated.

FIG. 14: Comparison between inhibitory activities of Osimertinib derivatives designated as compound 13 and compound 9 toward the aggregation of tau in HEK biosensor cells. The number of cells with aggregates (black circles) was quantified relative to the negative control represented by cells treated with the vehicle (DMSO). The total amount of cells (grey triangles) remained close to 100% up until a compound concentration of about 4.0 μM, indicating low toxicity within the concentration range 0-4.0 μM. The half-maximal inhibitory concentration (IC₅₀) was determined by curve fitting.

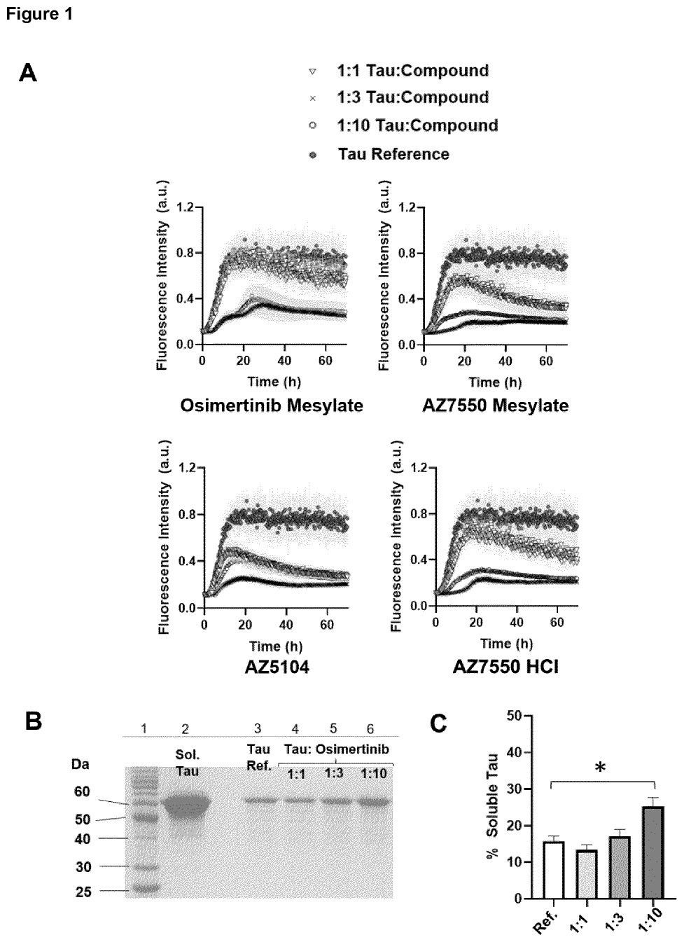

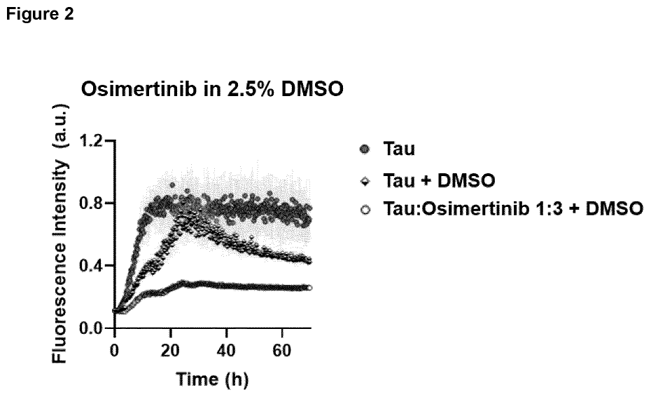

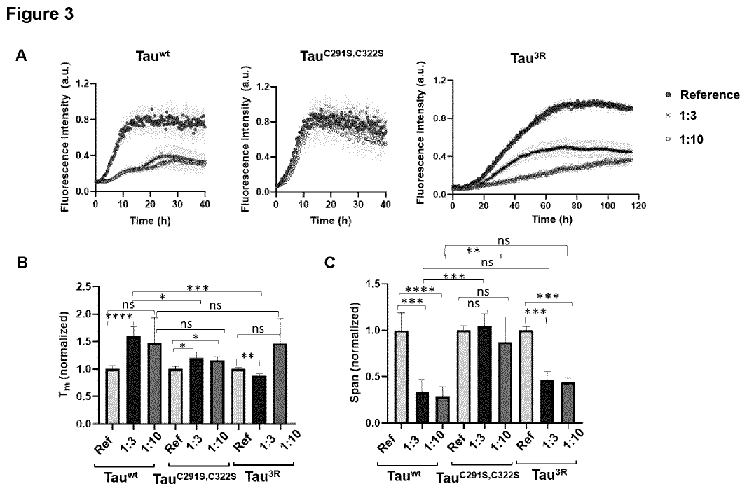

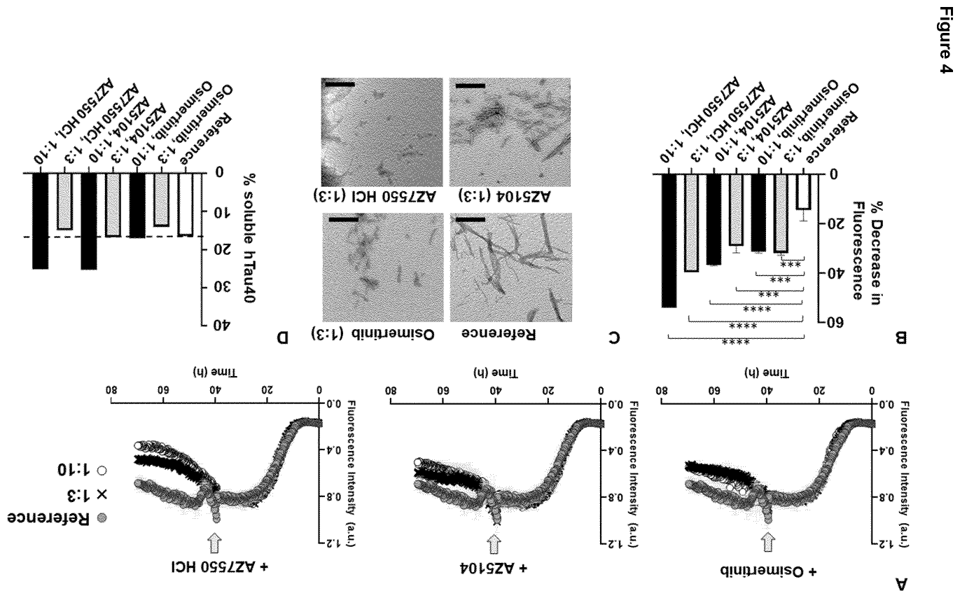

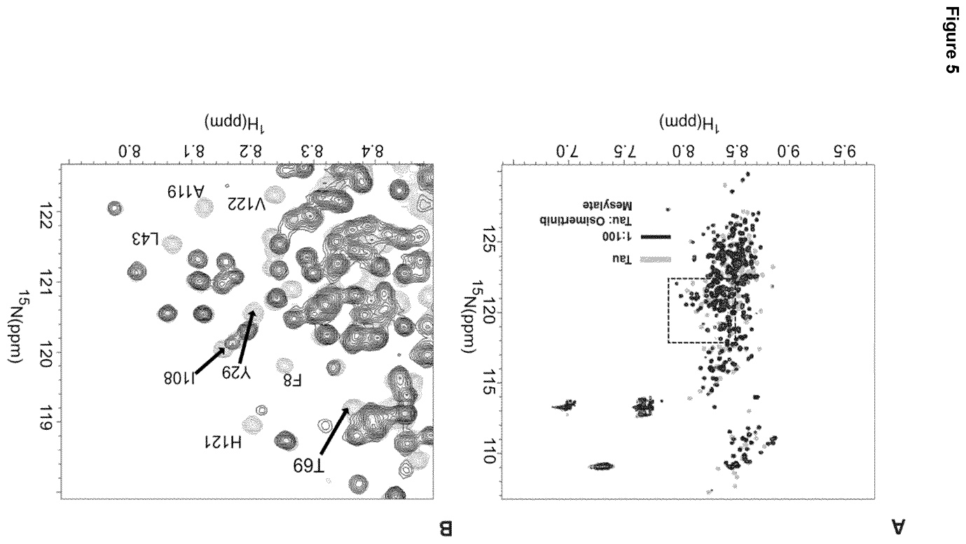

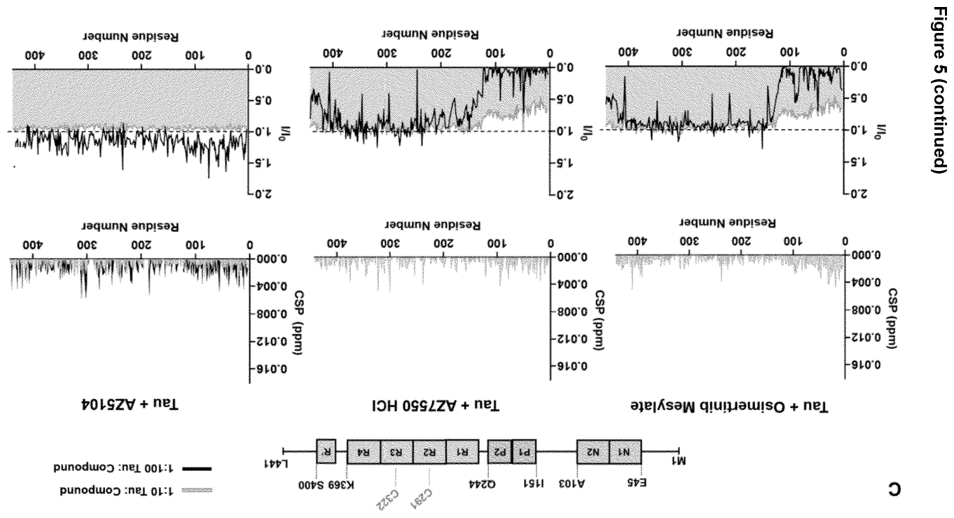

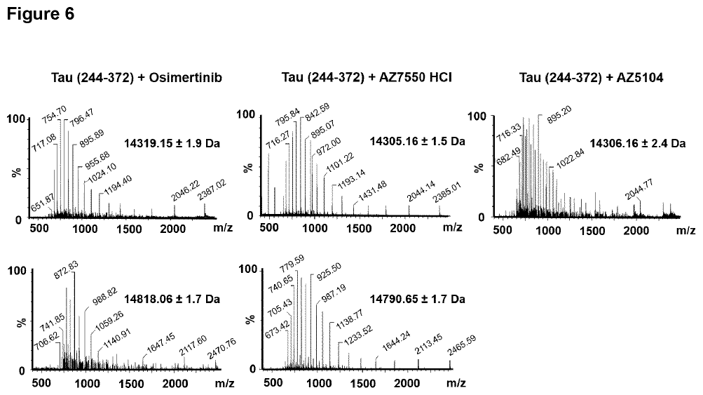

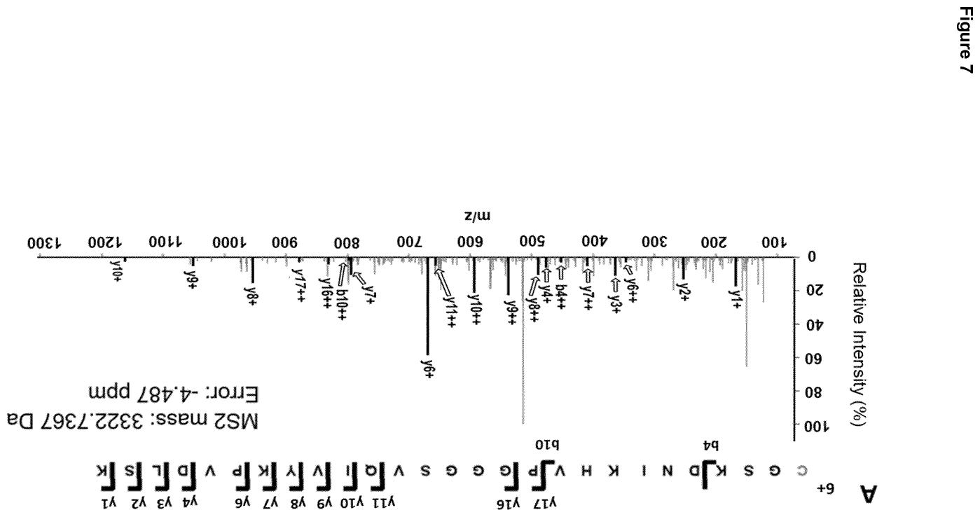

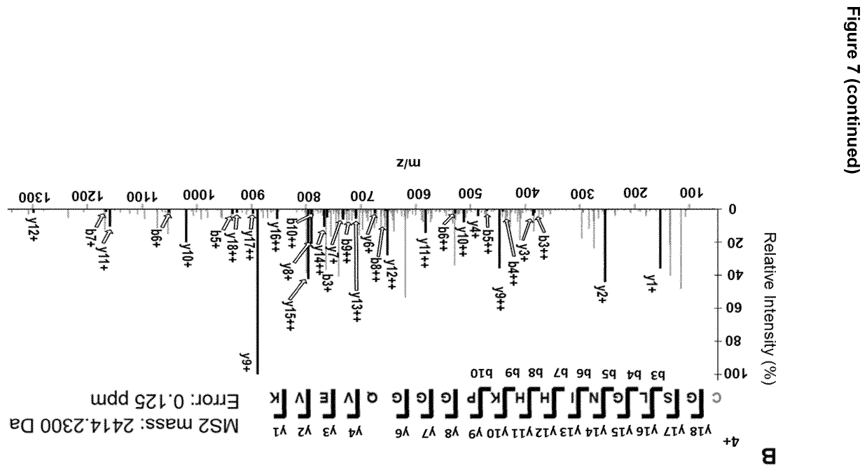

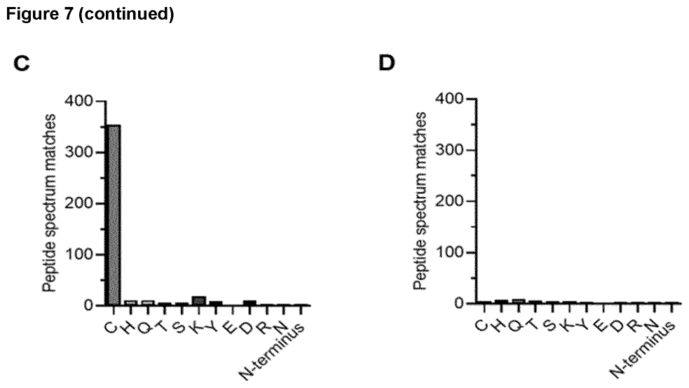

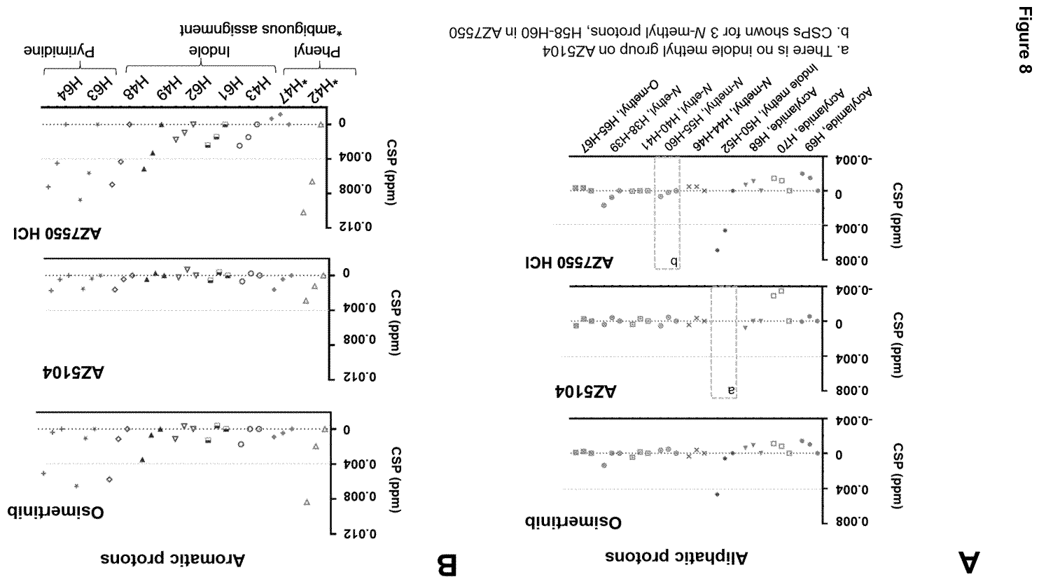

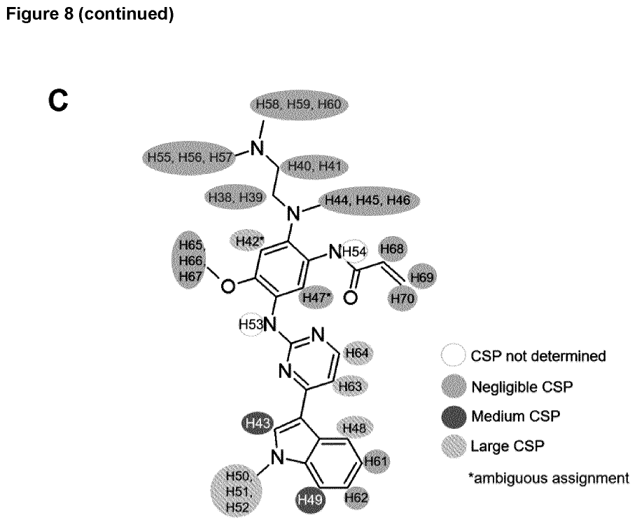

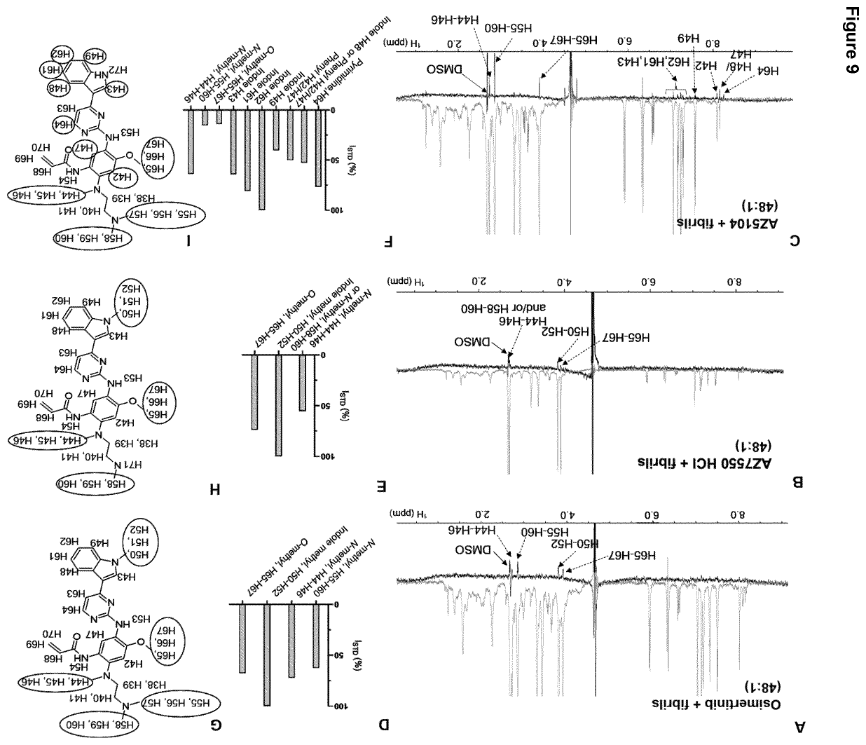

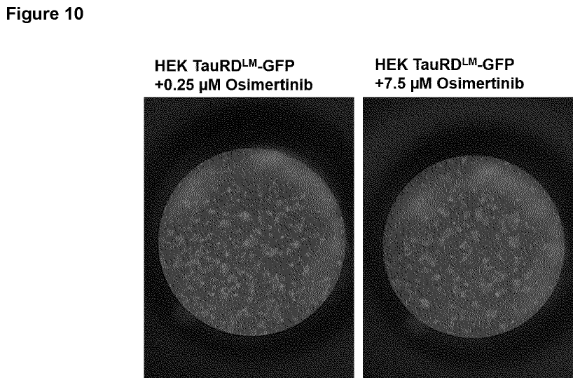

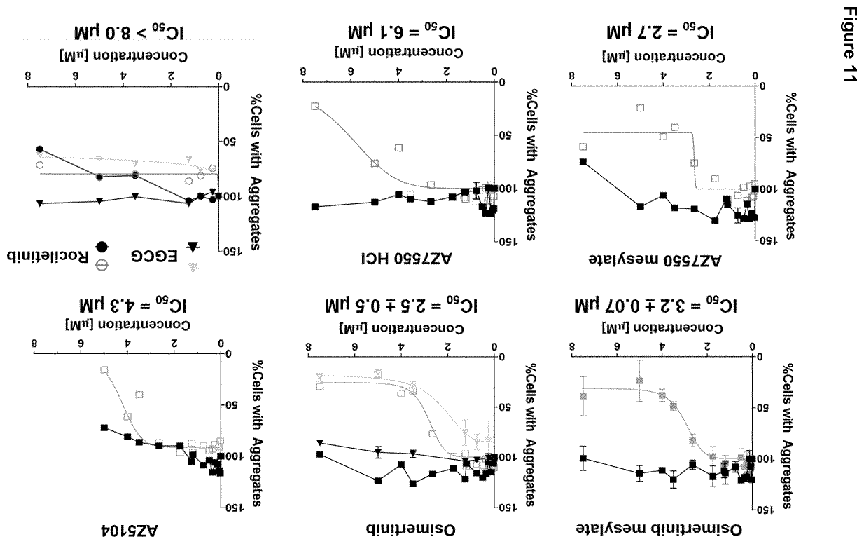

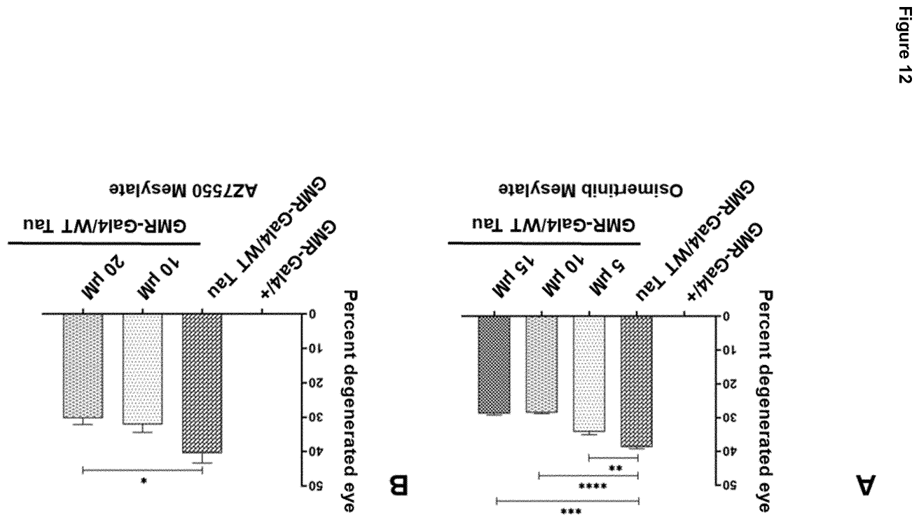

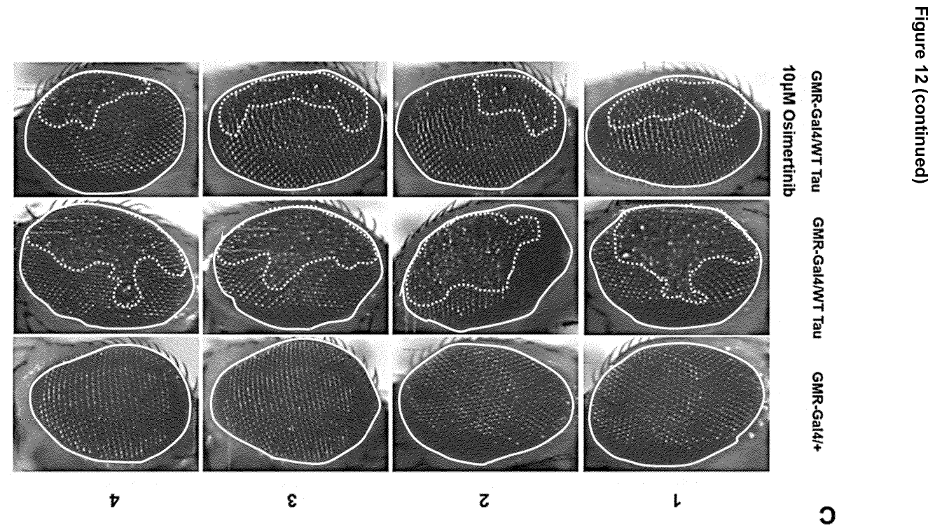

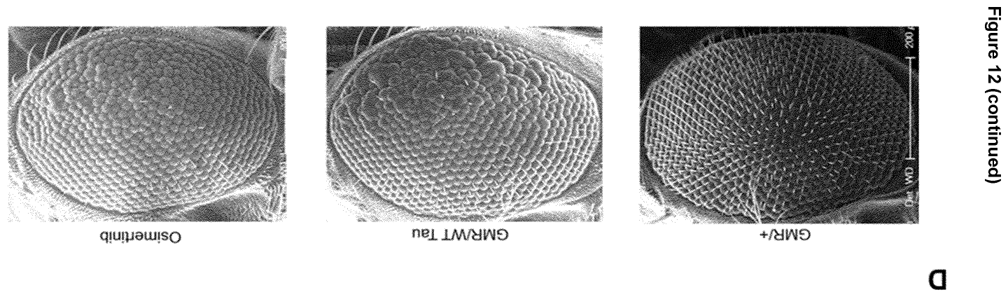

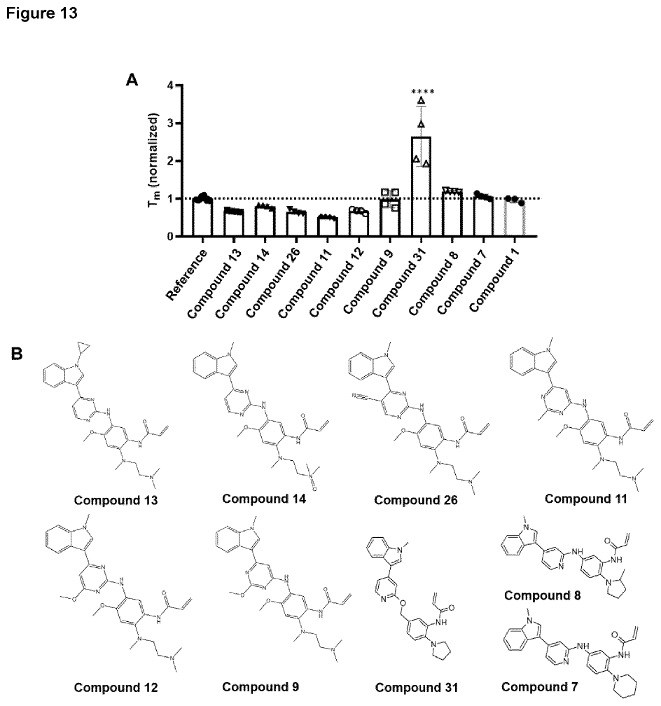

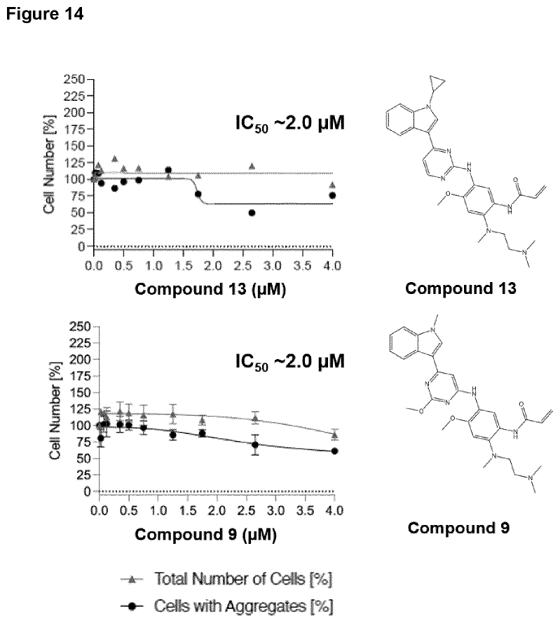

### DETAILED DESCRIPTION OF THE INVENTION

### Definitions

The term “alkyl” refers to a monoradical of a saturated straight or branched hydrocarbon. Preferably, the alkyl group comprises from 1 to 6 carbon atoms, i.e., 1, 2, 3, 4, 5, 6 carbon atoms, more preferably 1 to 4 carbon atoms, most preferably 1 carbon atom. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like.

“Pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.□

### Compounds

The compounds of the present invention are defined by formula (I) as follows:

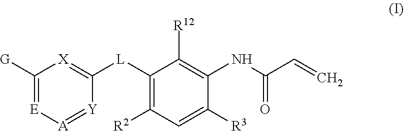

wherein
G is

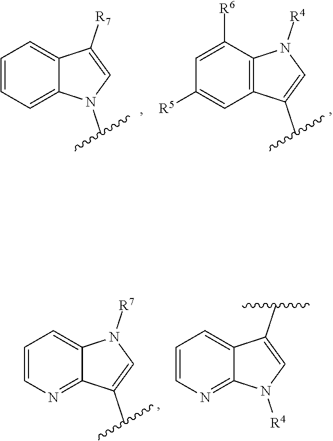

preferably

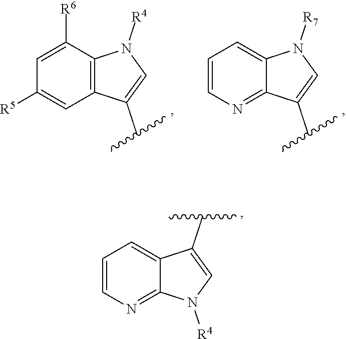

more preferably

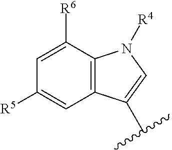

X is N or C—H, preferably N;
Y is N or C—H, preferably N;
L is

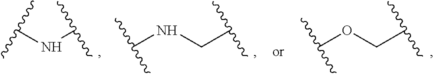

preferably

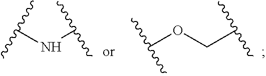

A is N or C—R; preferably C—R, more preferably CH; E is N or C—R¹; preferably C—R¹, more preferably CH;
Preferably, only one or two of A, E, X, and Y are N; A and E are not both N, and A and Y are not both N;
R is hydrogen, fluoro, chloro, bromo, —(C₁-C₆)alkyl, cyano, or —O(C₁-C₆)alkyl, preferably hydrogen;
R¹ is hydrogen, fluoro, chloro, bromo, —(C₁-C₆)alkyl, cyano, or —O(C₁-C₆)alkyl, preferably hydrogen;
R² is hydrogen, fluoro, chloro, bromo, —(C₁-C₆)alkyl, cyano, —O(C₁-C₆)alkyl, or difluoromethoxy, preferably —O(C₁-C₆)alkyl;
R³ is

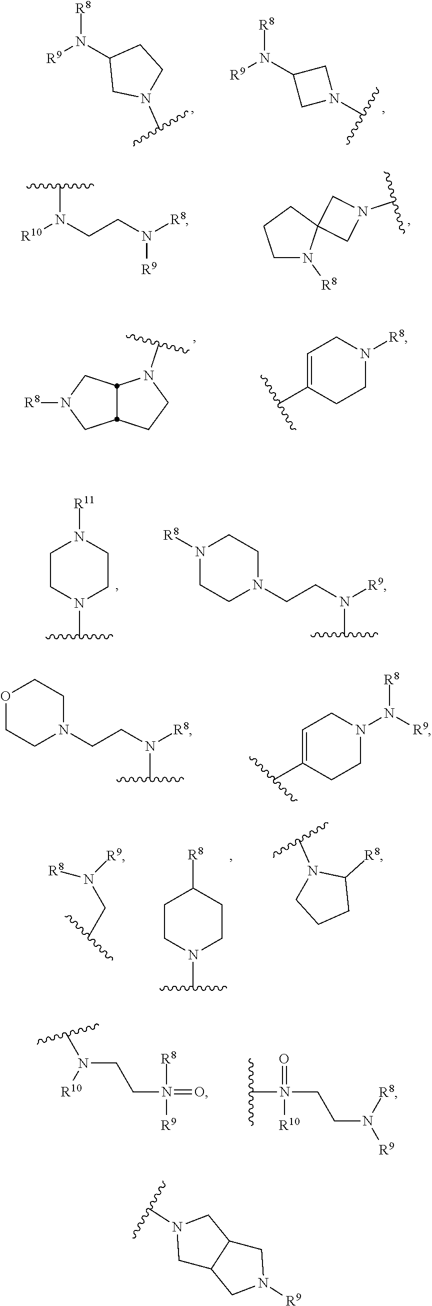

or —NR⁸R⁹, preferably

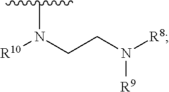

R⁴ is hydrogen, —(C₁-C₆)alkyl, or —(C₃-C₆)cycloalkyl;
R⁵ is hydrogen, fluoro, or chloro;
R⁶ is hydrogen, fluoro or chloro;
R⁷ is hydrogen, or —(C₁-C₆)alkyl;
R⁸ is hydrogen, or —(C₁-C₆)alkyl;
R⁹ is hydrogen, or —(C₁-C₆)alkyl;
R¹⁰ is hydrogen, or —(C₁-C₆)alkyl;
R¹¹ is hydrogen, —(C₁-C₆)alkyl, or —CH₂CON((C₁-C₆)alkyl)₂;
R¹² is hydrogen, or —(C₁-C₆)alkyl;
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope;
one or more fluoro atoms are replaced by the corresponding ¹⁸F isotope;
or a pharmaceutically acceptable salt thereof.

In a further embodiment at least one of the following conditions apply:
i) R is fluoro, chloro, cyano;
ii) R¹ is fluoro, methoxy;
iii) R² is hydrogen, fluoro, chloro, cyano, or methyl;
iv) R³ is pyrrolidinyl, piperidinyl, 4-methylpiperidinyl, or 2-methylpyrrolidinyl;
v) L is —OCH₂—

In a further embodiment, the compound is not selected from the group consisting of

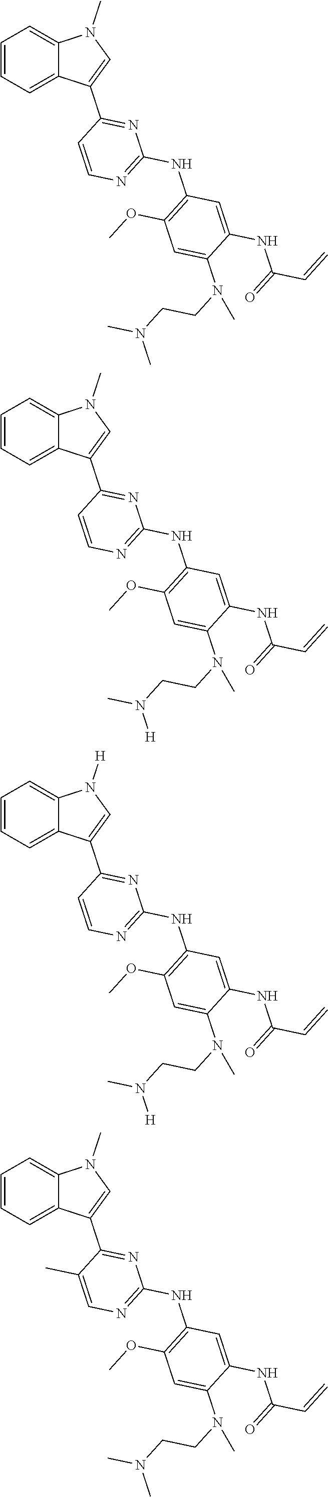

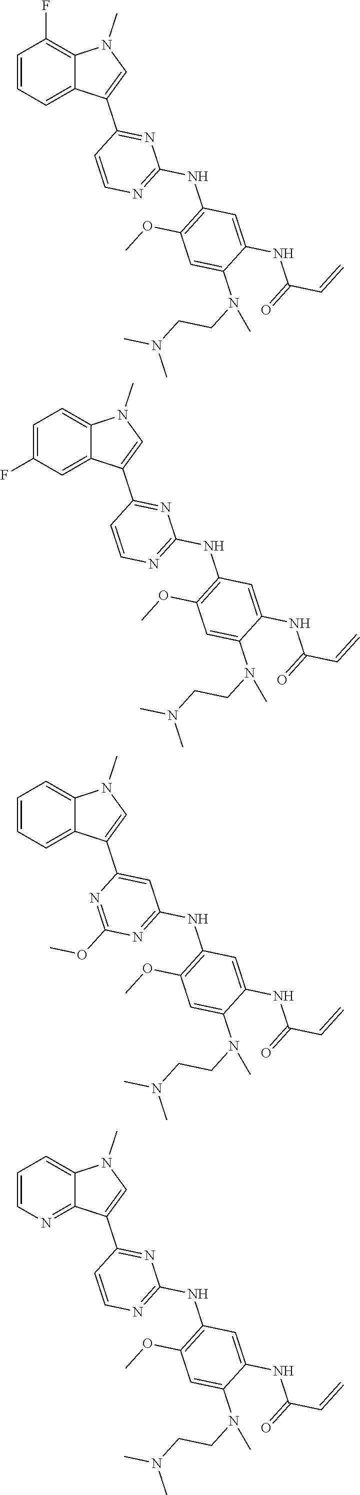

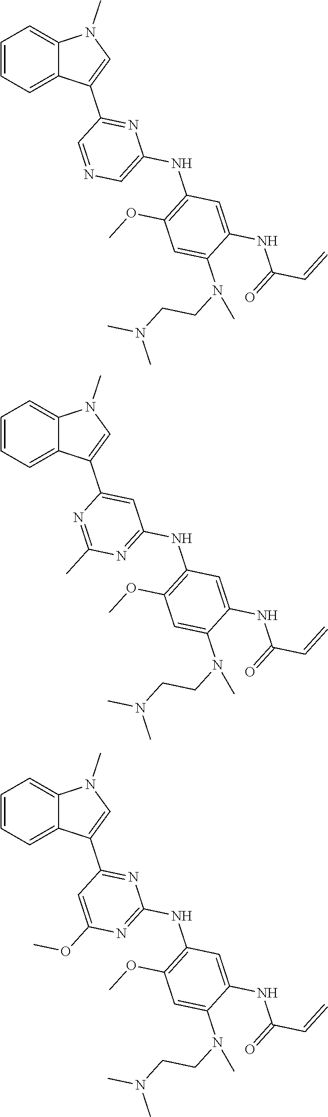

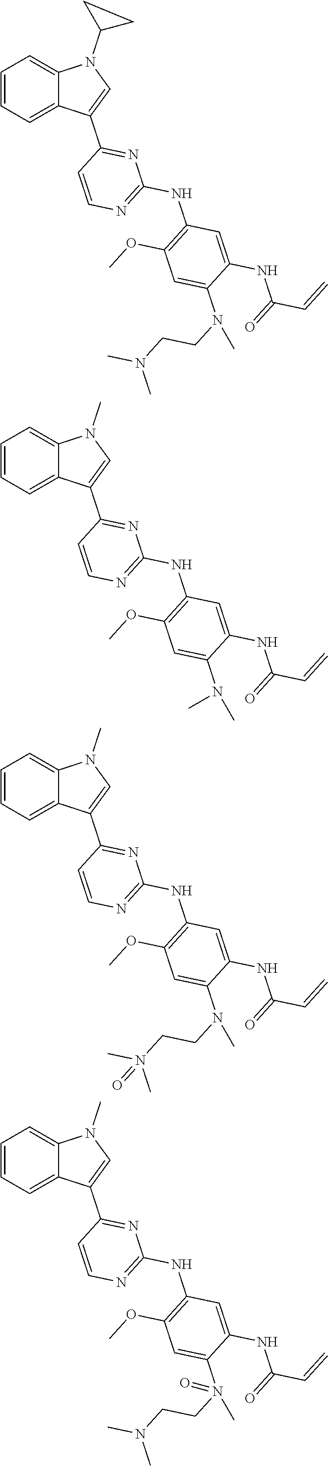

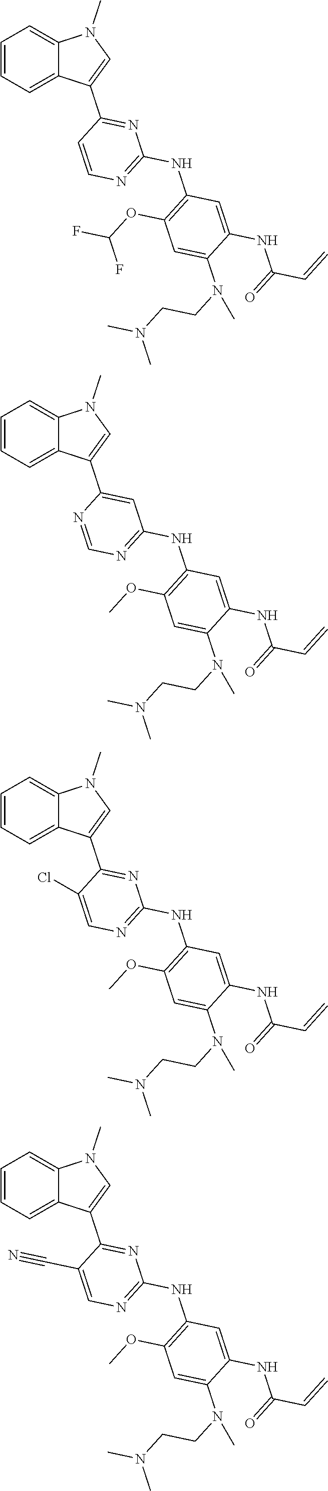 [SMILES: C=CC(=O)Nc1cc(Nc2nccc(-c3cn(C)c4ccccc34)n2)c(OC(F)F)cc1N(C)CCN(C)C ; C=CC(=O)Nc1cc(Nc2cc(-c3cn(C)c4ccccc34)ncn2)c(OC)cc1N(C)CCN(C)C ; C=CC(=O)Nc1cc(Nc2ncc(Cl)c(-c3cn(C)c4ccccc34)n2)c(OC)cc1N(C)CCN(C)C ; C=CC(=O)Nc1cc(Nc2ncc(C#N)c(-c3cn(C)c4ccccc34)n2)c(OC)cc1N(C)CCN(C)C]

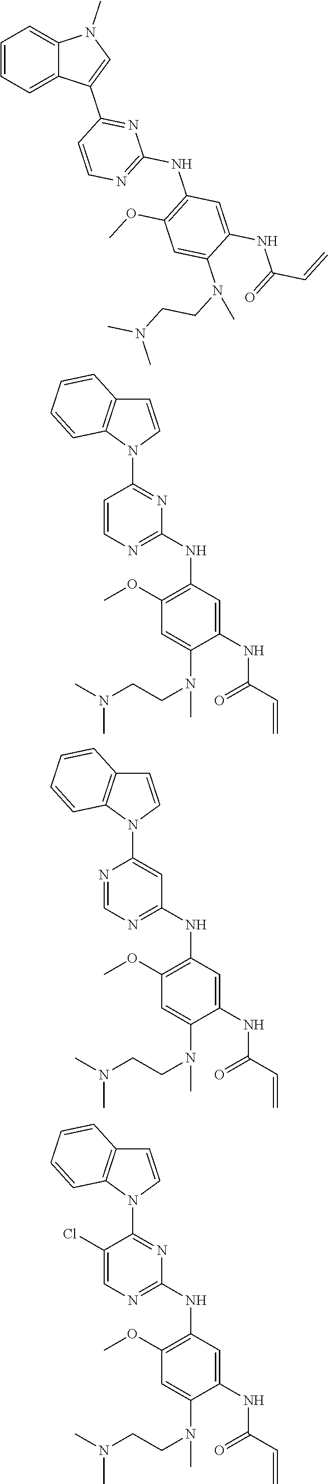

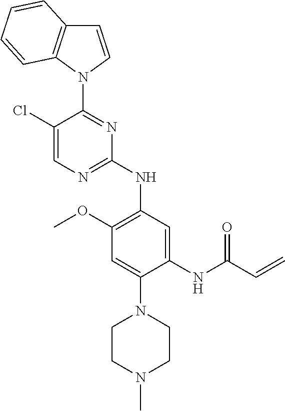 [SMILES: C=CC(=O)Nc1cc(Nc2ncc(Cl)c(-n3ccc4ccccc43)n2)c(OC)cc1N1CCN(C)CC1]

In a further embodiment
G is

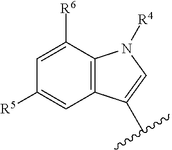

X is selected from N;
Y is selected from N;
L is

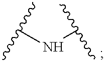 [SMILES: *N*]

A is selected from N or C—R;
E is selected from N or C—R¹
R is hydrogen;
R¹ is hydrogen;
R² is —O(C₁-C₆)alkyl;
R³ is

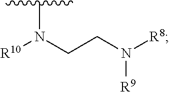

R⁴ is hydrogen, or —(C₁-C₆)alkyl;
R⁵ is hydrogen;
R⁶ is hydrogen;
R⁸ is hydrogen, or —(C₁-C₆)alkyl, preferably —(C₁-C₆)alkyl;
R⁹ is hydrogen, or —(C₁-C₆)alkyl, preferably —(C₁-C₆)alkyl;
R¹⁰ is hydrogen, or —(C₁-C₆)alkyl;

In a further embodiment,
G is

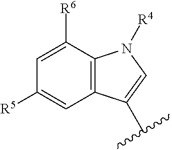

X is selected from N;
Y is selected from N;
L is

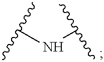 [SMILES: *N*]

A is selected from N or C—R;
E is selected from N or C—R¹
R is hydrogen;
R¹ is hydrogen;
R² is —OMe;
R³ is

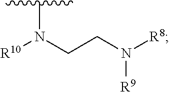

R⁴ is methyl;
R⁵ is hydrogen;
R⁶ is hydrogen;
R³ is hydrogen, or methyl;
R⁹ is methyl;
R¹⁰ is methyl.

In further embodiment, the compound is according to formula (Ia or Ib)

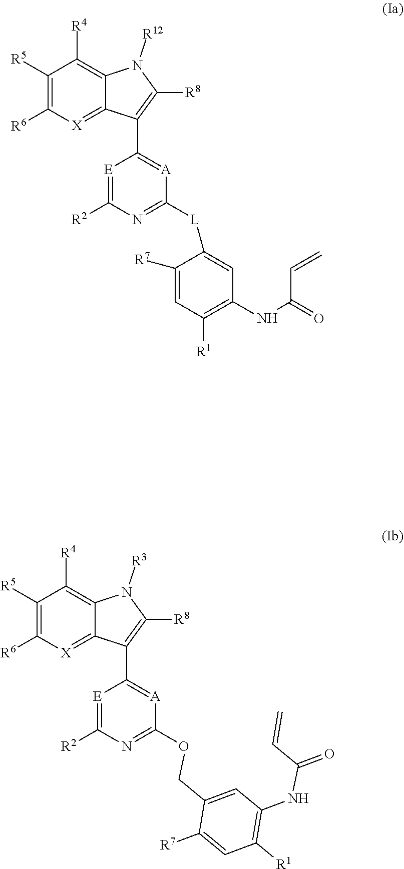

wherein
A is N or C—R; preferably C—R, more preferably CH;
E is N or C—R; preferably C—R, more preferably CH;
X is N, C—H, preferably CH;
R is hydrogen, fluoro, chloro, bromo, —(C₁-C₆)alkyl, cyano, —O(C₁-C₆)alkyl, preferably hydrogen;
L is

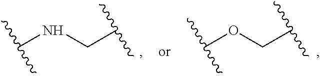

preferably

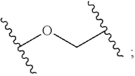 [SMILES: *CO*]

R¹ is selected from a group of alkylamines, non-aromatic heterocycles, partially or wholly deuterated alkylamines, and partially or wholly deuterated non-aromatic heterocycles including

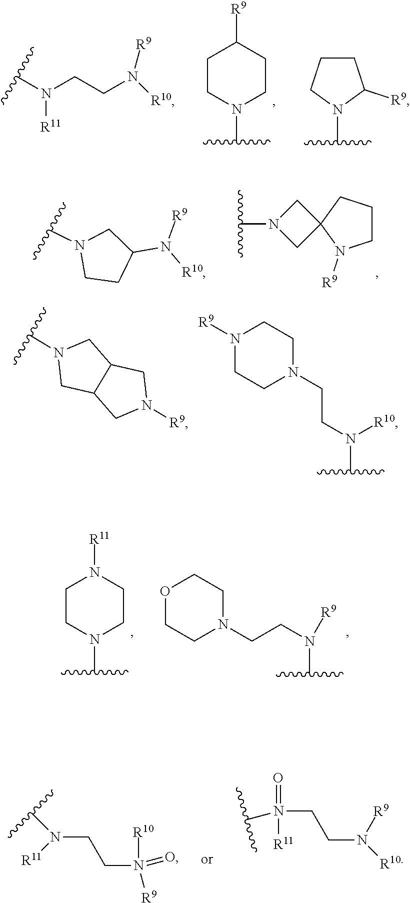

preferably

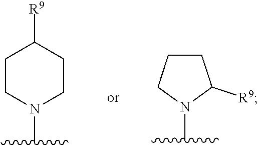

R² is hydrogen, fluoro, —(C₁-C₆)alkyl, preferably hydrogen;
R³ is hydrogen, —(C₁-C₆)alkyl, —(C₃-C₆)cycloalkyl, preferably methyl;
R⁴ is hydrogen, —(C₁-C₆)alkyl, preferably hydrogen;
R⁵ is hydrogen, —(C₁-C₆)alkyl, preferably hydrogen;
R⁶ is hydrogen, —(C₁-C₆)alkyl, preferably hydrogen;
R⁷ is hydrogen, fluoro, —O(C₁-C₆)alkyl, —(C₁-C₆)alkyl, preferably hydrogen;
R⁸ is hydrogen, —(C₁-C₆)alkyl, preferably hydrogen;
R⁹ is hydrogen, —(C₁-C₆)alkyl, preferably —(C₁-C₆)alkyl;
R¹⁰ is hydrogen, —(C₁-C₆)alkyl, preferably —(C₁-C₆)alkyl;
R¹¹ is hydrogen, —(C₁-C₆)alkyl, preferably —(C₁-C₆)alkyl;
R¹² is —(C₁-C₆)alkyl, —(C₃-C₆)cycloalkyl, preferably —(C₁-C₆)alkyl:
with the provision that A=N and

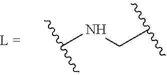 [SMILES: *CN*]

are not simultaneously present in the same compound.
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope;
one or more fluoro atoms are replaced by the corresponding ¹⁸F isotope.

Specific exemplary compounds are listed in table 1.

TABLE 1
List of compounds

| | |
|---|---|
| 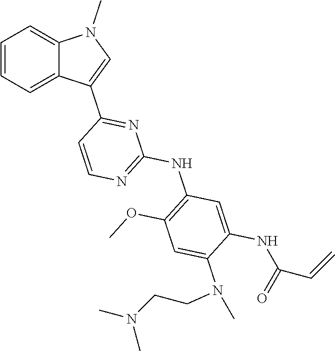 [SMILES: C=CC(=O)Nc1cc(Nc2nccc(-c3cn(C)c4ccccc34)n2)c(OC)cc1N(C)CCN(C)C] | 1 |
| 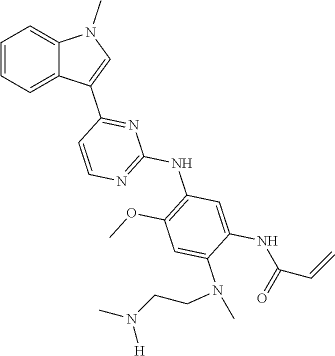 [SMILES: C=CC(=O)Nc1cc(Nc2nccc(-c3cn(C)c4ccccc34)n2)c(OC)cc1N(C)CCNC] | 2 |
| 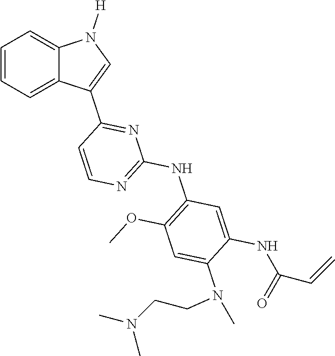 [SMILES: C=CC(=O)Nc1cc(Nc2nccc(-c3c[nH]c4ccccc34)n2)c(OC)cc1N(C)CCN(C)C] | 3 |
| 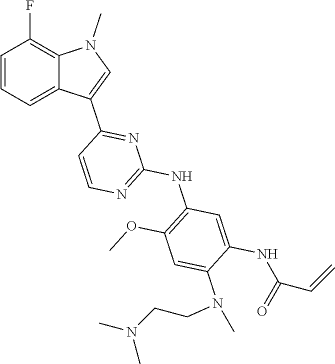 [SMILES: C=CC(=O)Nc1cc(Nc2nccc(-c3cn(C)c4c(F)cccc34)n2)c(OC)cc1N(C)CCN(C)C] | 4 |
| 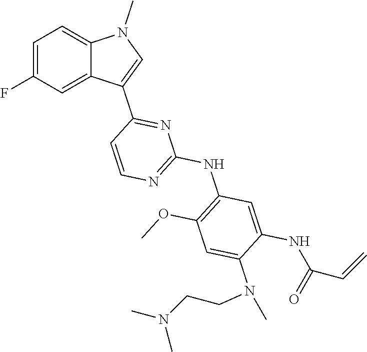 [SMILES: C=CC(=O)Nc1cc(Nc2nccc(-c3cn(C)c4ccc(F)cc34)n2)c(OC)cc1N(C)CCN(C)C] | 5 |
| 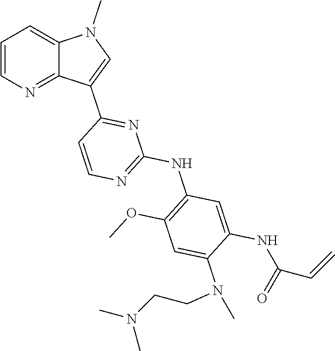 [SMILES: C=CC(=O)Nc1cc(Nc2nccc(-c3cn(C)c4cccnc34)n2)c(OC)cc1N(C)CCN(C)C] | 6 |
| 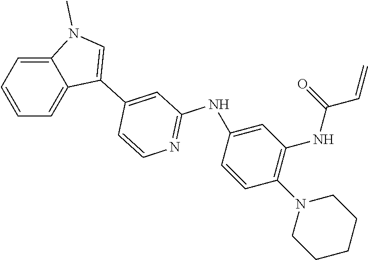 [SMILES: C=CC(=O)Nc1cc(Nc2cc(-c3cn(C)c4ccccc34)ccn2)ccc1N1CCCCC1] | 7 |
| 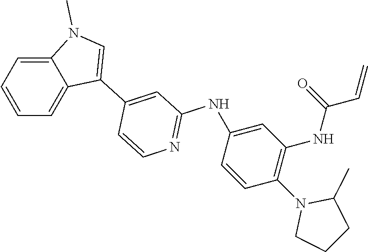 [SMILES: C=CC(=O)Nc1cc(Nc2cc(-c3cn(C)c4ccccc34)ccn2)ccc1N1CCCC1C] | 8 |
| 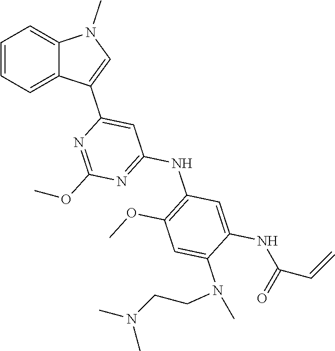 [SMILES: C=CC(=O)Nc1cc(Nc2cc(-c3cn(C)c4ccccc34)nc(OC)n2)c(OC)cc1N(C)CCN(C)C] | 9 |
| 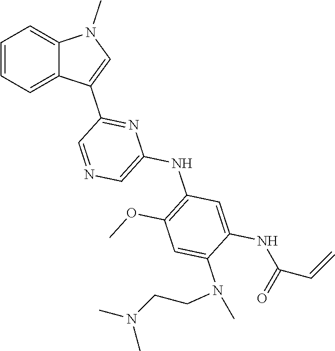 [SMILES: C=CC(=O)Nc1cc(Nc2cncc(-c3cn(C)c4ccccc34)n2)c(OC)cc1N(C)CCN(C)C] | 10 |
| 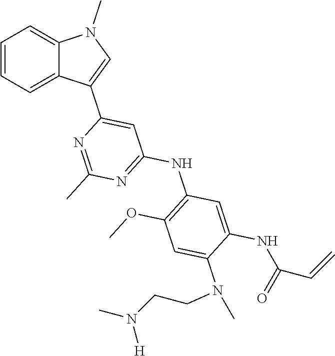 [SMILES: C=CC(=O)Nc1cc(Nc2cc(-c3cn(C)c4ccccc34)nc(C)n2)c(OC)cc1N(C)CCNC] | 11 |
| 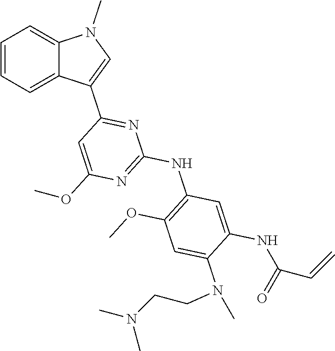 [SMILES: C=CC(=O)Nc1cc(Nc2nc(OC)cc(-c3cn(C)c4ccccc34)n2)c(OC)cc1N(C)CCN(C)C] | 12 |
| 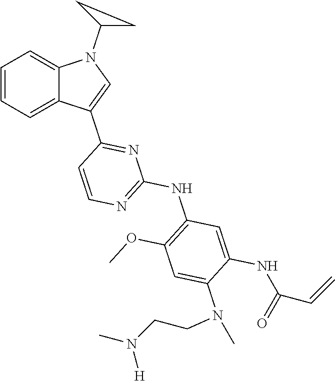 [SMILES: C=CC(=O)Nc1cc(Nc2nccc(-c3cn(C4CC4)c4ccccc34)n2)c(OC)cc1N(C)CCNC] | 13 |
| 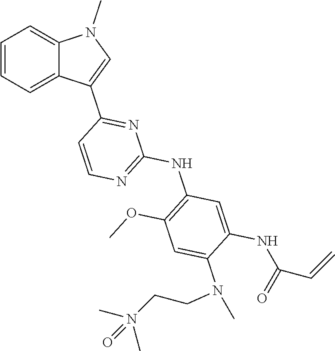 [SMILES: C=CC(=O)Nc1cc(Nc2nccc(-c3cn(C)c4ccccc34)n2)c(OC)cc1N(C)CC[N+](C)(C)[O-]] | 14 |
| 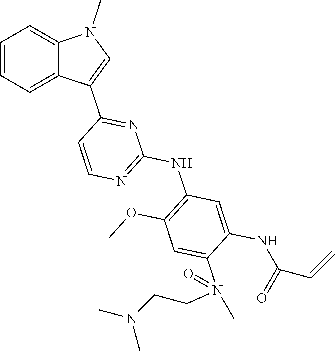 [SMILES: C=CC(=O)Nc1cc(Nc2nccc(-c3cn(C)c4ccccc34)n2)c(OC)cc1[N+](C)([O-])CCN(C)C] | 15 |
| 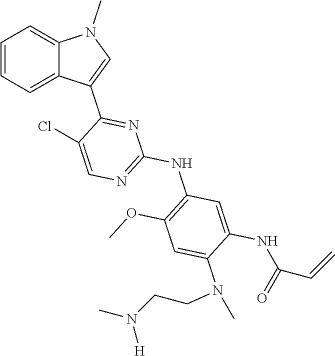 [SMILES: C=CC(=O)Nc1cc(Nc2ncc(Cl)c(-c3cn(C)c4ccccc34)n2)c(OC)cc1N(C)CCNC] | 16 |
| 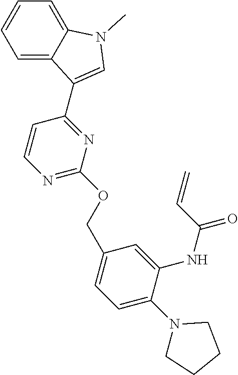 [SMILES: C=CC(=O)Nc1cc(COc2nccc(-c3cn(C)c4ccccc34)n2)ccc1N1CCCC1] | 17 |
| 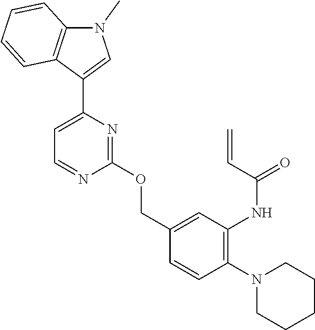 [SMILES: C=CC(=O)Nc1cc(COc2nccc(-c3cn(C)c4ccccc34)n2)ccc1N1CCCCC1] | 18 |
| 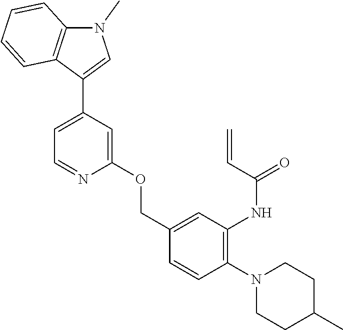 [SMILES: C=CC(=O)Nc1cc(COc2cc(-c3cn(C)c4ccccc34)ccn2)ccc1N1CCC(C)CC1] | 19 |
| 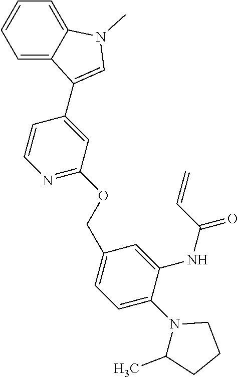 [SMILES: C=CC(=O)Nc1cc(COc2cc(-c3cn(C)c4ccccc34)ccn2)ccc1N1CCCC1C] | 20 |
| 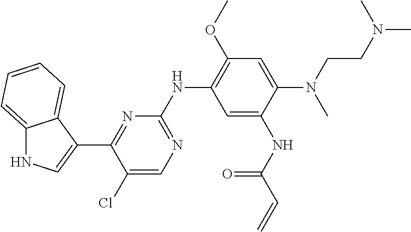 [SMILES: C=CC(=O)Nc1cc(Nc2ncc(Cl)c(-c3c[nH]c4ccccc34)n2)c(OC)cc1N(C)CCN(C)C] | 21 |
| 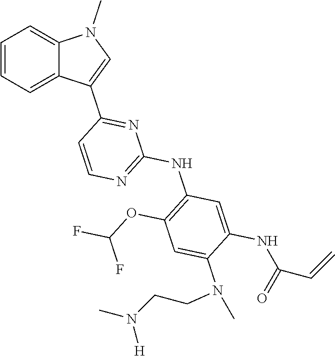 [SMILES: C=CC(=O)Nc1cc(Nc2nccc(-c3cn(C)c4ccccc34)n2)c(OC(F)F)cc1N(C)CCNC] | 22 |
| 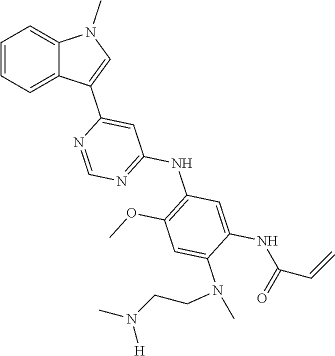 [SMILES: C=CC(=O)Nc1cc(Nc2cc(-c3cn(C)c4ccccc34)ncn2)c(OC)cc1N(C)CCNC] | 23 |
| 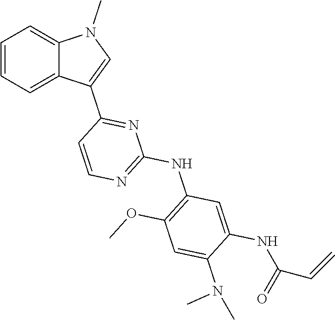 [SMILES: C=CC(=O)Nc1cc(Nc2nccc(-c3cn(C)c4ccccc34)n2)c(OC)cc1N(C)C] | 24 |
| 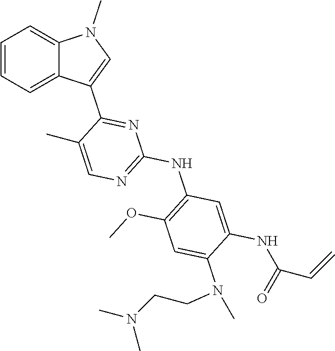 [SMILES: C=CC(=O)Nc1cc(Nc2ncc(C)c(-c3cn(C)c4ccccc34)n2)c(OC)cc1N(C)CCN(C)C] | 25 |
| 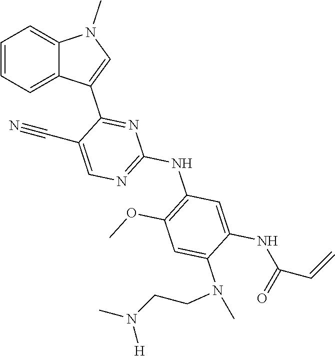 [SMILES: C=CC(=O)Nc1cc(Nc2ncc(C#N)c(-c3cn(C)c4ccccc34)n2)c(OC)cc1N(C)CCNC] | 26 |
| 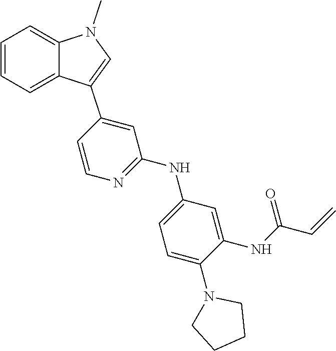 [SMILES: C=CC(=O)Nc1cc(Nc2cc(-c3cn(C)c4ccccc34)ccn2)ccc1N1CCCC1] | 27 |
| 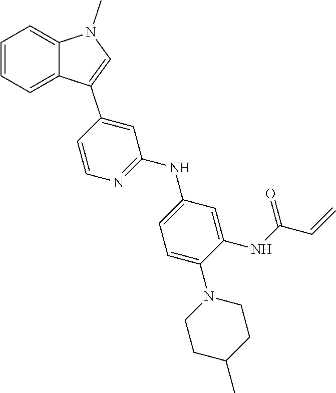 [SMILES: C=CC(=O)Nc1cc(Nc2cc(-c3cn(C)c4ccccc34)ccn2)ccc1N1CCC(C)CC1] | 28 |
| 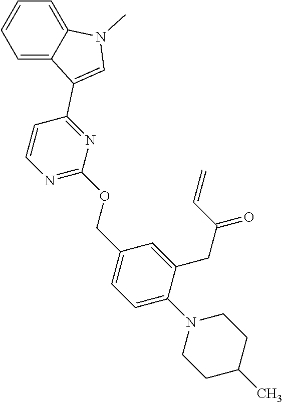 [SMILES: C=CC(=O)Cc1cc(COc2nccc(-c3cn(C)c4ccccc34)n2)ccc1N1CCC(C)CC1] | 29 |
| 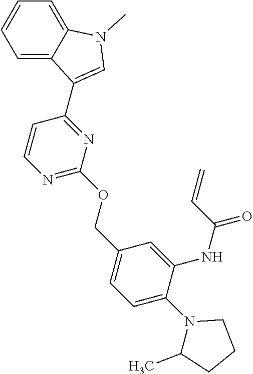 [SMILES: C=CC(=O)Nc1cc(COc2nccc(-c3cn(C)c4ccccc34)n2)ccc1N1CCCC1C] | 30 |
| 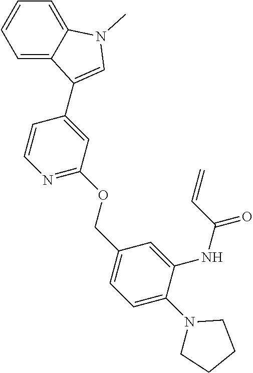 [SMILES: C=CC(=O)Nc1cc(COc2cc(-c3cn(C)c4ccccc34)ccn2)ccc1N1CCCC1] | 31 |
| 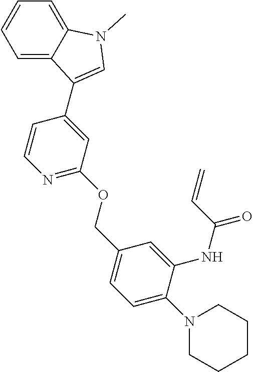 [SMILES: C=CC(=O)Nc1cc(COc2cc(-c3cn(C)c4ccccc34)ccn2)ccc1N1CCCCC1] | 32 |
| 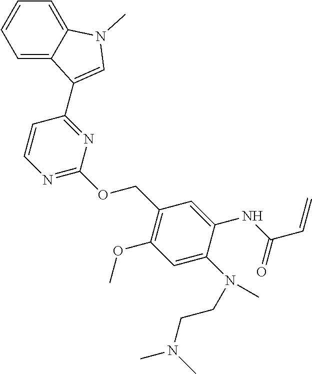 [SMILES: C=CC(=O)Nc1cc(COc2nccc(-c3cn(C)c4ccccc34)n2)c(OC)cc1N(C)CCN(C)C] | 33 |
| 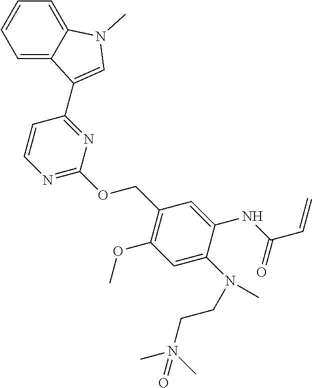 [SMILES: C=CC(=O)Nc1cc(COc2nccc(-c3cn(C)c4ccccc34)n2)c(OC)cc1N(C)CC[N+](C)(C)[O-]] | 34 |
| 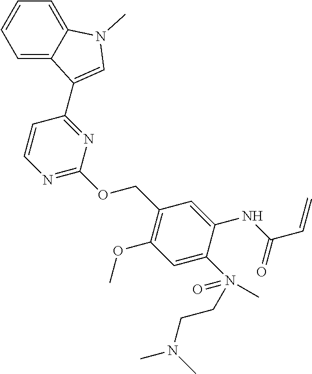 [SMILES: C=CC(=O)Nc1cc(COc2nccc(-c3cn(C)c4ccccc34)n2)c(OC)cc1[N+](C)([O-])CCN(C)C] | 35 |
| 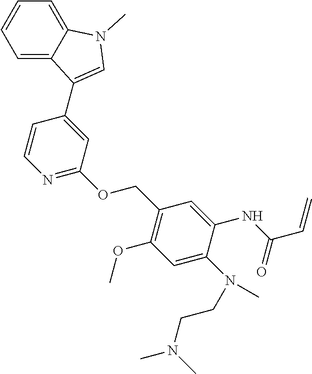 [SMILES: C=CC(=O)Nc1cc(COc2cc(-c3cn(C)c4ccccc34)ccn2)c(OC)cc1N(C)CCN(C)C] | 36 |
| 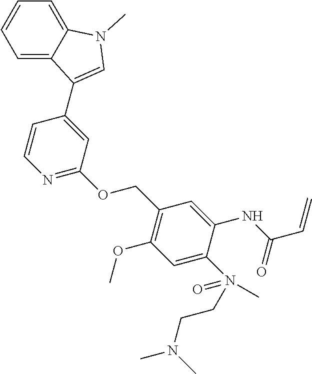 [SMILES: C=CC(=O)Nc1cc(COc2cc(-c3cn(C)c4ccccc34)ccn2)c(OC)cc1[N+](C)([O-])CCN(C)C] | 37 |
| 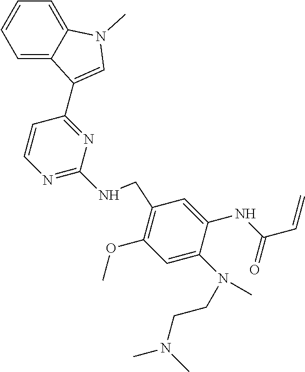 [SMILES: C=CC(=O)Nc1cc(CNc2nccc(-c3cn(C)c4ccccc34)n2)c(OC)cc1N(C)CCN(C)C] | 38 |
| 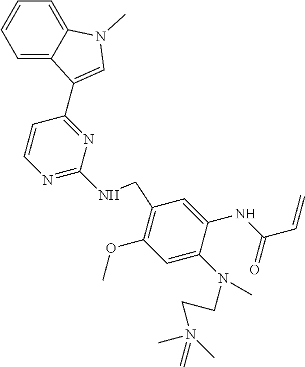 | 39 |
| 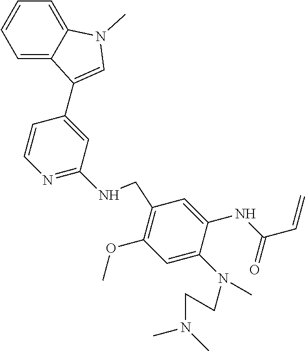 [SMILES: C=CC(=O)Nc1cc(CNc2cc(-c3cn(C)c4ccccc34)ccn2)c(OC)cc1N(C)CCN(C)C] | 40 |
| 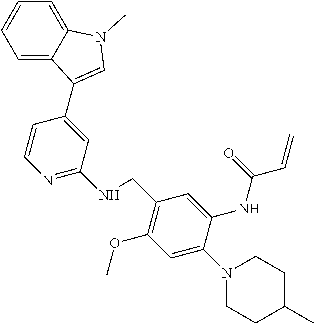 [SMILES: C=CC(=O)Nc1cc(CNc2cc(-c3cn(C)c4ccccc34)ccn2)c(OC)cc1N1CCC(C)CC1] | 41 |
| 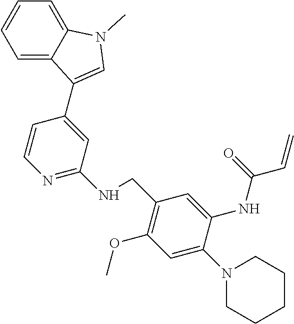 [SMILES: C=CC(=O)Nc1cc(CNc2cc(-c3cn(C)c4ccccc34)ccn2)c(OC)cc1N1CCCCC1] | 42 |
| 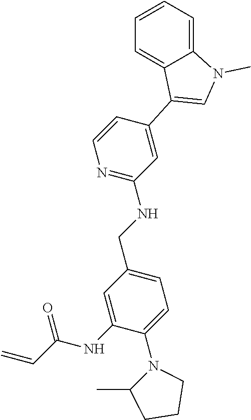 [SMILES: C=CC(=O)Nc1cc(CNc2cc(-c3cn(C)c4ccccc34)ccn2)ccc1N1CCCC1C] | 43 |
| 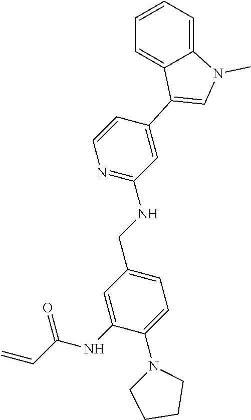 [SMILES: C=CC(=O)Nc1cc(CNc2cc(-c3cn(C)c4ccccc34)ccn2)ccc1N1CCCC1] | 44 |
| 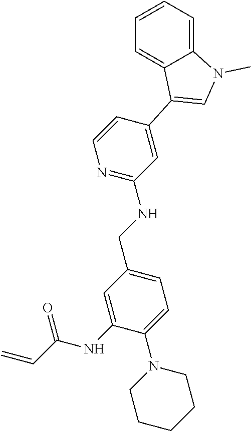 [SMILES: C=CC(=O)Nc1cc(CNc2cc(-c3cn(C)c4ccccc34)ccn2)ccc1N1CCCCC1] | 45 |
| 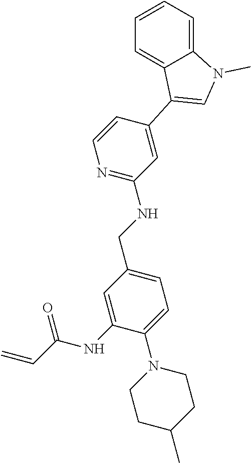 [SMILES: C=CC(=O)Nc1cc(CNc2cc(-c3cn(C)c4ccccc34)ccn2)ccc1N1CCC(C)CC1] | 46 |
| 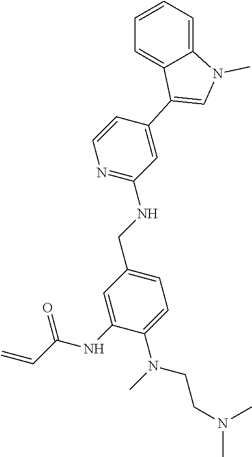 [SMILES: C=CC(=O)Nc1cc(CNc2cc(-c3cn(C)c4ccccc34)ccn2)ccc1N(C)CCN(C)C] | 47 |
| 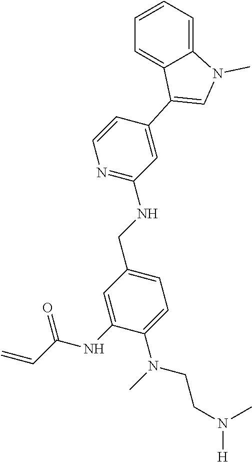 [SMILES: C=CC(=O)Nc1cc(CNc2cc(-c3cn(C)c4ccccc34)ccn2)ccc1N(C)CCNC] | 48 |
| 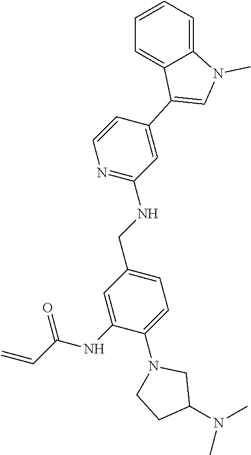 [SMILES: C=CC(=O)Nc1cc(CNc2cc(-c3cn(C)c4ccccc34)ccn2)ccc1N1CCC(N(C)C)C1] | 49 |
| 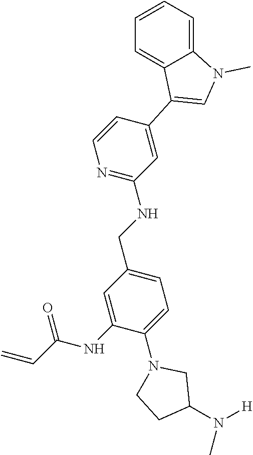 [SMILES: C=CC(=O)Nc1cc(CNc2cc(-c3cn(C)c4ccccc34)ccn2)ccc1N1CCC(NC)C1] | 50 |
| 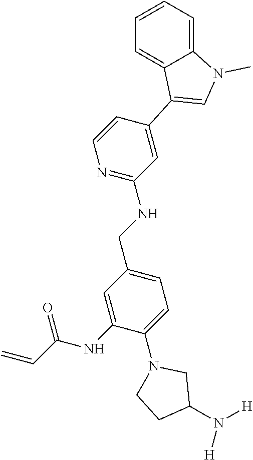 [SMILES: C=CC(=O)Nc1cc(CNc2cc(-c3cn(C)c4ccccc34)ccn2)ccc1N1CCC(N)C1] | 51 |
| 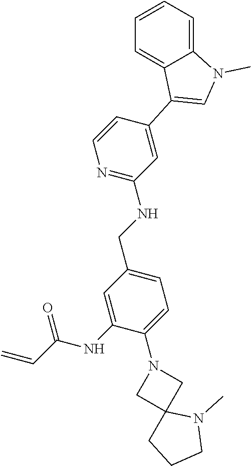 [SMILES: C=CC(=O)Nc1cc(CNc2cc(-c3cn(C)c4ccccc34)ccn2)ccc1N1CC2(CCCN2C)C1] | 52 |
| 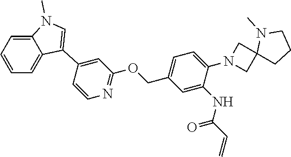 [SMILES: C=CC(=O)Nc1cc(COc2cc(-c3cn(C)c4ccccc34)ccn2)ccc1N1CC2(CCCN2C)C1] | 53 |
| 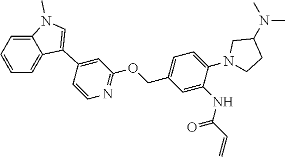 [SMILES: C=CC(=O)Nc1cc(COc2cc(-c3cn(C)c4ccccc34)ccn2)ccc1N1CCC(N(C)C)C1] | 54 |
| 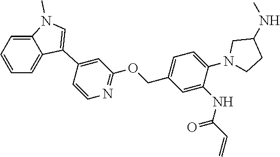 [SMILES: C=CC(=O)Nc1cc(COc2cc(-c3cn(C)c4ccccc34)ccn2)ccc1N1CCC(NC)C1] | 55 |
| 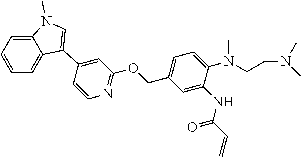 [SMILES: C=CC(=O)Nc1cc(COc2cc(-c3cn(C)c4ccccc34)ccn2)ccc1N(C)CCN(C)C] | 56 |
| 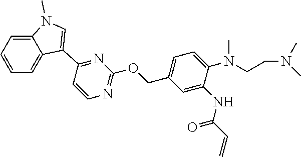 [SMILES: C=CC(=O)Nc1cc(COc2nccc(-c3cn(C)c4ccccc34)n2)ccc1N(C)CCN(C)C] | 57 |
| 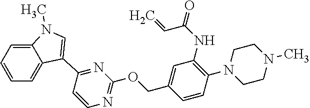 [SMILES: C=CC(=O)Nc1cc(COc2nccc(-c3cn(C)c4ccccc34)n2)ccc1N1CCN(C)CC1] | 58 |
| 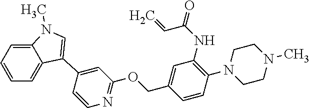 [SMILES: C=CC(=O)Nc1cc(COc2cc(-c3cn(C)c4ccccc34)ccn2)ccc1N1CCN(C)CC1] | 59 |
| 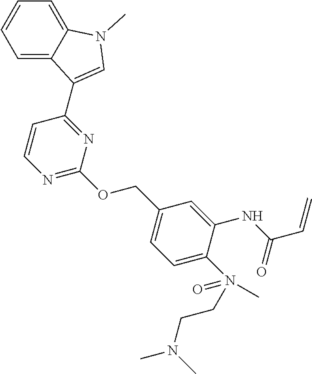 [SMILES: C=CC(=O)Nc1cc(COc2nccc(-c3cn(C)c4ccccc34)n2)ccc1[N+](C)([O-])CCN(C)C] | 60 |
| 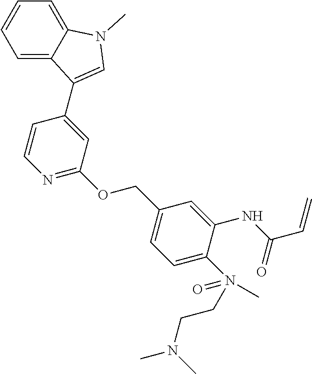 [SMILES: C=CC(=O)Nc1cc(COc2cc(-c3cn(C)c4ccccc34)ccn2)ccc1[N+](C)([O-])CCN(C)C] | 61 |
| 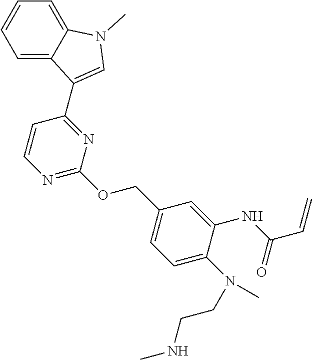 [SMILES: C=CC(=O)Nc1cc(COc2nccc(-c3cn(C)c4ccccc34)n2)ccc1N(C)CCNC] | 62 |
| 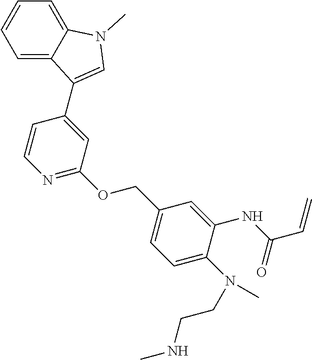 [SMILES: C=CC(=O)Nc1cc(COc2cc(-c3cn(C)c4ccccc34)ccn2)ccc1N(C)CCNC] | 63 |
| 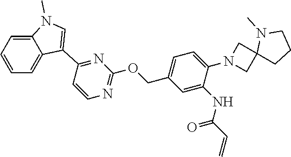 [SMILES: C=CC(=O)Nc1cc(COc2nccc(-c3cn(C)c4ccccc34)n2)ccc1N1CC2(CCCN2C)C1] | 64 |
| 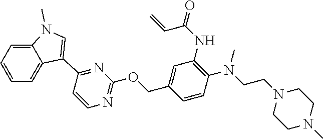 [SMILES: C=CC(=O)Nc1cc(COc2nccc(-c3cn(C)c4ccccc34)n2)ccc1N(C)CCN1CCN(C)CC1] | 65 |
| 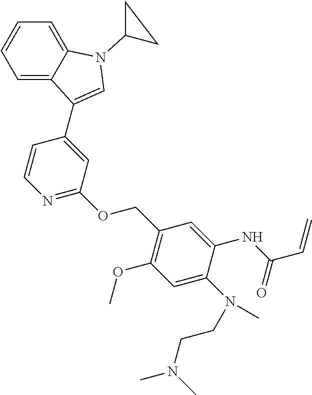 [SMILES: C=CC(=O)Nc1cc(COc2cc(-c3cn(C4CC4)c4ccccc34)ccn2)c(OC)cc1N(C)CCN(C)C] | 66 |
| 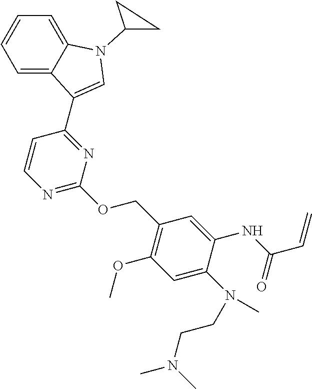 [SMILES: C=CC(=O)Nc1cc(COc2nccc(-c3cn(C4CC4)c4ccccc34)n2)c(OC)cc1N(C)CCN(C)C] | 67 |
| 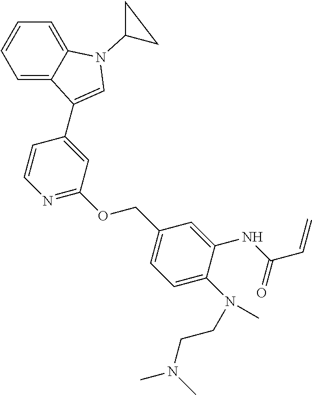 [SMILES: C=CC(=O)Nc1cc(COc2cc(-c3cn(C4CC4)c4ccccc34)ccn2)ccc1N(C)CCN(C)C] | 68 |
| 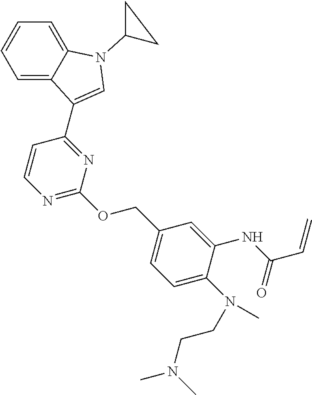 [SMILES: C=CC(=O)Nc1cc(COc2nccc(-c3cn(C4CC4)c4ccccc34)n2)ccc1N(C)CCN(C)C] | 69 |
| 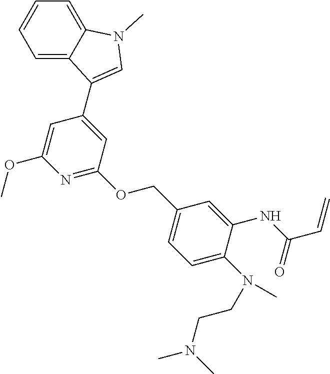 [SMILES: C=CC(=O)Nc1cc(COc2cc(-c3cn(C)c4ccccc34)cc(OC)n2)ccc1N(C)CCN(C)C] | 70 |
| 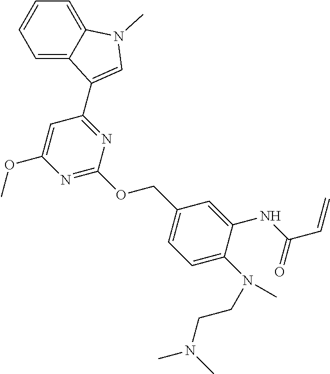 [SMILES: C=CC(=O)Nc1cc(COc2nc(OC)cc(-c3cn(C)c4ccccc34)n2)ccc1N(C)CCN(C)C] | 71 |
| 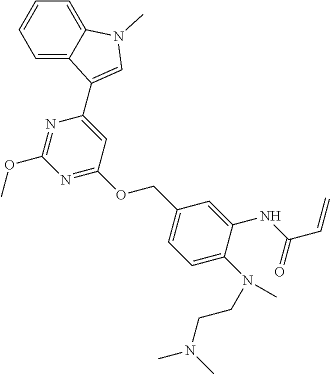 [SMILES: C=CC(=O)Nc1cc(COc2cc(-c3cn(C)c4ccccc34)nc(OC)n2)ccc1N(C)CCN(C)C] | 72 |
| 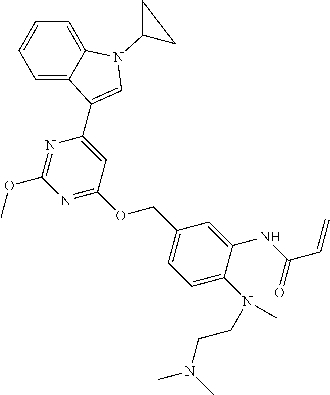 [SMILES: C=CC(=O)Nc1cc(COc2cc(-c3cn(C4CC4)c4ccccc34)nc(OC)n2)ccc1N(C)CCN(C)C] | 73 |
| 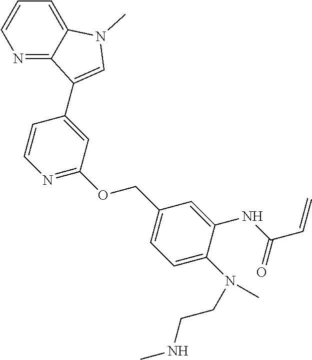 [SMILES: C=CC(=O)Nc1cc(COc2cc(-c3cn(C)c4cccnc34)ccn2)ccc1N(C)CCNC] | 74 |
| 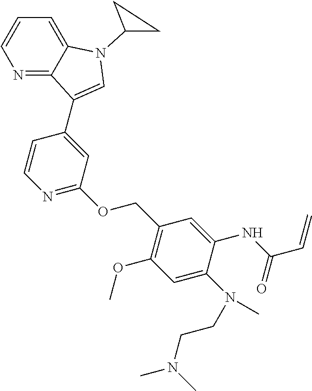 [SMILES: C=CC(=O)Nc1cc(COc2cc(-c3cn(C4CC4)c4cccnc34)ccn2)c(OC)cc1N(C)CCN(C)C] | 75 |
| 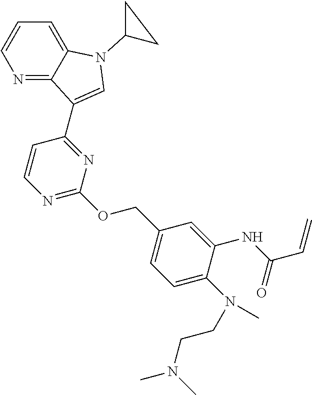 [SMILES: C=CC(=O)Nc1cc(COc2nccc(-c3cn(C4CC4)c4cccnc34)n2)ccc1N(C)CCN(C)C] | 76 |
| 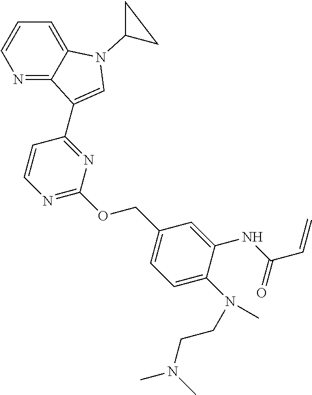 [SMILES: C=CC(=O)Nc1cc(COc2nccc(-c3cn(C4CC4)c4cccnc34)n2)ccc1N(C)CCN(C)C] | 77 |
| 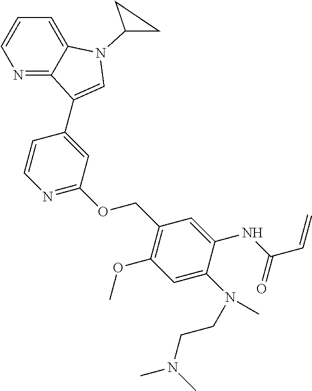 [SMILES: C=CC(=O)Nc1cc(COc2cc(-c3cn(C4CC4)c4cccnc34)ccn2)c(OC)cc1N(C)CCN(C)C] | 78 |
| 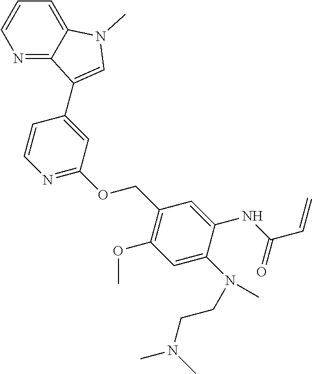 [SMILES: C=CC(=O)Nc1cc(COc2cc(-c3cn(C)c4cccnc34)ccn2)c(OC)cc1N(C)CCN(C)C] | 79 |
| 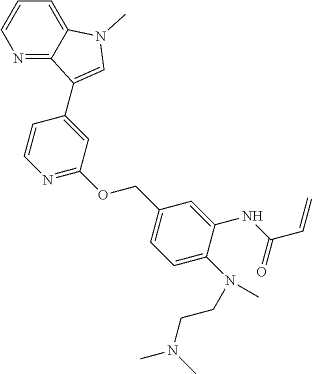 [SMILES: C=CC(=O)Nc1cc(COc2cc(-c3cn(C)c4cccnc34)ccn2)ccc1N(C)CCN(C)C] | 80 |
| 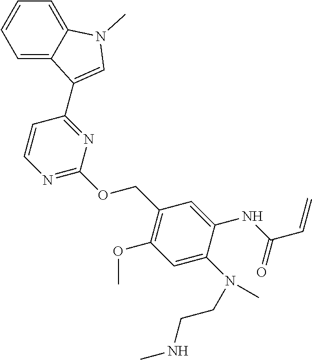 [SMILES: C=CC(=O)Nc1cc(COc2nccc(-c3cn(C)c4ccccc34)n2)c(OC)cc1N(C)CCNC] | 81 |
| 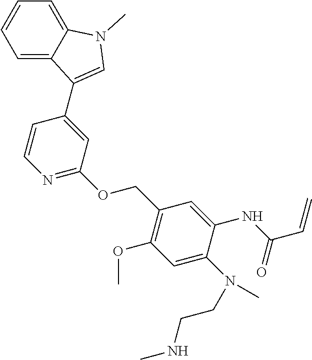 [SMILES: C=CC(=O)Nc1cc(COc2cc(-c3cn(C)c4ccccc34)ccn2)c(OC)cc1N(C)CCNC] | 82 |
| 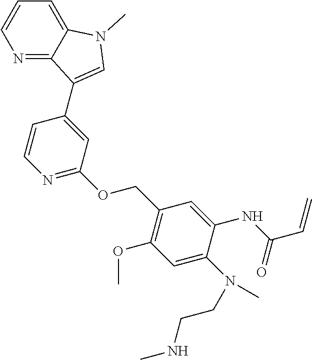 [SMILES: C=CC(=O)Nc1cc(COc2cc(-c3cn(C)c4cccnc34)ccn2)c(OC)cc1N(C)CCNC] | 83 |

### Medical Application

The compounds according to formula (I), (la) and (Ib) as described above or the pharmaceutical compositions of the present invention are for use in preventing or treating a tauopathy.

In general, a tauopathy belongs to a class of diseases associated with the pathological aggregation of tau protein in neuropil threads, pretangles, neurofibrillary or gliofibrillary tangles in the nervous system. The tau protein undergoes several post-translational modifications, including phosphorylation, acetylation, ubiquitination and myristoylation with phosphorylation being a major modification of tau and its cellular functions. In tauopathies, tau aggregates are found to be extensively phosphorylated at several serine, threonine or tyrosine residues, and acetylated and ubiquitinated at several lysine residues^{11, 12, 13, 14} Hyperphosphorylation and acetylation of tau decrease its ability to bind to microtubules thereby disturbing transport of cargos and induces the pathogenic accumulation of tau within the cell.

Thus, the term tauopathy encompasses both the loss-of-function effects on the microtubules and the gain-of-function effects of the toxic tau species. In detail, the consequences of tau hyperphosphorylation and acetylation, and potentially other post-translational modifications, include not only loss of its function to bind and stabilize microtubule but also a gain of neurotoxic properties by tau aggregation into neuropil threads, pretangles, neurofibrillary or gliofibrillary tangles.

Preferably, preventing or treating a tauopathy^{15,16} with the compounds according to formula (I) of the present invention comprises a causal treatment of the tauopathy.

Preferably, preventing or treating a tauopathy comprises inhibiting the cellular activity of tau by binding of the compound according to formula (I), (Ia) or (Ib) to tau. Binding to tau may comprise covalent binding of the compound according to general formula (I) to tau, preferably the binding comprises a thiol-Michael addition of an —SH group of tau to the acrylamide group in the compound according to formula (I).

Preferably, the tauopathy is dementia-associated tauopathy.

Preferably, the tauopathy is selected from the group consisting of Alzheimer's disease, frontotemporal dementia, primary age-related tauopathy (PART), familial British dementia (FBD), familial Danish dementia (FDD), chronic traumatic encephalopathy (CTE)¹⁵, progressive supranuclear palsy (PSP)¹⁵, corticobasal degeneration (CBD)¹⁵, Pick's disease (PiD)¹⁵, dementia with Lewy Bodies (DLB)¹⁵, progressive supranuclear palsy (PSP)¹⁵, globular glial tauopathy (GGT)¹⁵, tauopathy with hippocampal 4-repeat tau immunoreactive spherical inclusions, limbic-predominant neuronal inclusion body 4R tauopathy (LNT), frontotemporal dementia and parkinsonism linked to chromosome 17 (FTDP-17)¹⁷, argyrophilic grain disease (AGD)¹⁷, Huntington disease¹⁷, glial globular tauopathy, neuro-astroglial tauopathy variants in the elderly, familial behavioural variant frontotemporal dementia associated with astrocyte-predominant tauopathy, amyotrophic lateral sclerosis (ALS)¹⁸, spinocerebellar ataxia type 11, spinal muscular atrophy (SMA), progressive ataxia and palatal tremor-related tauopathy, cerebral amyloid angiopathy (CAA), IgLON5 antibody-related tauopathy, Chromosome 21 trisomy-related Alzheimer's disease (Down's syndrome), vascular dementia, cerebral amyloid angiopathy, Gerstmann-Straussler-Scheinker disease (GSS), Creutzfeldt-Jakob disease, fatal familial insomnia, Kuru, Niemann-Pick disease type C-related tauopathy, Nodding syndrome, Non-Guamanian motor neuron disease with neurofibrillary tangles, Parkinson's disease (PD)¹⁹, Parkinson's disease with dementia¹⁷, Parkinsonism-dementia of Guam¹⁷, Guadeloupean parkinsonism¹⁷, Kosaka-Shibayama disease¹⁷, post-encephalitic parkinsonism¹⁷, SYNJ1 (PARK20) early-onset recessive form of parkinsonism with seizures and dystonia associated nigral tau pathology, multiple system atrophy, tau pathology associated with familial parkinsonism and progressive respiratory failure, limbic predominant neuronal-glial tau pathology in TARDBP gene mutations (1383; P112H), neuronal 4R tau pathology in fatal familial insomnia (PRNP D178N mutation), tau pathology associated with ADCY5 dyskinesia, tau pathology in chronic temporal lobe epilepsy, neurodegeneration with brain iron accumulation (NBIA), tau pathology in NBIA PANK2 and WDR45 gene mutations, tau pathology in NBIA PLA2G6 mutation, tau pathology in NBIA associated with autosomal-dominant mitochondrial membrane protein-associated neurodegeneration (MPAN), neuropil threads pretangles and neurofibrillary tangles in human immunodeficiency virus (HIV)-negative opiate abusers²⁰, tau pathology associated with acquired immunodeficiency syndrome (AIDS)²⁰, diffuse neurofibrillary tangles with calcification, progressive ataxia and palatal tremor, SLC9A6-related parkinsonism, tau pathology associated with SPG7 gene mutation, striatal 4R tau pathology associated to X-linked parkinsonism with spasticity (ATP6AP2), tau pathology associated with SPAST gene-related hereditary spastic paraplegia, autism, autism spectrum disorders, retinal tauopathy²¹, West Nile encephalomyelitis, TTBK2 gene-related spinocerebellar ataxia 11, herpes simplex encephalitis, tangle-only dementia (TOD), aging-related tau astrogliopathy (ARTAG)¹⁷, hippocampal tauopathy, subacute sclerosing panencephalitis (SSPE)-related tauopathy, FTLD-C9ORF72, Christianson syndrome, vacuolar tauopathy, lytico-bodig disease, ganglioglioma and gangliocytoma, meningioangiomatosis, lead encephalopathy, tuberous sclerosis complex, pantothenate kinase-associated neurodegeneration, neuronal ceroid lipofuscinosis, myotonic dystrophy, Fukuyama congenital muscular dystrophy, hemimegalencephaly, focal cortical dysplasias, Wolcott-Rallison syndrome, primary lateral sclerosis, progressive gait freezing, PSP with parkinsonism, Richardson's syndrome, non-fluent/agrammatic variant of primary progressive aphasia, semantic variant of primary progressive aphasia, logopenic variant of primary progressive aphasia, primary progressive apraxia of speech, amnestic Alzheimer's disease.

More preferably Alzheimer's disease (AD), Pick's disease (PiD), progressive supranuclear palsy (PSP), corticobasal degeneration (CBD), chronic traumatic encephalopathy (CTE), argyrophilic grain disease (AGD), frontotemporal dementia (FTD), amytrophic lateral sclerosis (ALS), Parkinson's disease (PD) and primary age-related tauopathy (PART).

### Pharmaceutical Compositions and Dosage

Pharmaceutical compositions of the present invention comprise the compound and at least one pharmaceutically acceptable carrier for use in preventing or treating a tauopathy.

“Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include aqueous hydroxypropyl methylcellulose, low-substituted hydroxypropyl cellulose, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of aqueous hydroxypropyl methylcellulose, low-substituted hydroxypropyl cellulose, mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In one embodiment, the pharmaceutical composition is a tablet comprising mannitol, microcrystalline cellulose, low-substituted hydroxypropyl cellulose, sodium stearyl fumarate. Preferably, the coating of the tablet comprises polyvinyl alcohol, titanium dioxide, macrogol 3350, talc, yellow iron oxide (E 172), red iron oxide (E 172), black iron oxide.

Preferably, the pharmaceutical composition is applied parenterally or orally, preferably orally.

Generally, out of 100% (for the pharmaceutical formulations/compositions), the amount of active ingredient (in particular, the amount of the compound of the present invention, optionally together with other therapeutically active agents, if present in the pharmaceutical formulations/compositions) will range from about 0.01% to about 99%, preferably from about 0.1% to about 70%, most preferably from about 1% to about 30%, wherein the reminder is preferably composed of the one or more pharmaceutically acceptable excipients.

The amount of active ingredient, e.g., a compound of the invention, in a unit dosage form and/or when administered to an individual or used in therapy, may range from about 0.1 mg to about 400 mg (for example, from about 1 mg to about 400 mg, such as from about 10 mg to about 90 mg or from about 40 mg to 80 mg or from about 80 mg to 400 mg) per unit, administration or therapy. In certain embodiments, a suitable amount of such active ingredient may be calculated using the mass or body surface area of the individual, including amounts of between about 1 mg/kg and 10 mg/kg (such as between about 2 mg/kg and 5 mg/kg), or between about 1 mg/m² and about 400 mg/m² (such as between about 3 mg/m² and about 350 mg/m² or between about 10 mg/m² and about 200 mg/m²).

In one embodiment, the pharmaceutical composition does comprise further therapeutically active components, such as in a combination therapy. In one embodiment, the pharmaceutical composition does not comprise an allosteric EGFR (epidermal growth factor receptor) inhibitor.

### EXAMPLES OF THE INVENTION

### Chemical Synthesis, In Vitro Assay, and Biological Assay Procedures

The following abbreviations may be used:
DMSO—dimethylsulfoxide
NMR—nuclear magnetic resonance
HMQC—heteronuclear multiple quantum coherence
TOCSY—total correlation spectroscopy
NOESY—nuclear Overhauser effect spectroscopy
STD—saturation transfer difference
ThT—Thioflavin T
TCEP—(tris(2-carboxyethyl)phosphine)
HEK—human embryonic kidney
GFP—green fluorescent protein
SDS-PAGE—sodium dodecyl sulfate polyacrylamide gel electrophoresis
TFA—trifluoroacetic acid
DIPEA—N,N-diisopropylethylamine
FCC—flash column chromatography
THF—tetrahydrofuran
DMF—N,N-dimethylformamide

### Synthesis/Preparation of Compounds

N-[2-(2-Dimethylaminoethylmethylamino)-4-methoxy-5-[[4-(1-methylindol-3-yl)pyrimidin-2-yl]amino]phenyl]prop-2-enamide (1) is referred to as “Osimertinib” and N-[2-(2-Dimethylaminoethylmethylamino)-4-methoxy-5-[[4-(1-methylindol-3-yl)pyrimidin-2-yl]amino]phenyl]prop-2-enamide Mesylate Salt is referred to as “Osimertinib Mesylate”. The syntheses of Osimertinib and Osimertinib Mesylate are reported in J. Med. Chem. (2014), 57, 8249-8267. Osimertinib was purchased from LC Laboratories (Massachusetts, USA, Catalog number 0-7200) and Osimertinib Mesylate from MedChemExpress (Catalog number HY-15772A).

4-methoxy-5-[[4-(1-methyl-1H-indol-3-yl)-2-pyrimidinyl]amino]-2-[methyl[2-(methylamino)ethyl]amino]phenyl]-2-Propenamide (2) is referred to as “AZ7550”, N-[4-methoxy-5-[4-(1-methyl-1H-indol-3-yl)-2-pyrimidinyl]amino]-2-[methyl[2-(methylamino)ethyl]amino]phenyl]-2-Propenamide hydrochloride (2) salt and N-[4-methoxy-5-[[4-(1-methyl-1H-indol-3-yl)-2-pyrimidinyl]amino]-2-[methyl[2-(methylamino)ethyl]amino]phenyl]-2-Propenamide (2) mesylate salt. Compound 2 is formed in vivo as a metabolite of Osimertinib (Reference: J. Med. Chem. 2014, 57, 8249-8267). The hydrochloride of compound 2 (Catalog number HY-B0794A) and the Mesylate of compound 2 (Catalog number HY-B0794B) were purchased from MedChemExpress.

N-[2-[[2-(dimethylamino)ethyl]methylamino]-5-[[4-(1H-indol-3-yl)-2-pyrimidinyl]amino]-4-methoxyphenyl]-2-Propenamide (3). Compound 3 was purchased from MedChemExpress (Catalog number HY-B0793).

N-[5-[[4-(1-cyclopropyl-1H-indol-3-yl)-2-pyrimidinyl]amino]-2-[[2-(dimethylamino)ethyl]methylamino]-4-methoxyphenyl]-2-propenamide (13), was purchased from Hoelzel Biotech (Germany, Catalog Number HS-10296, CAS Number 1899921-05-1).

N-[2-[[2-(dimethyloxidoamino)ethyl]methylamino]-4-methoxy-5-[[4-(1-methyl-1H-indol-3-yl)-2-pyrimidinyl]amino]phenyl-2-propenamide (14), was purchased from Biozol Diagnostika (Germany, Catalog Number 0702275, CAS Number 1975982-94-5).

N-[2-[[2-(dimethylamino)ethyl]methylamino]-5-[[4-(7-fluoro-1-methyl-1H-indol-3-yl)-2-pyrimidinyl]amino]-4-methoxyphenyl]-2-propenamide (4). CAS number 1883592-81-1.

N-[2-[[2-(dimethylamino)ethyl]methylamino]-5-[[4-(5-fluoro-1-methyl-1H-indol-3-yl)-2-pyrimidinyl]amino]-4-methoxyphenyl]-2-Propenamide (5). CAS number 1903753-67-2.

The syntheses of Compounds 4 and 5 are described in Bioinorganic & Medicinal Chemistry (2017), 25, 1, 4553-4559.

N-[2-[[2-(dimethylamino)ethyl]methylamino]-4-methoxy-5-[[4-(1-methyl-1H-pyrrolo[3,2-b]pyridin-3-yl)-2-pyrimidinyl]amino]phenyl]-2-propenamide (6) CAS Number 2050521-74-7.

The synthesis of Compound 6 is described in Bioorganic & Medicinal Chemistry (2018), 26, 23-24, 6135-6145.

N-[2-[[2-(dimethylamino)ethyl]methylamino]-4-methoxy-5-[[4-(3-methyl-1H-indol-1-yl)-2-pyrimidinyl]amino]phenyl]-2-propenamide (21) CAS Number 1835666-87-9. The synthesis of Compound 21 is described in European Journal of Medicinal Chemistry (2017), 135, 12-23.

### Synthesis of Compounds 7, 8, 27, and 28

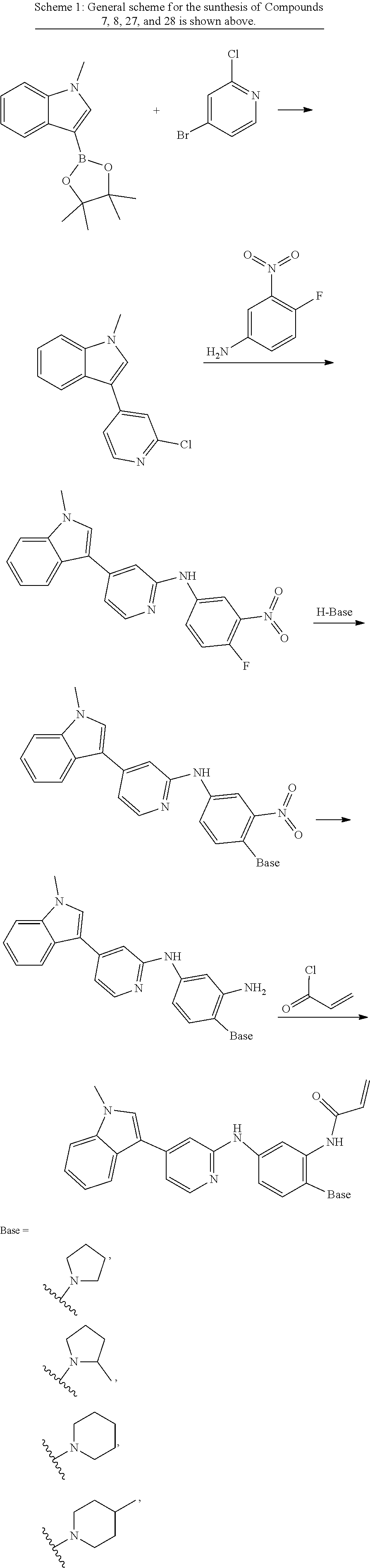

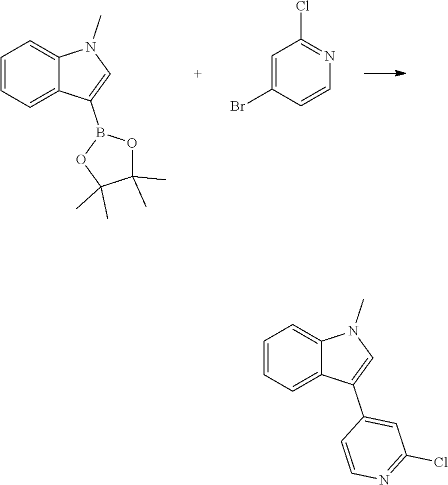 [SMILES: Cn1cc(B2OC(C)(C)C(C)(C)O2)c2ccccc21 ; Clc1cc(Br)ccn1 ; Cn1cc(-c2ccnc(Cl)c2)c2ccccc21]

In the first step, a mixture of 1-methyl-3-(4,4,5,5-tetramethyl-1,3,2-dioxaborolan-2-yl)-1H-indole and 4-bromo-2-chloropyridine in toluene is added to an aqueous mixture of tripotassium phosphate with the catalyst tetrakis(triphenylphosphine)palladium and incubated overnight. The crude product is evaporated to dryness, bound to silica powder, then purified by flash silica chromatography with a solvent gradient of 0-20% methanol in dichloromethane. The pure fraction containing with 3-(2-chloropyridin-4-yl)-1-methyl-1H-indole is evaporated to dryness.

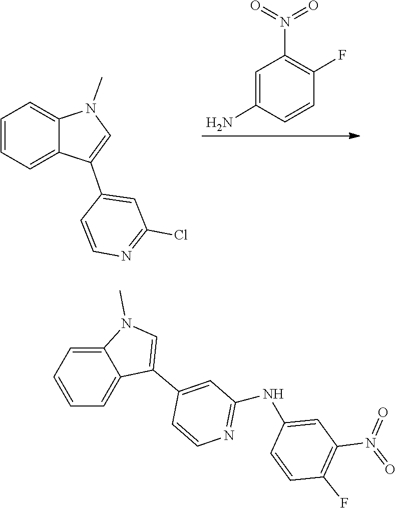

Subsequently 3-(2-chloropyridin-4-yl)-1-methyl-1H-indole is combined with an equimolar amount of 4-fluoro-3-nitroaniline dissolved in 2-pentanol. 4-methylbenzenesulfonic acid is added to the mixture, followed by heating to 105° C. for several hours. The mixture is cooled to room temperature, and the crude product is washed with 2-pentanol and subjected to flash silica chromatography or recrystallization to yield a pure fraction of N-(4-fluoro-3-nitrophenyl)-4-(1-methyl-1H-indol-3-yl)pyridine-2-amine.

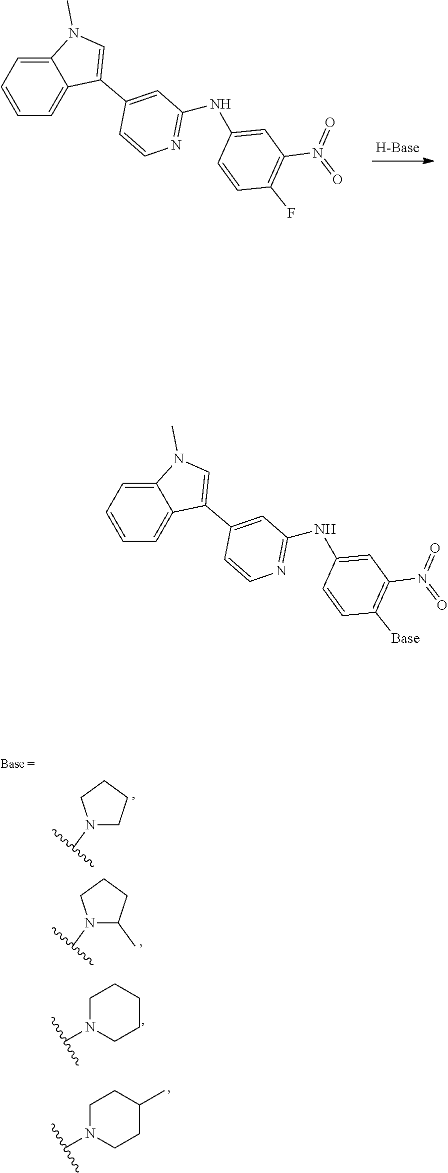

Any of the amine base precursors (“H-Base”) selected from pyrrolidine, 2-methylpyrrolidine, piperidine, or 4-methylpiperidine is added to a suspension of N-(4-fluoro-3-nitrophenyl)-4-(1-methyl-1H-indol-3-yl)pyridine-2-amine with N,N-diisopropylethylamine (DIPEA) in 2,2,2-trifluoroethanol. “Base” corresponds to pyrrolidinyl, piperidinyl, 4-methylpiperidinyl, or 2-methylpyrrolidinyl substituents. The mixture is heated in a microwave at 140° C. for one hour, after which the crude product is cooled to room temperature and subjected to ion exchange chromatography. Afterwards, flash silica chromatography is performed using a solvent gradient of 0-4% 7 M ammonia/methanol in dichloromethane. Pure fractions containing the desired base-substituted nitrobenzene ring are evaporated to dryness.

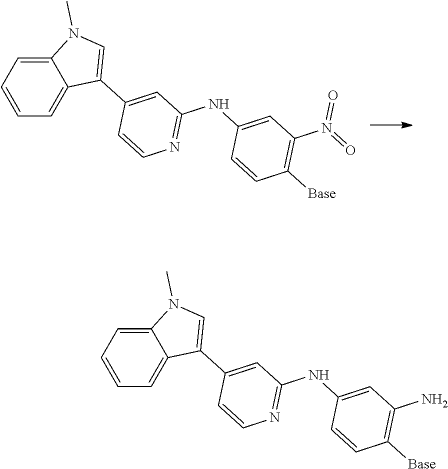

The base-substituted nitrobenzene intermediate is dissolved in ethanol/water and refluxed in the presence of ammonium chloride for 2 hours. The crude product is subjected to ion exchange chromatography followed by flash silica chromatography using an elution solvent (7 M ammonia/methanol). The pure fractions containing the desired base-substituted aniline products are evaporated to dryness.

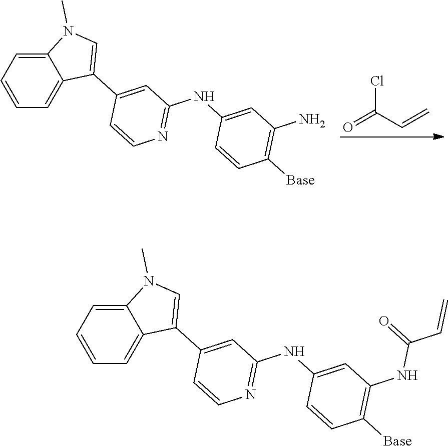

In the final reaction step, acryloyl chloride in dichloromethane is added to a stirred solution of a base-substituted aniline compound dissolved in dichloromethane containing DIPEA, cooled to 4° C. The reaction is carried out for 1.5 hours and quenched by 5-fold dilution with dichloromethane followed by washing with saturated aqueous sodium bicarbonate. The resulting hydrophobic (organic) layer is separated from the aqueous layer and subjected to silica flash chromatography, with a solvent gradient of 0-4% 7M ammonia/methanol in dichloromethane. Pure fractions are evaporated to dryness. The compounds have the following formula:

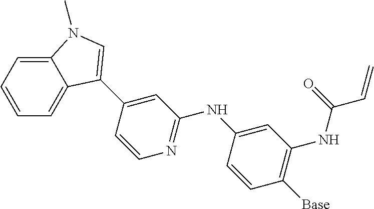

For Compound 27, “Base” is pyrrolidinyl. For Compound 7, “Base” is piperidinyl. For Compound 28, “Base” is 4-methylpiperidinyl. For Compound 8, “Base” is 2-methylpyrrolidinyl.

### Synthesis of Compounds 17, 18, 29, 30

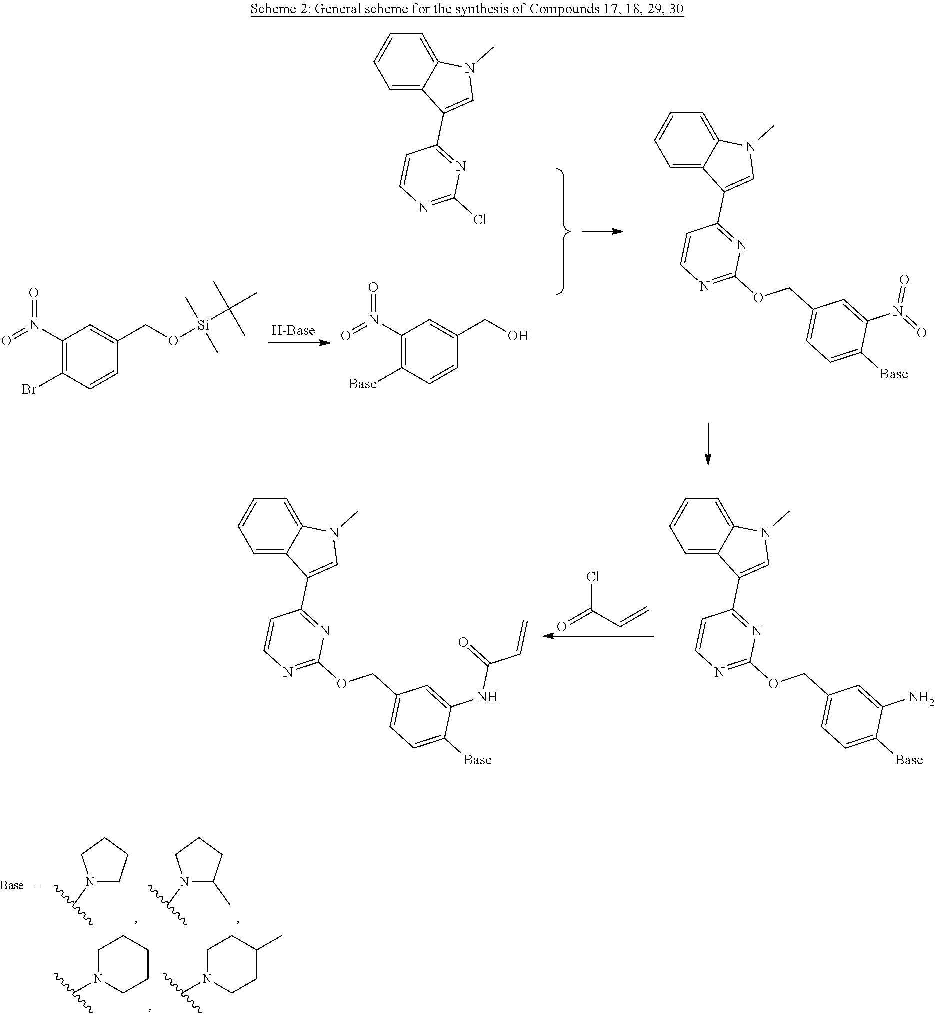

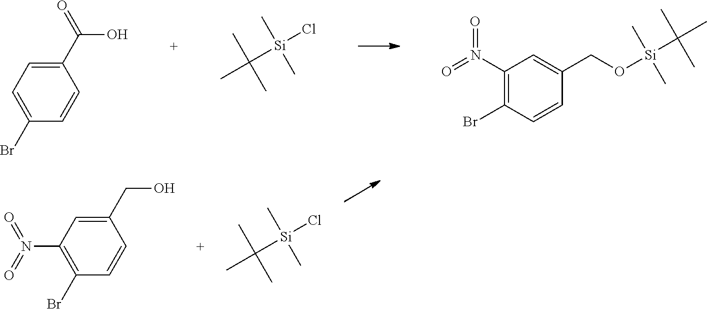

The intermediate 1-bromo-4-[[[(1,1-dimethylethyl)dimethylsilyl]oxy]methyl]-2-nitrobenzene is prepared by reacting 4-bromobenzoic acid with (1,1-dimethylethyl)dimethylsilylchloride as described in Bioconjugate Chemistry 2020, 31(2), 224-228. Alternatively, the compound may be prepared by adding (1,1-dimethylethyl)dimethylsilylchloride to a mixture of (4-bromo-3-nitrophenyl)methanol and imidazole in dichloromethane. The reaction is carried out overnight at room temperature, after which the desired product is purified by flash silica chromatography.

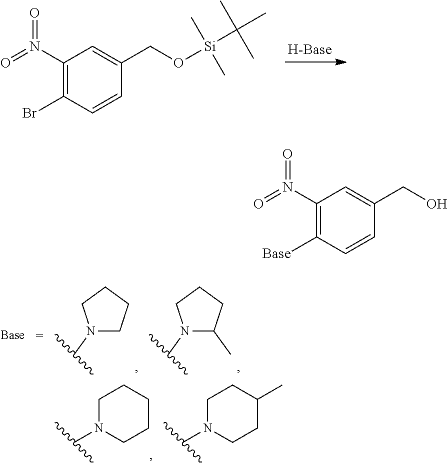

The base-substituted nitrophenylmethanol intermediate is prepared by reacting any of the amine base precursors (“H-Base”) selected from pyrrolidine, 2-methylpyrrolidine, piperidine with 1-bromo-4-[[[(1,1-dimethylethyl)dimethylsilyl]oxy]methyl]-2-nitrobenzene and DIPEA in 2,2,2-trifluoroethanol solvent at 140° C. for one hour. The crude product is subjected to flash silica chromatography and the pure fractions are evaporated to dryness.

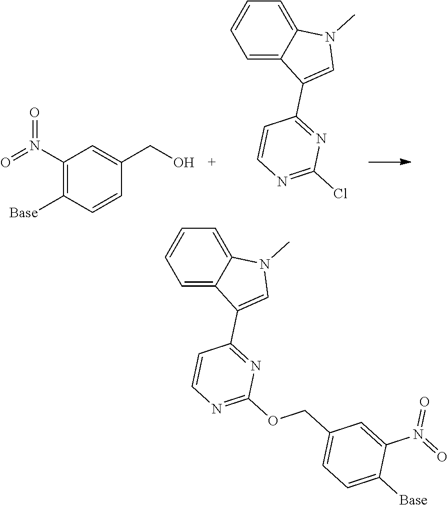

The base-substituted nitrophenylmethanol intermediate is reacted with commercially available 3-(2-chloropyrimidin-4-yl)-1-methyl-1H-indole (CAS Number 1032452-86-0) in the presence of cesium carbonate. The reaction is performed in dimethylformamide solvent for 5 hours at 100° C. The crude product is cooled to room temperature and subjected to flash silica chromatography to yield the desired fractions that bear the —OCH₂— linkage between the pyrimidine ring and the nitrobenzene ring.

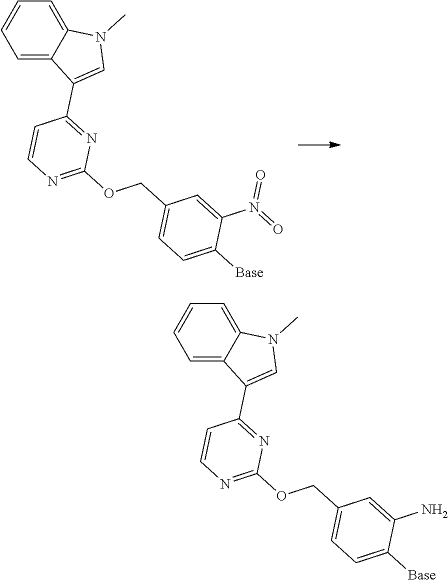

The —OCH₂-linked, base-substituted nitrobenzene intermediate is dissolved in ethanol/water and refluxed in the presence of ammonium chloride for 2 hours. The crude product is subjected to ion exchange chromatography followed by flash silica chromatography using an elution solvent (7 M ammonia/methanol). The pure fractions containing the desired products (—OCH₂-linked, base-substituted aniline compounds) are evaporated to dryness.

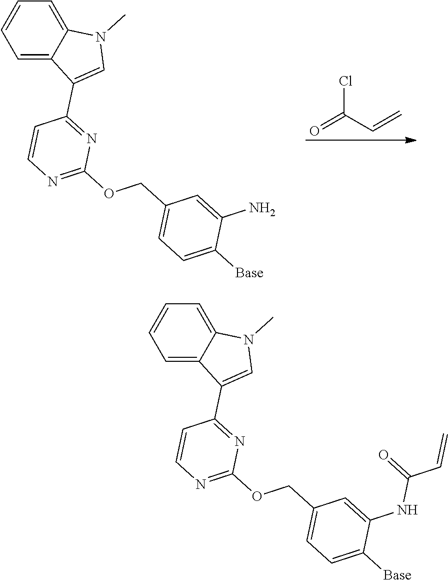

In the final reaction step, acryloyl chloride in dichloromethane is added to a stirred solution of the —OCH₂-linked, base-substituted aniline intermediate dissolved in dichloromethane containing DIPEA, cooled in an ice/water bath. The reaction is carried out for 1.5 hours and quenched by 5-fold dilution with dichloromethane followed by washing with saturated aqueous sodium bicarbonate. The resulting hydrophobic (organic) layer is separated from the aqueous layer and subjected to silica flash chromatography, with a solvent gradient of 0-4% 7M ammonia/methanol in dichloromethane. Pure fractions are evaporated to dryness. The compounds have the following formula:

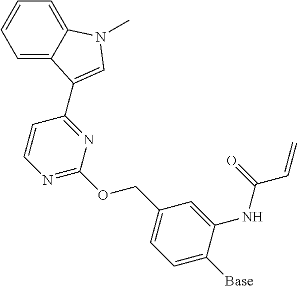

For Compound 17, “Base” is pyrrolidinyl. For Compound 18, “Base” is piperidinyl. For Compound 29, “Base” is 4-methylpiperidinyl. For Compound 30, “Base” is 2-methylpyrrolidinyl.

### Synthesis of Compounds 31, 32, 19, 20

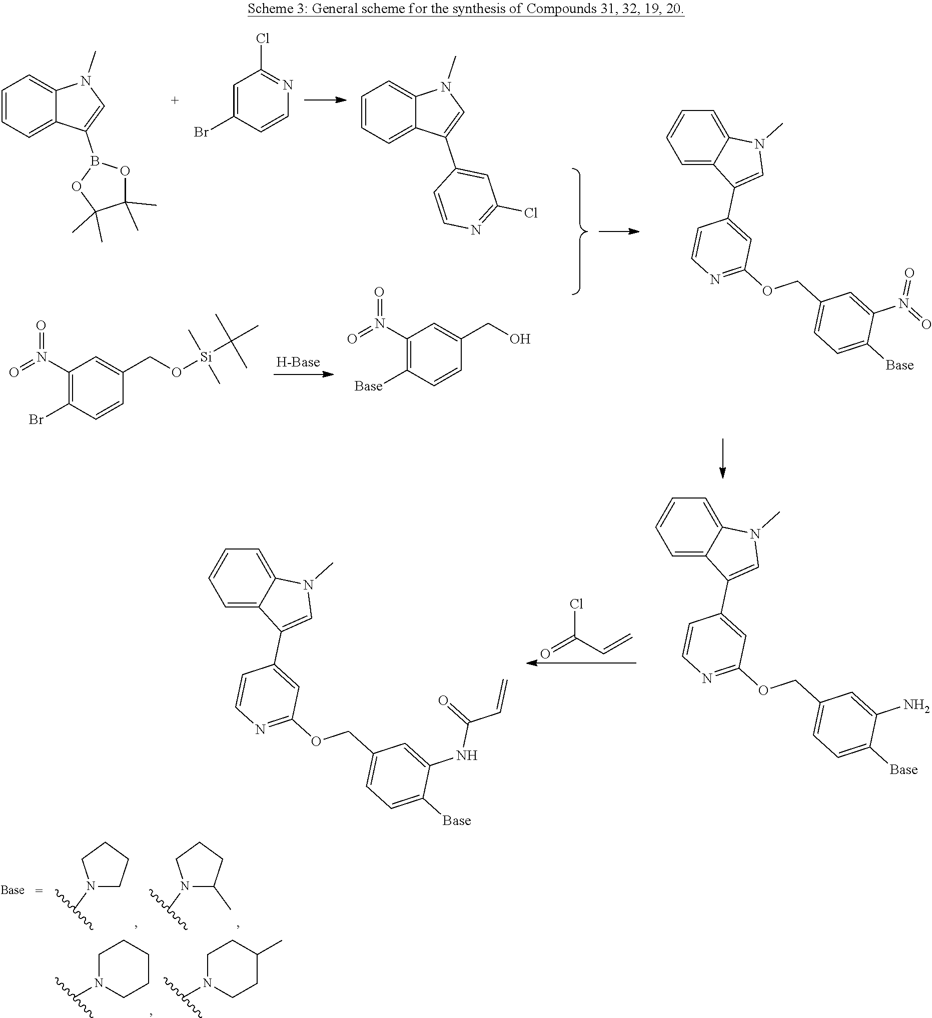

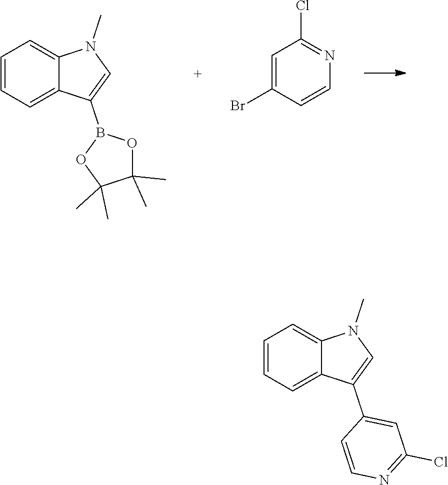 [SMILES: Cn1cc(B2OC(C)(C)C(C)(C)O2)c2ccccc21 ; Clc1cc(Br)ccn1 ; Cn1cc(-c2ccnc(Cl)c2)c2ccccc21]

A mixture of 1-methyl-3-(4,4,5,5-tetramethyl-1,3,2-dioxaborolan-2-yl)-1H-indole and 4-bromo-2-chloropyridine in toluene is added to an aqueous mixture of tripotassium phosphate with the catalyst tetrakis(triphenyl phosphine)palladium and incubated overnight. The crude product is evaporated to dryness, bound to silica powder, then purified by flash silica chromatography with a solvent gradient of 0-20% methanol in dichloromethane. The pure fraction containing with 3-(2-chloropyridin-4-yl)-1-methyl-1H-indole is evaporated to dryness.

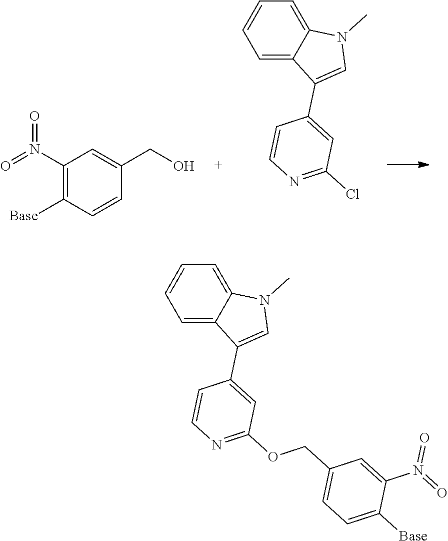

The base-substituted nitrophenylmethanol intermediate is prepared using the first two reactions described under Scheme 2. The base-substituted nitrophenylmethanol intermediate is reacted with 3-(2-chloropyridin-4-yl)-1-methyl-1H-indole in the presence of cesium carbonate. The reaction is performed in dimethylformamide solvent for 5 hours at 100° C. The crude product is cooled to room temperature and subjected to flash silica chromatography to yield the desired fractions that bear the —OCH₂— linkage between the pyridine ring and the nitrobenzene ring.

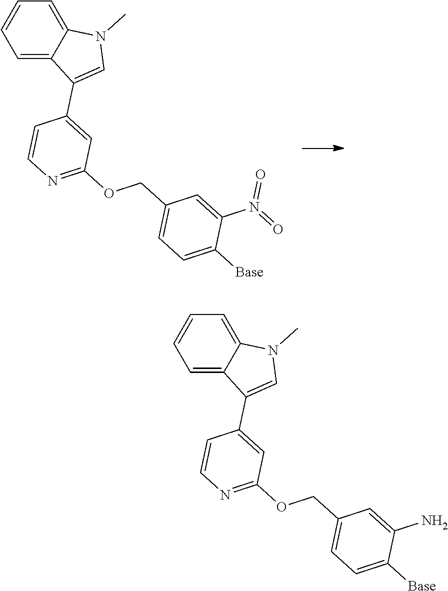

The —OCH₂-linked, base-substituted nitrobenzene intermediate is dissolved in ethanol/water and refluxed in the presence of ammonium chloride for 2 hours. The crude product is subjected to ion exchange chromatography followed by flash silica chromatography using an elution solvent (7 M ammonia/methanol). The pure fractions containing the desired products (—OCH₂-linked, base-substituted aniline compounds) are evaporated to dryness.

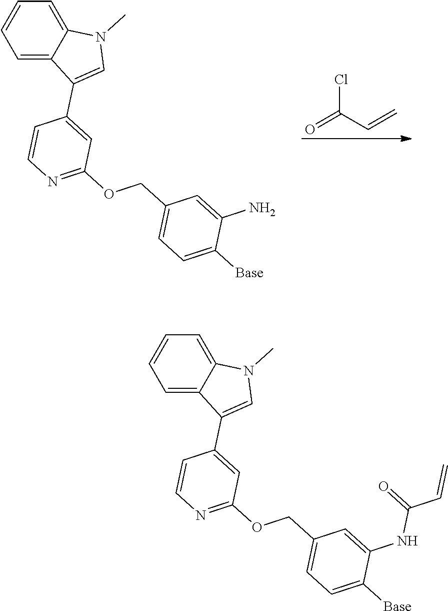

In the final reaction step, acryloyl chloride in dichloromethane is added to a stirred solution of the —OCH₂-linked, base-substituted aniline intermediate dissolved in dichloromethane containing DIPEA, cooled to 4° C. The reaction is carried out for 1.5 hours and quenched by 5-fold dilution with dichloromethane followed by washing with saturated aqueous sodium bicarbonate. The resulting hydrophobic (organic) layer is separated from the aqueous layer and subjected to silica flash chromatography, with a solvent gradient of 0-4% 7M ammonia/methanol in dichloromethane. Pure fractions are evaporated to dryness. The compounds have the following formula:

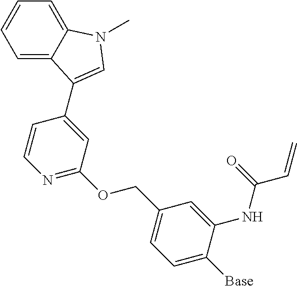

For Compound 31, “Base” is pyrrolidinyl. For Compound 32, “Base” is piperidinyl. For Compound 19, “Base” is 4-methylpiperidinyl. For Compound 20, “Base” is 2-methylpyrrolidinyl.

### NMR Spectroscopy

Stocks of compounds for NMR experiments were prepared by dissolving the powder directly in NMR buffer (50 mM sodium phosphate buffer at pH 6.8, 0.01% NaN₃) or as 10 mM compound in deuterated DMSO.

To obtain residue-specific information about the interaction between Tau and the compounds, ¹H-¹⁵N correlation spectra of 2N4R Tau were monitored over the course of a titration with increasing amounts of compound. The ¹H-¹⁵N SOFAST-HMQC (Reference: Schanda, P., Kupce, E. & Brutscher, B. SOFAST-HMQC experiments for recording two-dimensional heteronuclear correlation spectra of proteins within a few seconds. J. Biomol. NMR 33, 199-211 (2005)) spectra of [U-¹⁵N]-Tau were acquired at 5° C. on a Bruker 800 MHz spectrometer equipped with a triple-resonance cryoprobe. Tau samples (18-25 μM) were prepared in NMR buffer containing 10% (v/v) D₂O, and with compound concentrations that corresponded to Tau:compound mole ratios of 1:10 and 1:100. All NMR samples were incubated overnight (˜16 h) at 37° C. prior to spectral acquisition. Tau amide assignments were taken from a previous study (Reference: Mukrasch, M. et al. Structural polymorphism of 441-residue tau at single residue resolution. PLoS Biol. 7, e34 (2009)). However, because previous assignments were obtained using a Tau sample prepared in the presence of reducing agents, some amide resonances belonging to the repeat region of Tau (close to C291 and C322) were not unambiguously transferred to the SOFAST-HMQC spectra acquired under oxidizing conditions. Therefore, to obtain nearly complete backbone assignments in the repeat region, 3D HNCA, 3D HNCOCA, and 3D CBCACONH spectra were recorded for a [U-¹³C,¹⁵N]-4R Tau (K18 construct) sample prepared under the same buffer condition. The weighted, normalized chemical shift perturbations (CSP) were calculated as CSP=√[0.5 [(Δδ H)²+(Δδ N)²/25]. NMR signal intensity ratios, I/I₀ (where I is the peak intensity of Tau amide backbone resonances in the presence of the compounds, and I₀ is the peak intensity in the absence of the compounds), were calculated for each titration point.

To obtain assignments of the non-labile protons in Osimertinib, AZ7550 hydrochloride, and AZ5104 (250 μM compound in 50 mM sodium phosphate at pH 6.8 with 2.5% v/v deuterated DMSO), the following NMR experiments were performed for each compound: 1D ¹H-NMR, 2D ¹H-¹H TOCSY with 80 ms mixing time, and 2D ¹H-¹H NOESY with 200 ms mixing time. Experiments were performed at 25° C. and 37° C. using Bruker 800 MHz or Bruker 600 MHz spectrometers equipped with triple resonance cryoprobes.

To determine binding epitopes of the compounds for monomeric 2N4R Tau, 1D ¹H-NMR titrations were performed in which the spectra of Osimertinib, AZ7550 hydrochloride, and AZ5104 (250 μM compound) were recorded with unlabelled 2N4R Tau (0, 10 and 20 μM). The NMR titration samples were prepared in 50 mM sodium phosphate at pH 6.8 with 2.5% v/v deuterated DMSO. NMR spectra were recorded at 37° C. using a Bruker 800 MHz spectrometer equipped with a triple resonance cryoprobe.

To determine binding epitopes of the compounds for fibrillar Tau, 1D saturation-transfer difference (STD) experiments were performed with Osimertinib, AZ7550 hydrochloride, and AZ5104 in the presence of sonicated 2N4R Tau fibrils (circa 100 nm fibril length). 2N4R Tau fibrils were prepared using the aggregation procedure described in the section in vitro assay 1 without Thioflavin T. The mole ratio of “free” compound to fibrils was 48:1 (corresponding to a total compound concentration of 250 μM and a total fibril concentration of 5 μM Tau). The NMR samples were prepared in 50 mM sodium phosphate with 100 mM NaCl and 2.5% (v/v) DMSO at pH 6.8. The spectra were recorded at 37° C. using a Bruker 700 MHz spectrometer equipped with a triple resonance cryoprobe. 1D ¹H-NMR STD spectra were acquired in an interleaved manner, alternating between off-resonance irradiation at 60 ppm and on-resonance irradiation at −2 ppm.

NMR spectra were processed using Topspin 3.6.2 (Bruker) and analyzed using NMRFAM-Sparky (Reference: Bioinformatics. 2015 Apr. 15; 31(8):1325-7. Epub 2014 Dec. 12 NMRFAM-SPARKY: enhanced software for biomolecular NMR spectroscopy).

### In Vitro Assay Procedures

### In Vitro Assay 1: De Novo and Seeded Tau Aggregation Assays

Aggregation of 2N4R Tau was performed using a previously published co-factor-free aggregation protocol, with a few modifications (Reference: Chakraborty, P. et al. Co-factor-free aggregation of tau into seeding-competent RNA-sequestering amyloid fibrils. Nat. Commun. 12, 4231 (2021)). Aggregation experiments used the Tau protein stock prepared in 25 mM HEPES pH 7.4, 1 mM TCEP. Stocks of compounds were prepared in aggregation buffer (25 mM HEPES, 10 mM KCl, 5 mM MgCl₂, pH 7.2) or in DMSO (10 mM compound).

To prepare the Tau fibril seeds by de novo aggregation, 2N4R Tau at a monomer concentration of 25 μM was diluted in aggregation buffer. The working concentration of TCEP in the aggregation mixture was <25 μM after dilution of the stock. To this mixture, Thioflavin T (ThT) was added to a final concentration of 50 μM. A total volume of 100 μL of the Tau-ThT mixture was incubated in a well of a 96-well microplate (Greiner Bio-one), in the presence of two polytetrafluoroethylene beads. The microplate was subjected to a program implemented in a Tecan Spark plate reader that involved incubating at 37° C., with intervals of double orbital shaking and ThT fluorescence emission measurement. The excitation wavelength for ThT was set as 430 nm and the emission wavelength was 485 nm. Details of this plate reader program were published previously (Reference: Chakraborty, P. et al. Co-factor-free aggregation of tau into seeding-competent RNA-sequestering amyloid fibrils. Nat. Commun. 12, 4231 (2021)). After about 3-6 days of incubation, ThT fluorescence emission reached a maximal value suggesting that Tau fibrillization had reached a saturation point. The fibril-containing mixture collected from the microplate served as a seeding mixture for the subsequent aggregation assays.

Seeded aggregation assays were performed as described above but with 1.0% of the well volume occupied by the seeding mixture sonicated for 1 minute. The assays were performed in the presence and absence of compounds, and working concentrations ranged from 0-250 μM compound.

Analyses of the fluorescence curves were performed using Graphpad PRISM version 8. Fluorescence data were fit to a sigmoid function. The number of hours corresponding to half-maximal ThT Fluorescence™ as well as the span of fluorescence intensity (Span) were determined for each sigmoid curve. Statistically significant differences were determined by one-way ANOVA analysis with Dunnett's multiple comparison test or unpaired t-test.

The described seeded aggregation protocol was likewise applied to 3R Tau and the C291S/C322S mutant of 2N4R Tau.

### In Vitro Assay 2: Tau Pelleting Assay

Aggregation assays were performed as described in the section In vitro assay 1. The aggregation mixtures were harvested and ultracentrifuged (55000 rpm using a JLA 100.3 rotor, Optima MAX-XP) for 30 minutes to separate the soluble (supernatant) and insoluble (pellet) fractions. Soluble fractions were analyzed by SDS-PAGE using a 12% or 15% acrylamide resolving gel and a 5% acrylamide stacking gel. Band intensities of soluble Tau were quantified by ImageLab (Bio-Rad Laboratories).

### Negative Stain Transmission Electron Microscopy

Fibril samples were negative stained using glow discharged 400 mesh carbon-coated copper grids. After staining using a 1% uranyl acetate solution, images were acquired on a Talos L120C transmission electron microscope (Thermo Fisher, Eindhoven, The Netherlands).

### Mass Spectrometry Procedures

### Mass Spectrometry Analysis 1: ESI-MS

This analysis was performed using the 4R Tau construct (K18 construct, 13818 Da) comprising residues 244-372 of 2N4R Tau. Compounds (330 μM) were incubated with 4R Tau (65 μM) for 16 h at 37° C. in 10 mM ammonium bicarbonate at pH 7.0. The compound/4R Tau mixtures were injected into an LC system (ACQUITY) coupled to a single quadrupole mass detection system (SQ Detector 2, Waters) with diode-array detectors (DAD). The DAD spectrum covers wavelengths of 210-400 nm. Components of the compound-4R Tau mixture were separated using a BioResolve RP mAb column (Waters) with a gradient of 5-95% buffer B (buffer A is 0.1% TFA in water and buffer B is 0.1% TFA in acetonitrile). Covalent modification of 4R Tau corresponded to mass additions of 500 Da (Osimertinib) and 486 Da (AZ7550 or AZ5104).

### Mass Spectrometry Analysis 2: MS/MS

Compounds (330 μM) were incubated with 2N4R Tau (65 μM) in buffer (25 mM HEPES, 10 mM KCl, 5 mM MgCl₂, pH 7.2) for 16 h at 37° C. then stored at 4° C. until in-gel digestion.

In-gel digestion using trypsin (Sigma Aldrich) followed by extraction of peptides for mass spectrometry were performed as described previously (Reference: Shevchenko, A., Tomas, H., Havlis, J., Olsen, J. V., and Mann, M. (2006). In-gel digestion for mass spectrometric characterization of proteins and proteomes. Nature protocols 1, 2856-2860). Extracted peptides were resuspended in 2% acetonitrile and 0.05% TFA and analyzed using a Orbitrap Exploris 480 Mass Spectrometer (Thermo Fisher). The mass spectrometer used an emitter voltage at 2100 kV and ion transfer tube temperature at 300° C. Full scans were recorded in the range 350 to 1600 m/z with a mass resolution of 15000. Peptides were fragmented in the collision cell with a 28% energy setting. Tandem mass spectra were acquired using Scan Range Mode “Define First Mass” and isolation window of 1.6 m/z with a mass resolution of 15000.

### Biological Assay Procedures

### Biological Assay 1: HEK Biosensor Cell Tau Seeding Assay

Compound stock solutions were prepared as 10 mM compound in DMSO.

HEK293T biosensor cells expressing the human tau repeat domain (RD) harboring the P301L/V337M mutation and fused to a C-terminal GFP tag (named TauRD^{LM}-GFP) were engineered to probe Tau seeding in cellula (Reference: Liu, S., Hossinger, A., Heumüller, S E. et al. Highly efficient intercellular spreading of protein misfolding mediated by viral ligand-receptor interactions. Nat Commun 12, 5739 (2021). https://doi.org/10.1038/s41467-021-25855-2). HEK cells expressing the P301L human full-length Tau, C-terminally tagged with GFP were incubated with pre-sonicated hTau₄₀^{P301L} fibrils to induce Tau aggregate formation. Cells were harvested and the resulting cell lysate was used to seed Tau aggregation in further experiments with HEK TauRD^{LM}-GFP cells.

In a typical seeded assay, HEK cells were incubated with cell lysate, compounds (0-40 μM) and lipofectamine for 22-48 hours. Negative control experiments in the absence of compounds (only with vehicle DMSO) and in the presence of cell lysate were performed in parallel.

Dose response curves were constructed by quantifying the number of cells with aggregates for each compound concentration. The curves were fitted with a sigmoid function using Graphpad PRISM version 8. The inhibitory concentration at 50% activity (IC₅₀) was determined for each compound.

### Biological Assay 2: Drosophila Neurodegeneration Model

### Drug Treatments in Drosophila:

Stock solutions of individual compounds were prepared by dissolving 10 mg of the compound in 100 μl of 100% DMSO. Osimertinib, Osimertinib mesylate and AZ7550 mesylate were orally fed to the flies by mixing them with food. The final concentration of each compound, which was used for treating the Drosophila, was derived on the basis of recommended concentrations for human use (reference concentration) based on body weight.

We have used three concentrations for each compound. One was equal to the reference concentration, and two were higher than the reference concentration. Final concentrations of different compounds mixed with fly food are: Osimertinib mesylate, 5 M, 10 M, and 15 M; Osimertinib, 10 M; AZ 7550 mesylate, 10 M and 20 M. After hatching, first instar larvae were transferred onto the food containing one of the compounds. Food mixed with drug at required concentration was replaced with fresh food every 48 h.

### Light Microscopy:

Light microscopy was performed after mounting the fly heads on the glass slide using a double-sided adhesive tape. Full-eye images were captured with the help of an Infinity Analyze camera attached to an Olympus SZX7 microscope with a halogen light source. Total eye surface area and degenerated eye surface area were marked and calculated with Infinity Analyze software (Lumenera corporation). Percentage of degenerated area was calculated and plotted using GraphPad Prism 8.

### Scanning Electron Microscopy:

After the eclosion, treated and control flies (2-3 days old) were fixed in 1% formaldehyde for two hours, followed by serial dehydration steps of 12 hrs each in 25%, 50%, 75% and 100% ethanol were carried out. Flies were stored in 100% ethanol and dried with CPD before mounting on the stab for imaging. Eye Images were captured at 150× and analyzed using Infinity Analyze software.

### REFERENCES

1. Gbejuade, Herbert O.; Lovering, Andrew M.; Webb, Jason C. (2015): The role of microbial biofilms in prosthetic joint infections. In: Acta orthopaedica 86 (2), S. 147-158. DOI: 10.3109/17453674.2014.966290.
2. Ong, Kevin L.; Kurtz, Steven M.; Lau, Edmund; Bozic, Kevin J.; Berry, Daniel J.; Parvizi, Javad (2009): Prosthetic joint infection risk after total hip arthroplasty in the Medicare population. In: The Journal of arthroplasty 24 (6 Suppl), S. 105-109. DOI: 10.1016/j.arth.2009.04.027.
3. Gosheger, Georg; Gebert, Carsten; Ahrens, Helmut; Streitbuerger, Arne; Winkelmann, Winfried; Hardes, Jendrik (2006): Endoprosthetic reconstruction in 250 patients with sarcoma. In: Clinical orthopaedics and related research 450, S. 164-171. DOI: 10.1097/01.blo.0000223978.36831.39.
4. Funovics, Philipp T.; Hipfl, Christian; Hofstaetter, Jochen G.; Puchner, Stephan; Kotz, Rainer I.; Dominkus, Martin (2011): Management of septic complications following modular endoprosthetic reconstruction of the proximal femur. In: International Orthopaedics (SICOT) 35 (10), S. 1437-1444. DOI: 10.1007/s00264-010-1054-0.
5. Theil, C.; Röder, J.; Gosheger, G.; Deventer, N.; Dieckmann, R.; Schorn, D. et al. (2019): What is the Likelihood That Tumor Endoprostheses Will Experience a Second Complication After First Revision in Patients With Primary Malignant Bone Tumors And What Are Potential Risk Factors? In: Clinical orthopaedics and related research 477 (12), S. 2705-2714. DOI: 10.1097/CORR.0000000000000955.
6. Gosheger, Georg; Sass, Jens (2002): ENDOPROTHESE MIT GALVANISCHER SILBERSCHICHT. Angemeldet durch Implantcast GmbH am 18.02.2002. Anmeldenr: 02719863. Veröffentlichungsnr: EP000001361837B1.
7. Romanò, Carlo Luca; Scarponi, Sara; Gallazzi, Enrico; Romanò, Delia; Drago, Lorenzo (2015): Antibacterial coating of implants in orthopaedics and trauma: a classification proposal in an evolving panorama. In: Journal of orthopaedic surgery and research 10, S. 157. DOI: 10.1186/s13018-015-0294-5.
8. Hardes, Jendrik; Eiff, Christof von; Streitbuerger, Arne; Balke, Maurice; Budny, Tymoteus; Henrichs, Marcel P. et al. (2010): Reduction of periprosthetic infection with silver-coated megaprostheses in patients with bone sarcoma. In: Journal of surgical oncology 101 (5), S. 389-395. DOI: 10.1002/jso.21498.
9. Schmidt-Braekling, Tom; Streitbuerger, Arne; Gosheger, Georg; Boettner, Friedrich; Nottrott, Markus; Ahrens, Helmut et al. (2017): Silver-coated megaprostheses: review of the literature. In: European journal of orthopaedic surgery & traumatology: orthopedie traumatologie 27 (4), S. 483-489. DOI: 10.1007/s00590-017-1933-9.
10. Scoccianti, Guido; Frenos, Filippo; Beltrami, Giovanni; Campanacci, Domenico Andrea; Capanna, Rodolfo (2016): Levels of silver ions in body fluids and clinical results in silver-coated megaprostheses after tumour, trauma or failed arthroplasty. In: Injury 47 Suppl 4, S11-S16. DOI: 10.1016/j.injury.2016.07.042.
11. Mair W, Muntel J, Tepper K, Tang S, Biernat J, Seeley W W, Kosik K S, Mandelkow E, Steen H, Steen J A. Anal Chem. 2016 Apr. 5; 88(7):3704-14. doi: 10.1021/acs.analchem.5b04509. Epub 2016 Mar. 7. PMID: 26877193.
12. Wesseling H, Mair W, Kumar M, Schlaffner C N, Tang S, Beerepoot P, Fatou B, Guise A J, Cheng L, Takeda S, Muntel J, Rotunno M S, Dujardin S, Davies P, Kosik K S, Miller B L, Berretta S, Hedreen J C, Grinberg L T, Seeley W W, Hyman B T, Steen H, Steen J A. Cell. 2020 Dec. 10; 183(6):1699-1713.e13. doi: 10.1016/j.cell.2020.10.029. Epub 2020 Nov. 13. PMID: 33188775.
13. Samimi N, Sharma G, Kimura T, Matsubara T, Huo A, Chiba K, Saito Y, Murayama S, Akatsu H, Hashizume Y, Hasegawa M, Farjam M, Shahpasand K, Ando K, Hisanaga S I. Neurobiol Aging. 2021 December; 108:72-79. doi: 10.1016/j.neurobiolaging.2021.08.011. Epub 2021 Aug. 21. PMID: 34536819.
14. Shin M K, Vezquez-Rosa E, Koh Y, Dhar M, Chaubey K, Cintrón-Pérez C J, Barker S, Miller E, Franke K, Noterman M F, Seth D, Allen R S, Motz C T, Rao S R, Skelton L A, Pardue M T, Fliesler S J, Wang C, Tracy T E, Gan L, Liebl D J, Savarraj J P J, Torres G L, Ahnstedt H, McCullough L D, Kitagawa R S, Choi H A, Zhang P, Hou Y, Chiang C W, Li L, Ortiz F, Kilgore J A, Williams N S, Whitehair V C, Gefen T, Flanagan M E, Stamler J S, Jain M K, Kraus A, Cheng F, Reynolds J D, Pieper A A. Cell. 2021 May 13; 184(10):2715-2732.e23. doi: 10.1016/j.cell.2021.03.032. Epub 2021 Apr. 13. PMID: 33852912.
15. Kovacs, G. G., Ghetti, B. and Goedert, M. (2022), Classification of diseases with accumulation of Tau protein. Neuropathol Appl Neurobiol, 48: e12792.
16. Rankovic M, Zweckstetter M. Neurosci Biobehav Rev. 2019 March; 98:1-9. doi: 10.1016/j.neubiorev.2018.12.014. Epub 2018 Dec. 11.
17. Guo, T., Noble, W. & Hanger, D. P. Roles of tau protein in health and disease. Acta Neuropathol 133, 665-704 (2017).
18. Strong M J, Donison N S, Volkening K. Alterations in Tau Metabolism in ALS and ALS-FTSD. Front Neurol. 2020 Nov. 23; 11:598907.
19. Pan L, Meng L, He M, Zhang Z. Tau in the Pathophysiology of Parkinson's Disease. J Mol Neurosci. 2021 November; 71(11):2179-2191.
20. Brown, L. A., Scarola, J., Smith, A. J. et al. The role of tau protein in HIV-associated neurocognitive disorders. Mol Neurodegeneration 9, 40 (2014).
21. Hart N J, Koronyo Y, Black K L, Koronyo-Hamaoui M. Ocular indicators of Alzheimer's: exploring disease in the retina. Acta Neuropathol. 2016.

1. A compound according to formula (I) for use in preventing or treating a tauopathy, 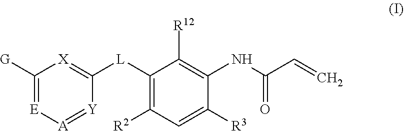
wherein
G is 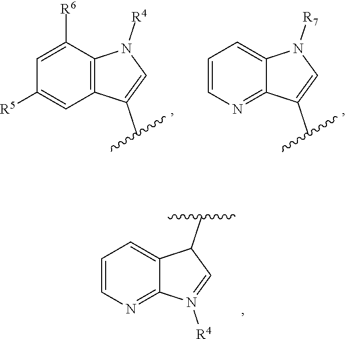
preferably 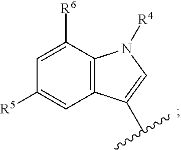
X is N or C—H, preferably N;
Y is N or C—H, preferably N;
L is 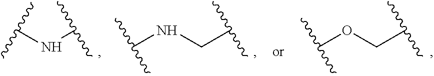
preferably 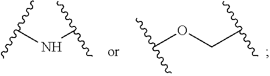
A is N or C—R; preferably C—R, more preferably CH;
E is N or C—R¹; preferably C—R¹, more preferably CH;
R is hydrogen, fluoro, chloro, bromo, —(C₁-C₆)alkyl, cyano, or —O(C₁-C₆)alkyl, preferably hydrogen;
R¹ is hydrogen, fluoro, chloro, bromo, —(C₁-C₆)alkyl, cyano, or —O(C₁-C₆)alkyl, preferably hydrogen;
R² is hydrogen, fluoro, chloro, bromo, —(C₁-C₆)alkyl, cyano, —O(C₁-C₆)alkyl, or difluoromethoxy, preferably —O(C₁-C₆)alkyl;
R³ is 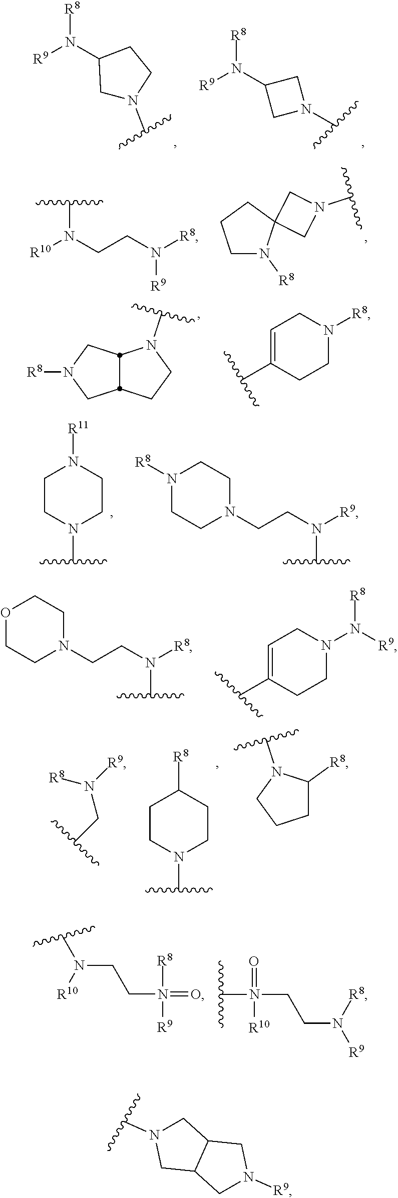
or —NR⁸R⁹, preferably 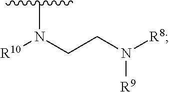
R⁴ is hydrogen, —(C₁-C₆)alkyl, or —(C₃-C₆)cycloalkyl;
R⁵ is hydrogen, fluoro, or chloro;
R⁶ is hydrogen, fluoro or chloro;
R⁷ is hydrogen, or —(C₁-C₆)alkyl;
R⁸ is hydrogen, or —(C₁-C₆)alkyl, preferably —(C₁-C₆)alkyl;
R⁹ is hydrogen, or —(C₁-C₆)alkyl, preferably —(C₁-C₆)alkyl;
R¹⁰ is hydrogen, or —(C₁-C₆)alkyl;
R¹¹ is hydrogen, —(C₁-C₆)alkyl, or —CH₂CON((C₁-C₆)alkyl)₂;
R¹² is hydrogen, or —(C₁-C₆)alkyl;
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope;
one or more fluoro atoms are replaced by the corresponding ¹⁸F isotope;
or a pharmaceutically acceptable salt thereof.

2. A compound according to formula (I) 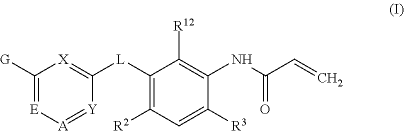
wherein
G is 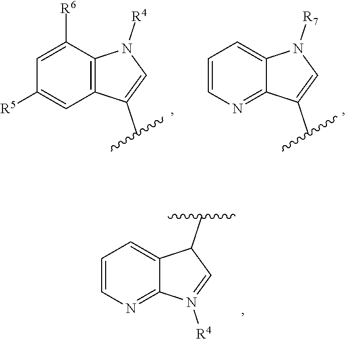
preferably 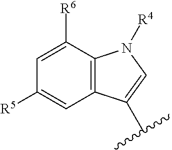
X is selected from N or C—H;
Y is selected from N or C—H;
L is 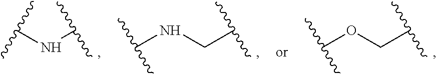
preferably 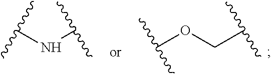
A is selected from N or C—R;
E is selected from N or C—R¹;
R is hydrogen, fluoro, chloro, bromo, —(C₁-C₆)alkyl, cyano, or —O(C₁-C₆)alkyl, preferably hydrogen;
R¹ is hydrogen, fluoro, chloro, bromo, —(C₁-C₆)alkyl, cyano, or —O(C₁-C₆)alkyl, preferably hydrogen;
R² is hydrogen, fluoro, chloro, bromo, —(C₁-C₆)alkyl, cyano, —O(C₁-C₆)alkyl, or difluoromethoxy, preferably —O(C₁-C₆)alkyl;
R³ is 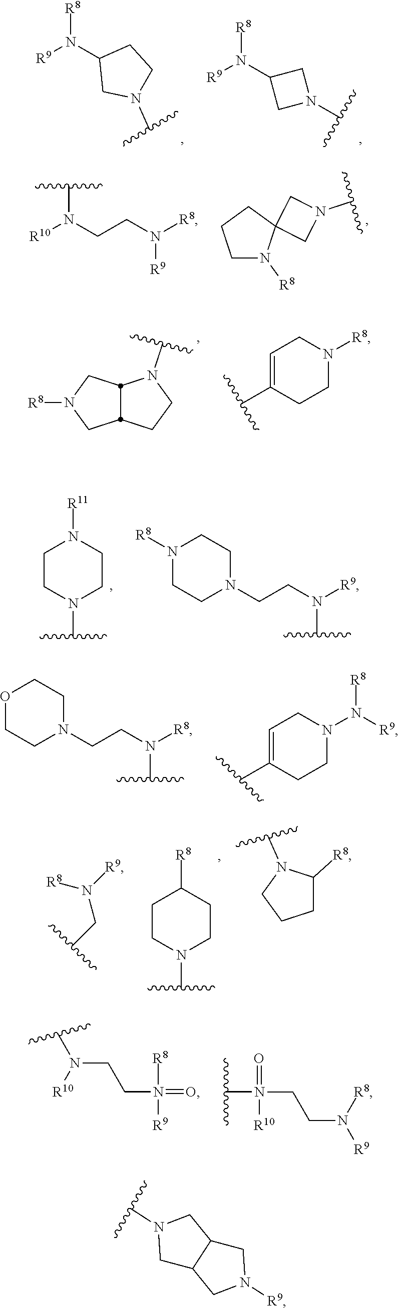
or —NR⁸R⁹, preferably 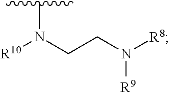
R⁴ is hydrogen, —(C₁-C₆)alkyl, or —(C₃-C₆)cycloalkyl;
R⁵ is hydrogen, fluoro, or chloro;
R⁶ is hydrogen, fluoro or chloro;
R⁷ is hydrogen, or —(C₁-C₆)alkyl;
R⁸ is hydrogen, or —(C₁-C₆)alkyl, preferably —(C₁-C₆)alkyl;
R⁹ is hydrogen, or —(C₁-C₆)alkyl, preferably —(C₁-C₆)alkyl;
R¹⁰ is hydrogen, or —(C₁-C₆)alkyl;
R¹¹ is hydrogen, —(C₁-C₆)alkyl, or —CH₂CON((C₁-C₆)alkyl)₂;
R¹² is hydrogen, or —(C₁-C₆)alkyl;
with the provision that the compound according to formula (I) is not selected from the group consisting of 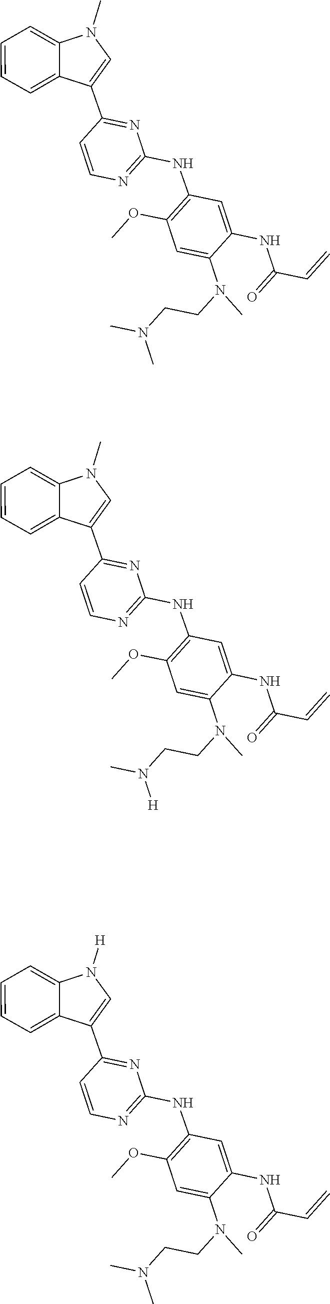 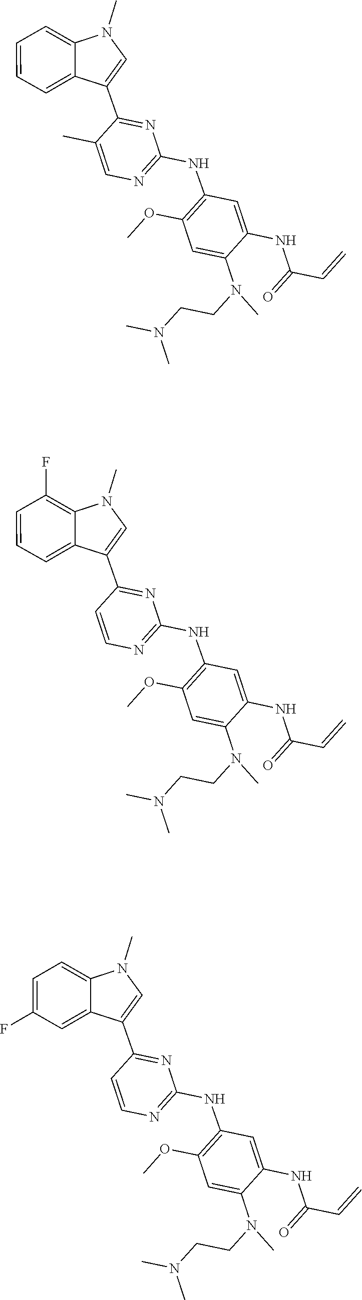 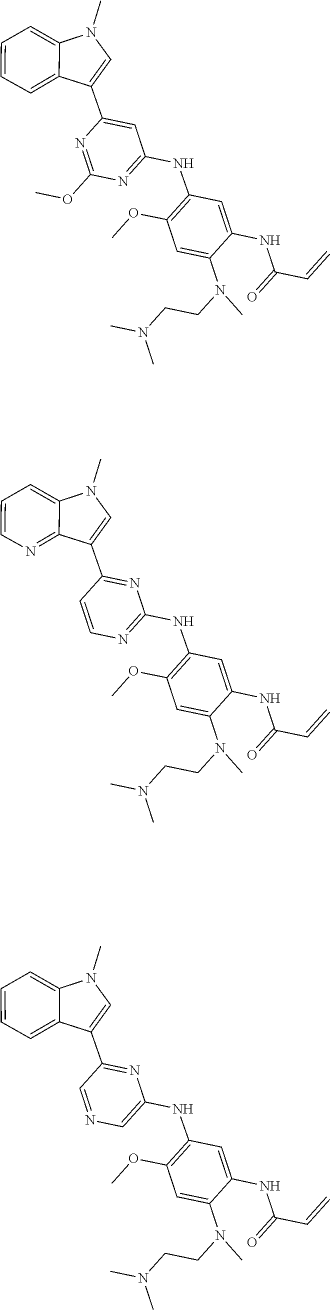 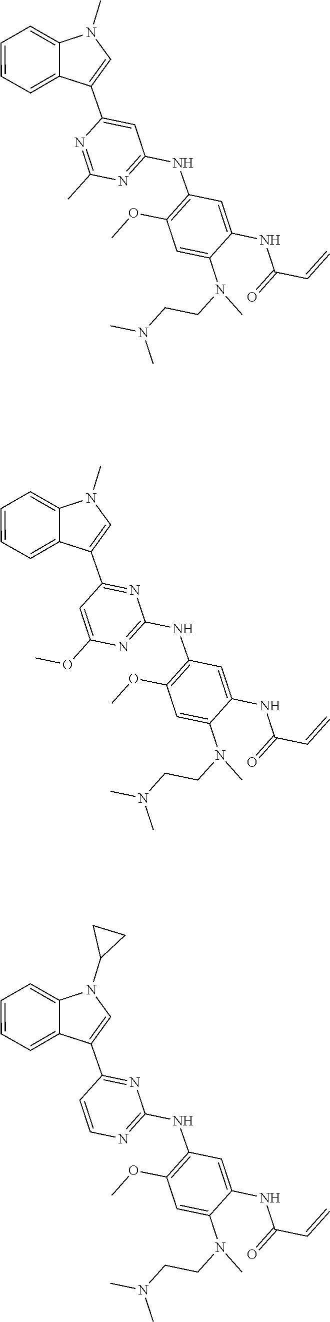 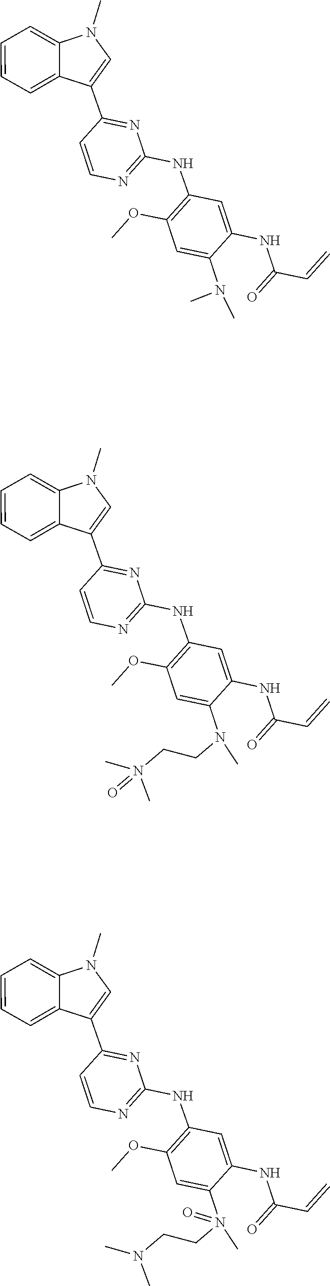 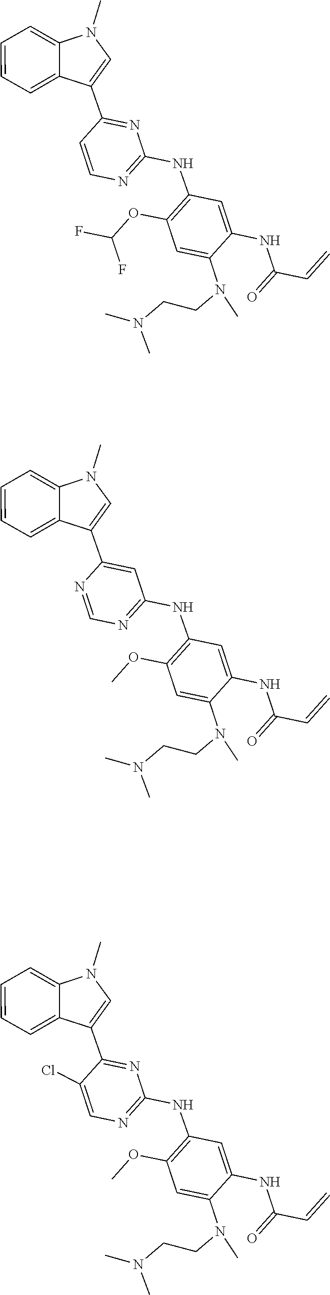 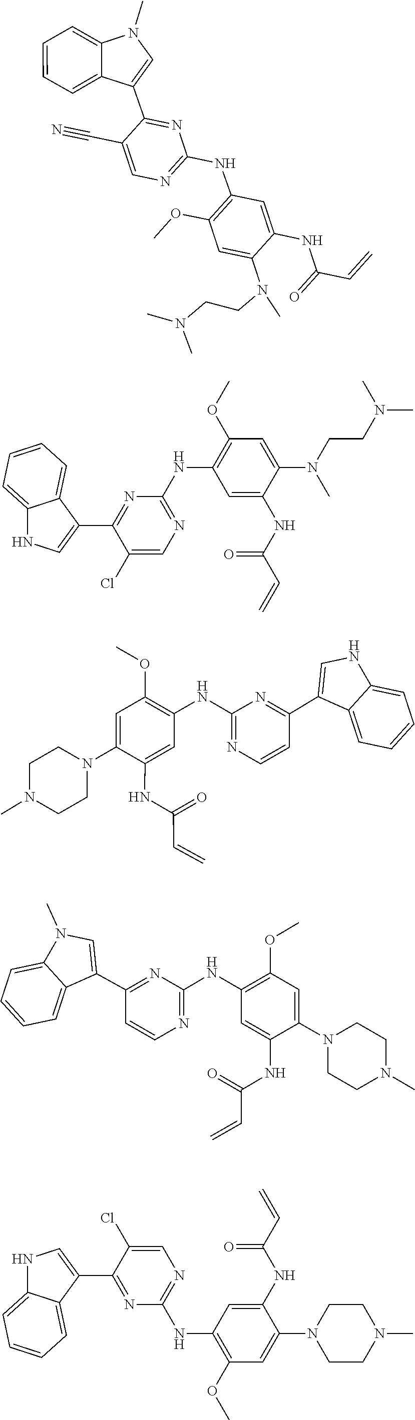 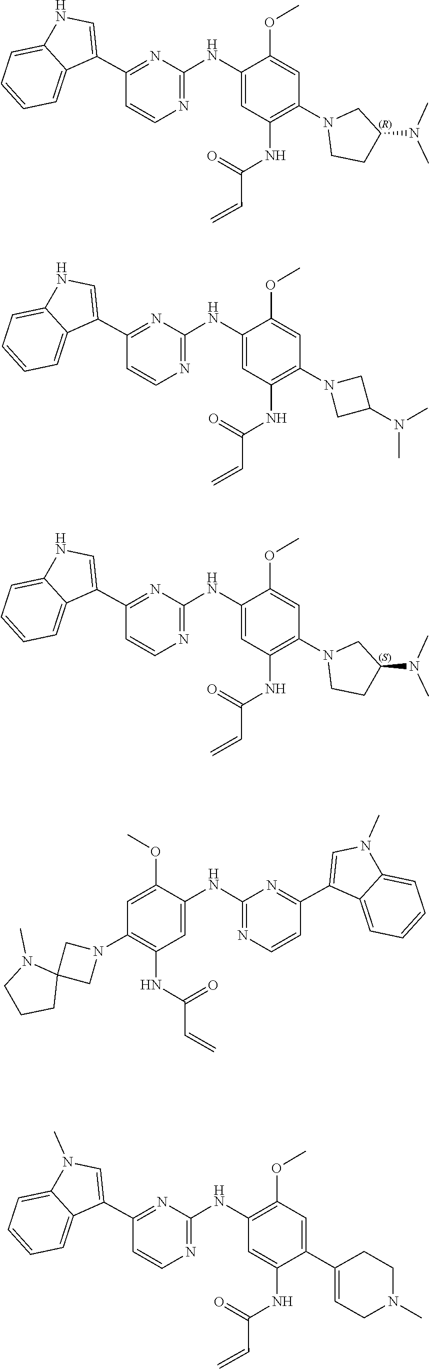 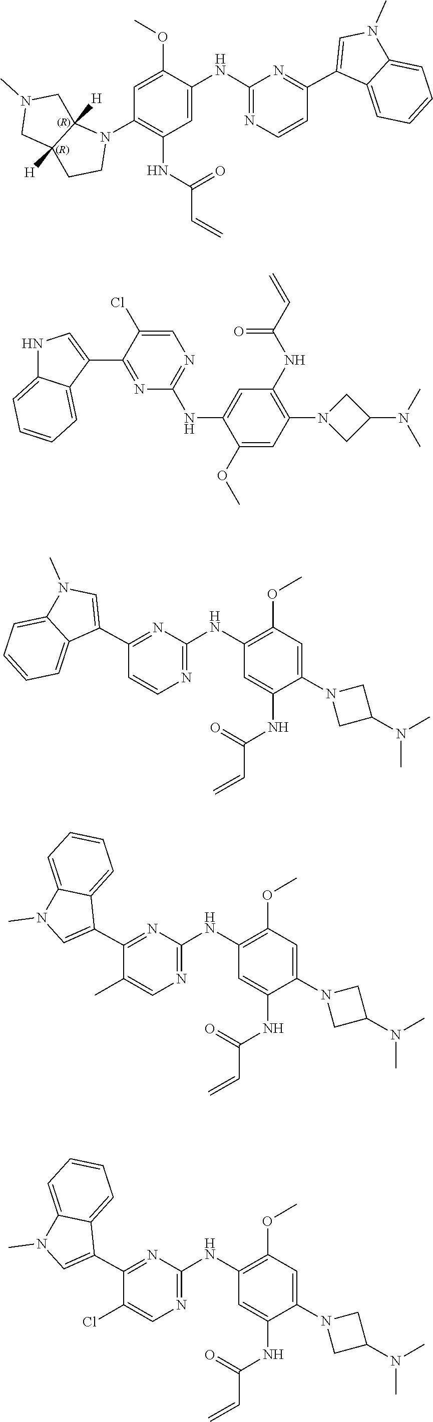 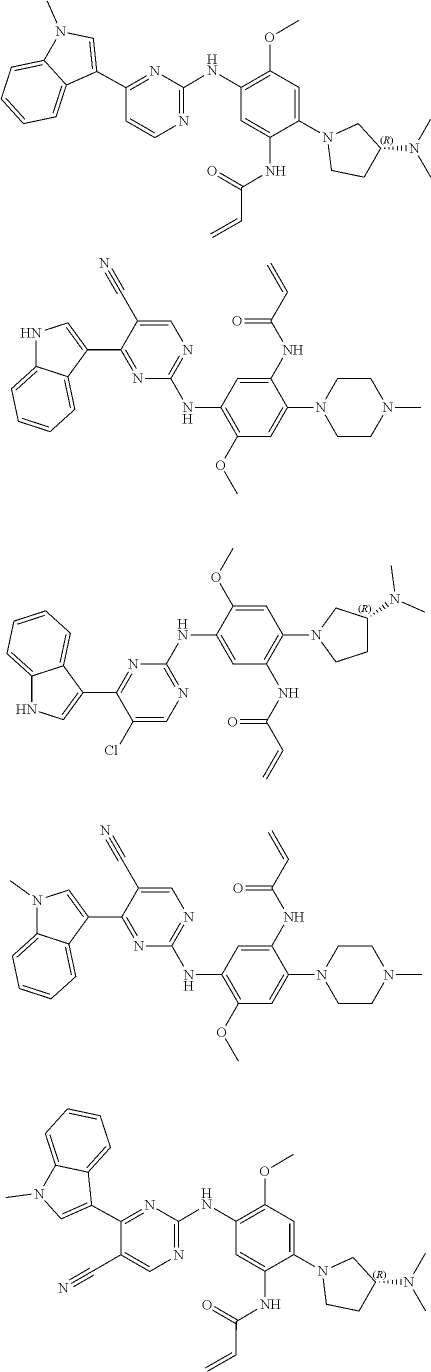 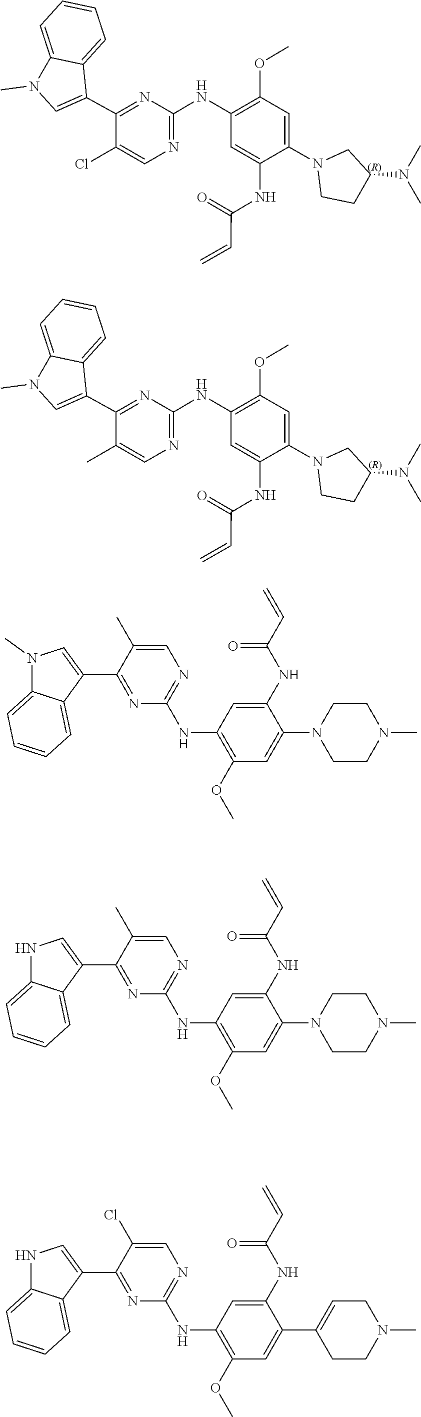 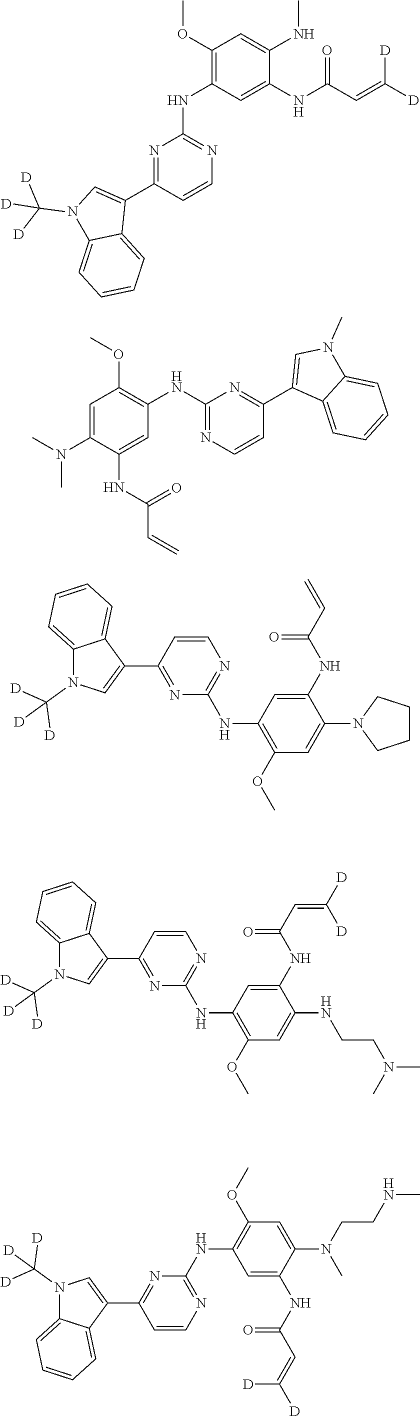 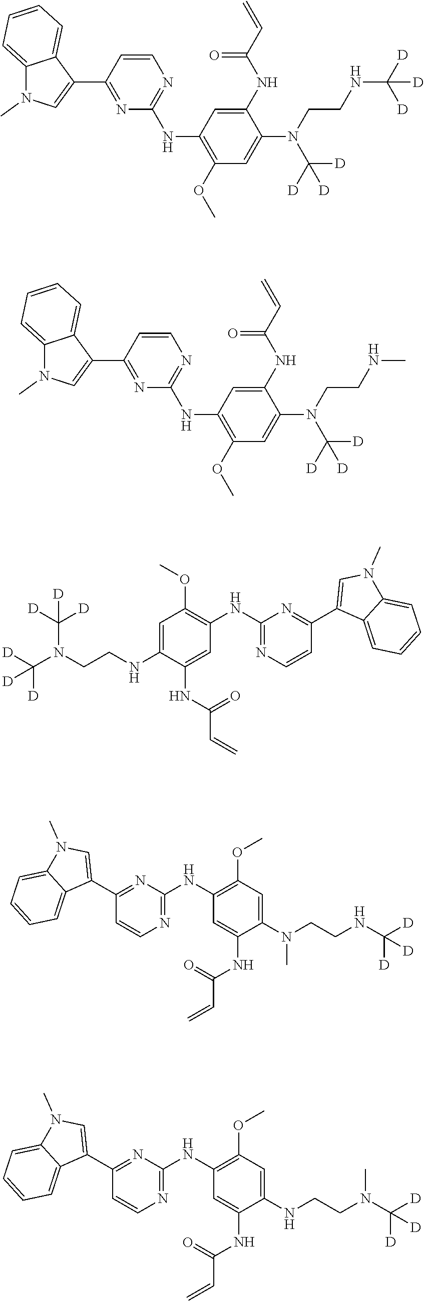 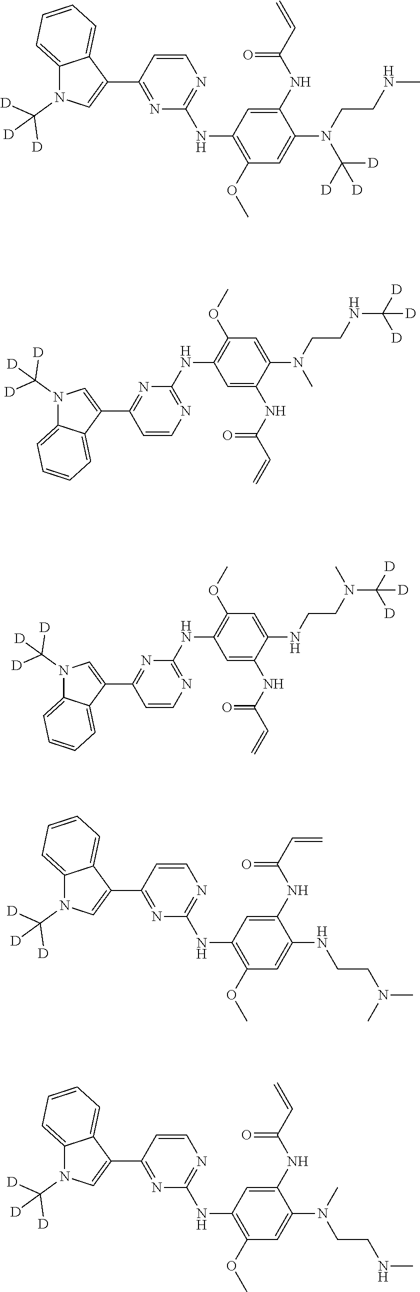 [SMILES: [2H]C([2H])([2H])N(CCNC)c1cc(OC)c(Nc2nccc(-c3cn(C([2H])([2H])[2H])c4ccccc34)n2)cc1NC(=O)C=C ; [2H]C([2H])([2H])NCCN(C)c1cc(OC)c(Nc2nccc(-c3cn(C([2H])([2H])[2H])c4ccccc34)n2)cc1NC(=O)C=C ; [2H]C([2H])([2H])N(C)CCNc1cc(OC)c(Nc2nccc(-c3cn(C([2H])([2H])[2H])c4ccccc34)n2)cc1NC(=O)C=C ; [2H]C([2H])([2H])n1cc(-c2ccnc(Nc3cc(NC(=O)C=C)c(NCCN(C)C)cc3OC)n2)c2ccccc21 ; [2H]C([2H])([2H])n1cc(-c2ccnc(Nc3cc(NC(=O)C=C)c(N(C)CCNC)cc3OC)n2)c2ccccc21] 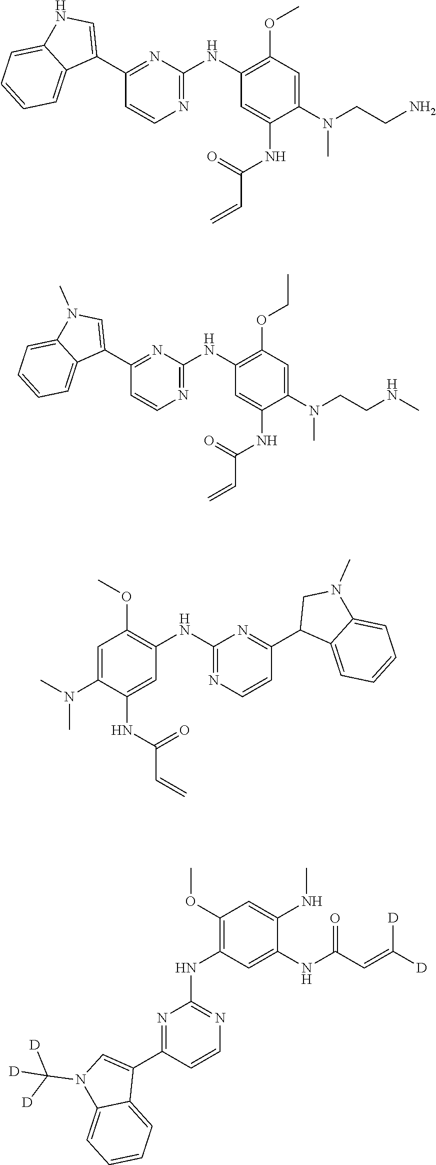 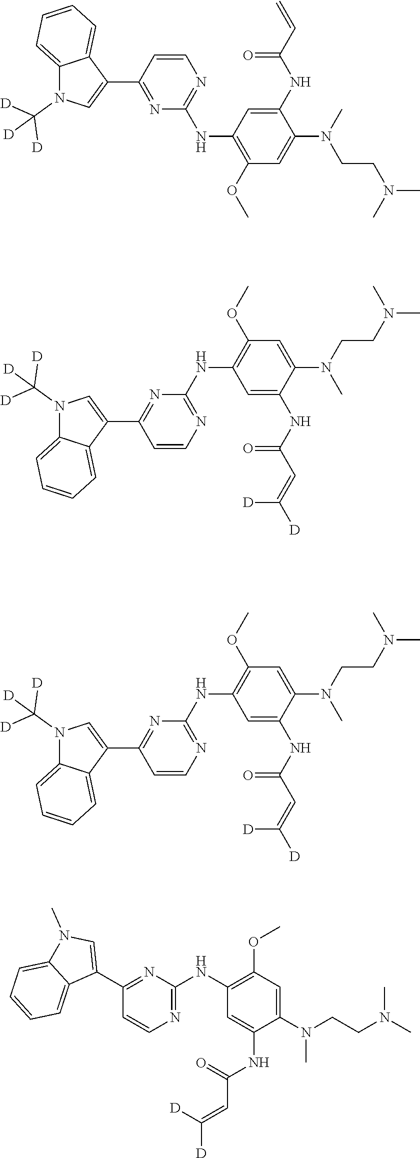 [SMILES: [2H]C([2H])([2H])n1cc(-c2ccnc(Nc3cc(NC(=O)C=C)c(N(C)CCN(C)C)cc3OC)n2)c2ccccc21 ; [2H]C([2H])=CC(=O)Nc1cc(Nc2nccc(-c3cn(C([2H])([2H])[2H])c4ccccc34)n2)c(OC)cc1N(C)CCN(C)C ; [2H]C([2H])=CC(=O)Nc1cc(Nc2nccc(-c3cn(C([2H])([2H])[2H])c4ccccc34)n2)c(OC)cc1N(C)CCN(C)C ; [2H]C([2H])=CC(=O)Nc1cc(Nc2nccc(-c3cn(C)c4ccccc34)n2)c(OC)cc1N(C)CCN(C)C] 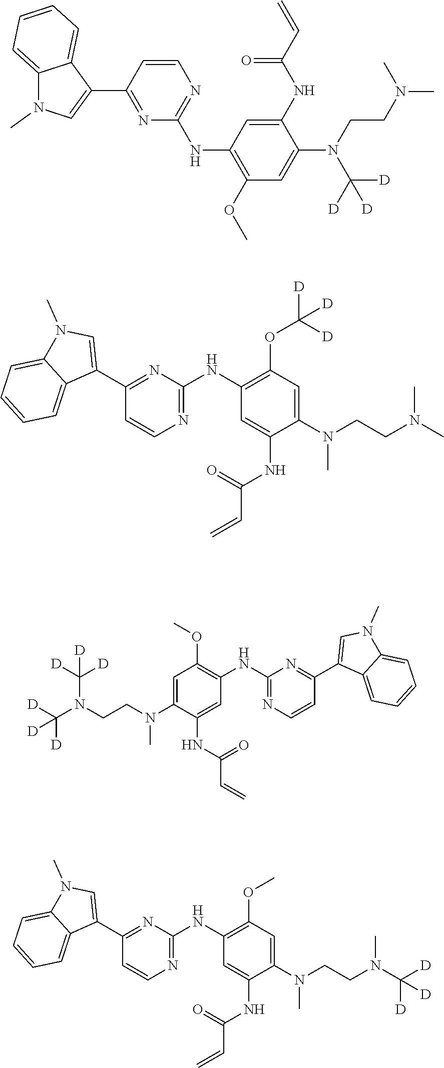
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope;
one or more fluoro atoms are replaced by the corresponding ¹⁸F isotope;
or a pharmaceutically acceptable salt thereof.

3. A compound according to formula (Ia or Ib) 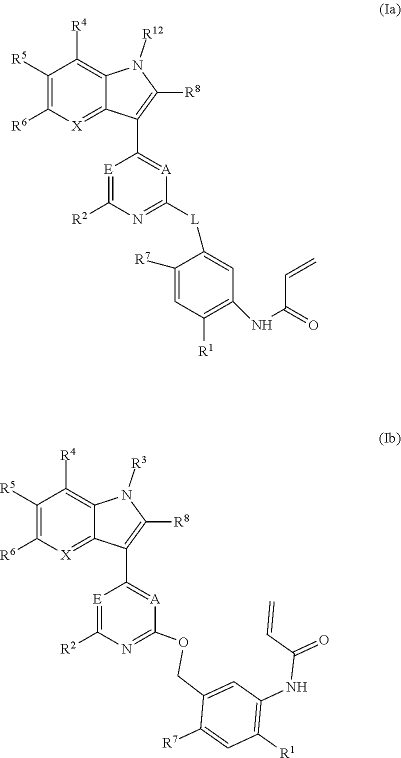
wherein
A is N or C—R; preferably C—R, more preferably CH;
E is N or C—R; preferably C—R, more preferably CH;
X is N, C—H, preferably CH;
R is hydrogen, fluoro, chloro, bromo, —(C₁-C₆)alkyl, cyano, —O(C₁-C₆)alkyl, preferably hydrogen;
L is 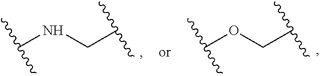
preferably 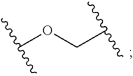 [SMILES: *CO*]
R¹ is selected from a group of alkylamines, non-aromatic heterocycles, partially or wholly deuterated alkylamines, and partially or wholly deuterated non-aromatic heterocycles including 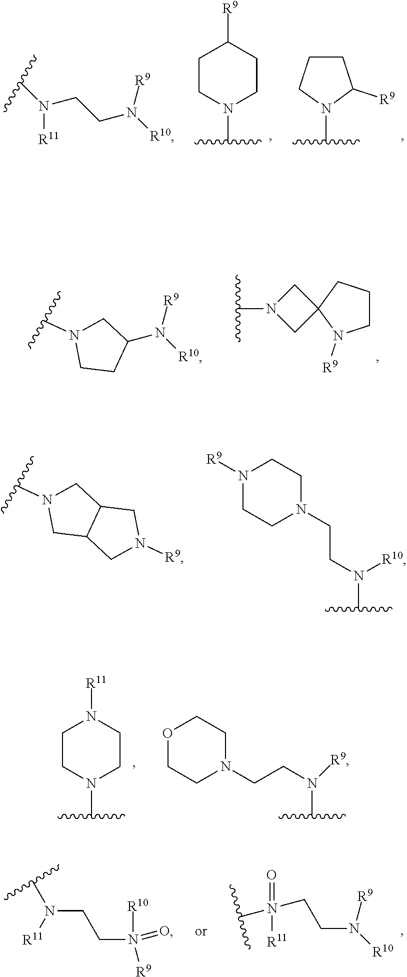
preferably 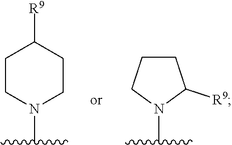
R² is hydrogen, fluoro, chloro, bromo, cyano, —(C₁-C₆)alkyl, preferably hydrogen;
R³ is hydrogen, —(C₁-C₆)alkyl, —(C₃-C₆)cycloalkyl, preferably methyl;
R⁴ is hydrogen, —(C₁-C₆)alkyl, preferably hydrogen;
R⁵ is hydrogen, —(C₁-C₆)alkyl, preferably hydrogen;
R⁶ is hydrogen, —(C₁-C₆)alkyl, preferably hydrogen;
R⁷ is hydrogen, fluoro, —O(C₁-C₆)alkyl, —(C₁-C₆)alkyl, preferably hydrogen;
R⁸ is hydrogen, —(C₁-C₆)alkyl, preferably hydrogen;
R⁹ is hydrogen, —(C₁-C₆)alkyl, preferably —(C₁-C₆)alkyl;
R¹⁰ is hydrogen, —(C₁-C₆)alkyl, preferably —(C₁-C₆)alkyl;
R¹¹ is hydrogen, —(C₁-C₆)alkyl, preferably —(C₁-C₆)alkyl;
R¹² is —(C₁-C₆)alkyl, —(C₃-C₆)cycloalkyl, preferably —(C₁-C₆)alkyl:
with the provision that A=N and 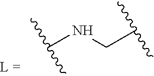 [SMILES: *CN*]
are not simultaneously present in the same compound.
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope;
one or more fluoro atoms are replaced by the corresponding ¹⁸F isotope.

4. The compound for use of claim 1 or the compound of claim 2 wherein in formula (I)
G is 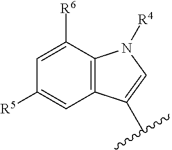
X is selected from N;
Y is selected from N;
A is selected from N or C—R;
E is selected from N or C—R¹
R is hydrogen;
R¹ is hydrogen;
R² is methoxy;
R³ is 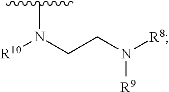
R⁴ is hydrogen, or —(C₁-C₆)alkyl;
R⁵ is hydrogen;
R⁶ is hydrogen;
R³ is hydrogen, or —(C₁-C₆)alkyl, preferably —(C₁-C₆)alkyl;
R⁹ is hydrogen, or —(C₁-C₆)alkyl, preferably —(C₁-C₆)alkyl;
R¹⁰ is hydrogen, or —(C₁-C₆)alkyl;

5. The compound for use of claim 1, or the compound of claim 2 wherein in formula (I),
G is 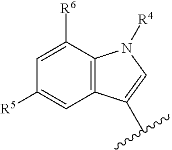
X is selected from N;
Y is selected from N;
A is selected from N or C—R;
E is selected from N or C—R;
R is hydrogen;
R¹ is hydrogen;
R² is methoxy;
R³ is 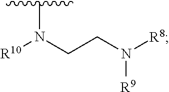
R⁴ is methyl;
R⁵ is hydrogen;
R⁶ is hydrogen;
R⁸ is hydrogen, or methyl;
R⁹ is methyl;
R¹⁰ is methyl;
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope;
one or more fluoro atoms are replaced by the corresponding ¹⁸F isotope;
or a pharmaceutically acceptable salt thereof.

6. The compound for use of claims 1, 4, 5 and the compound of claim 2, 4 and 5, wherein only one or two of A, E, X, and Y is N; only one of A and E is N, and only one of A and Y is N.

7. The compound for use of claims 1, and 3, wherein the compound is selected from the group consisting of 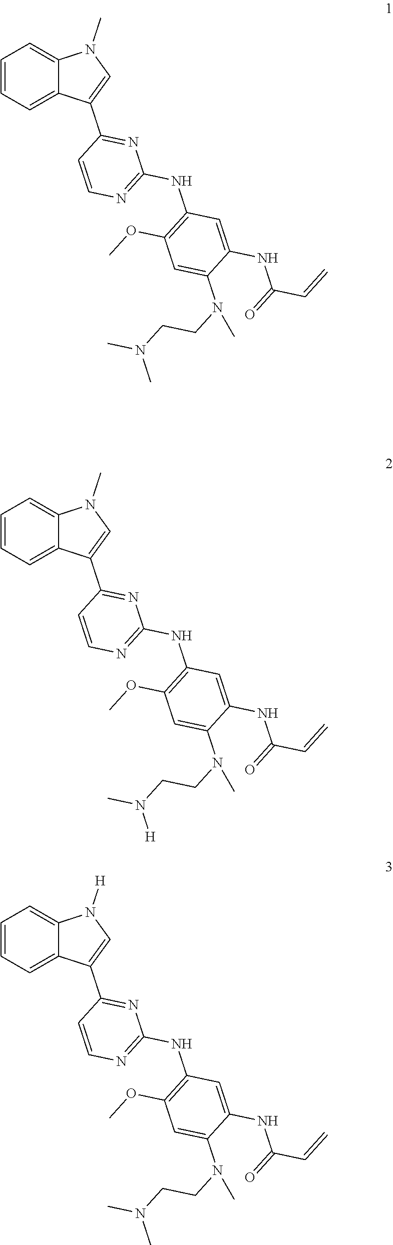 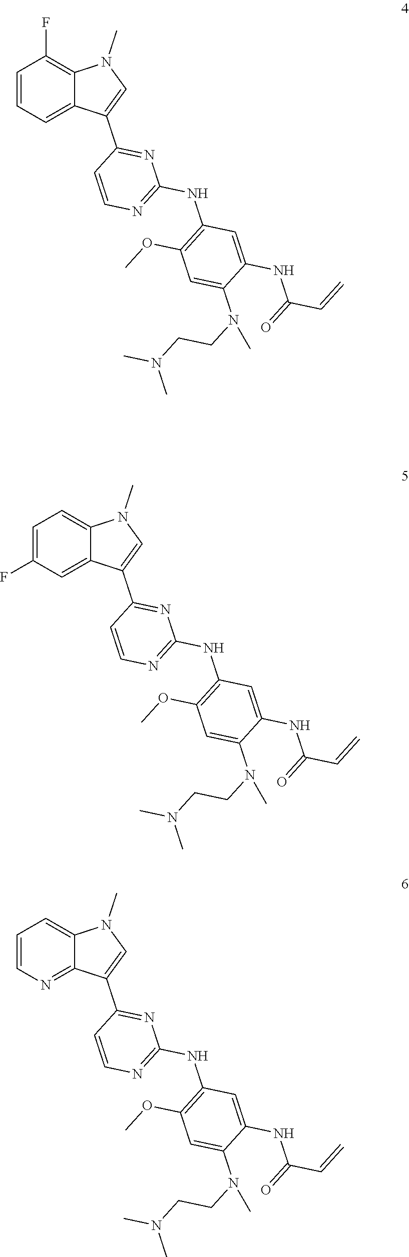 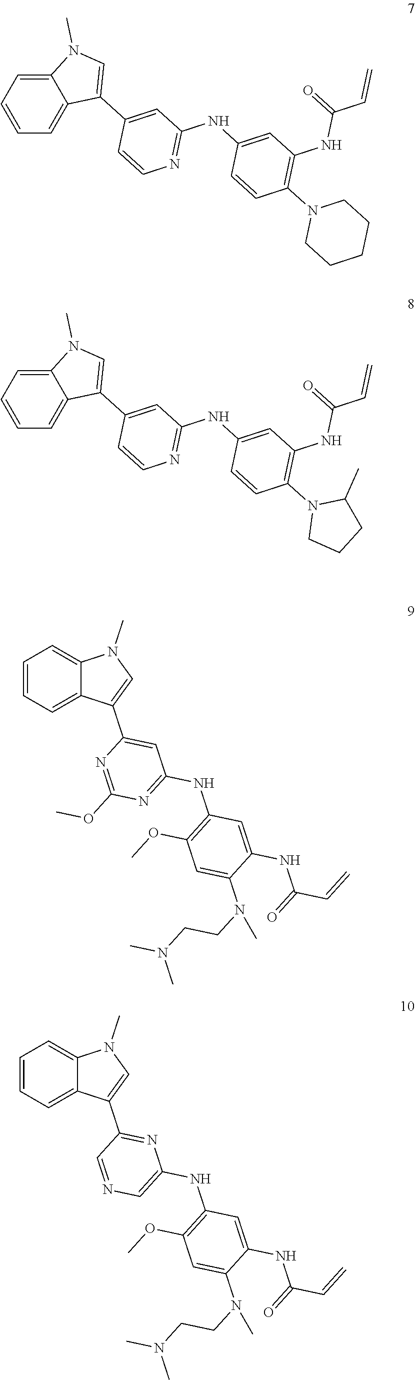 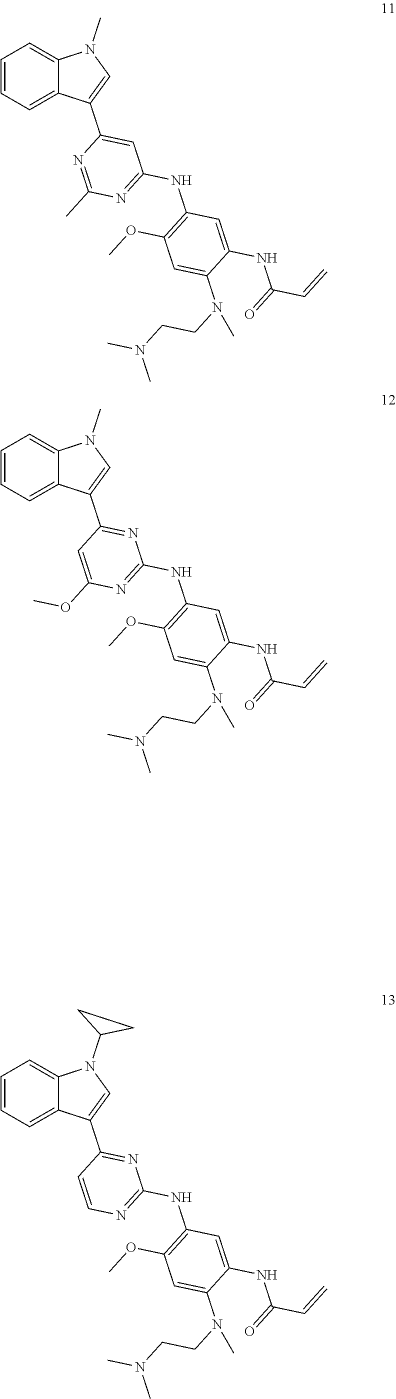 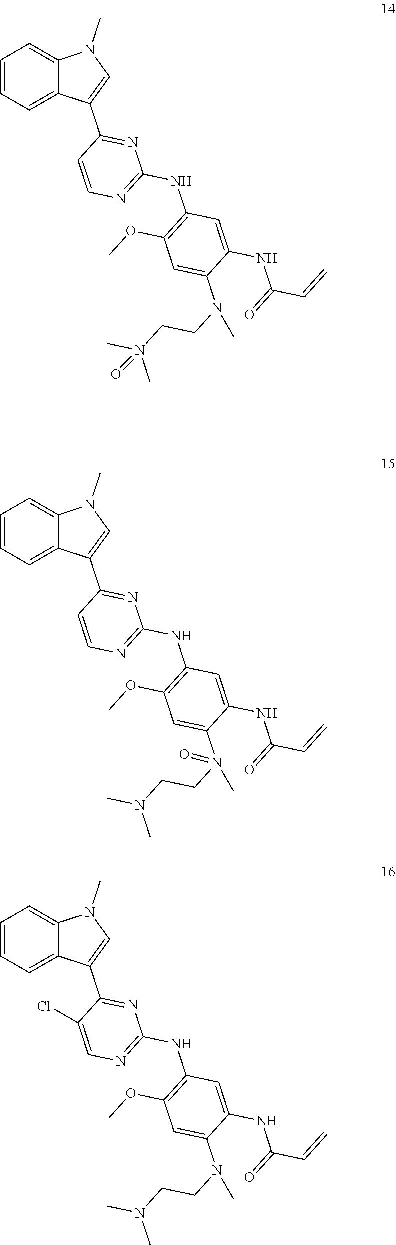 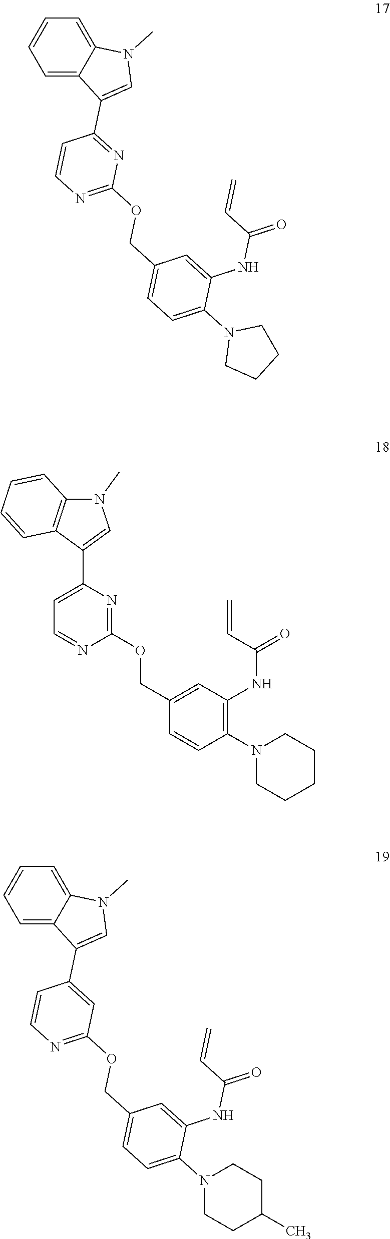 [SMILES: C=CC(=O)Nc1cc(COc2nccc(-c3cn(C)c4ccccc34)n2)ccc1N1CCCC1 ; C=CC(=O)Nc1cc(COc2nccc(-c3cn(C)c4ccccc34)n2)ccc1N1CCCCC1 ; C=CC(=O)Nc1cc(COc2cc(-c3cn(C)c4ccccc34)ccn2)ccc1N1CCC(C)CC1] 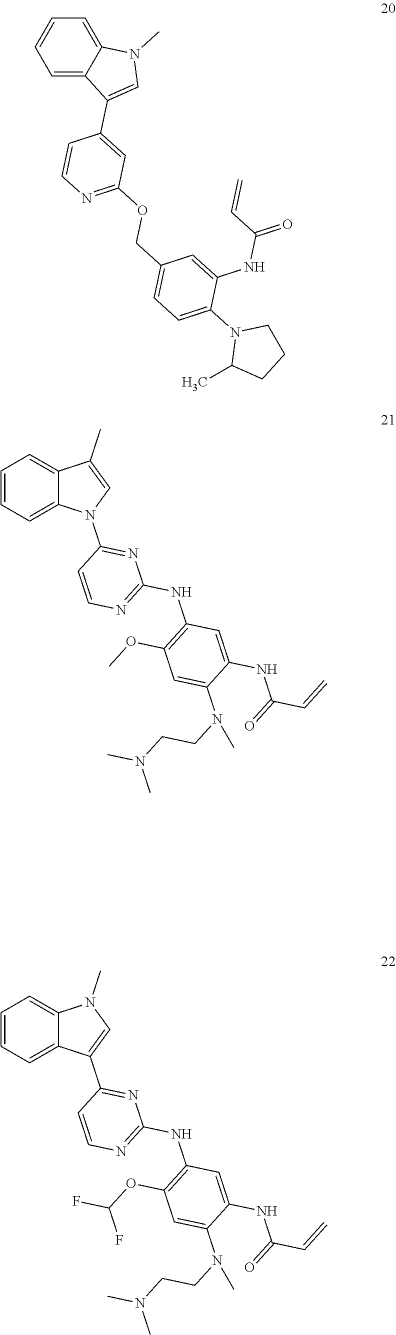 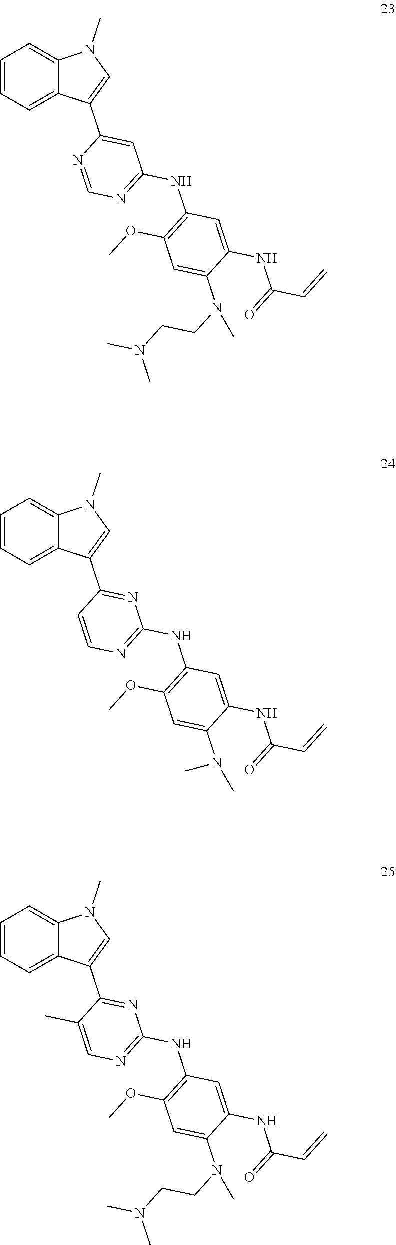 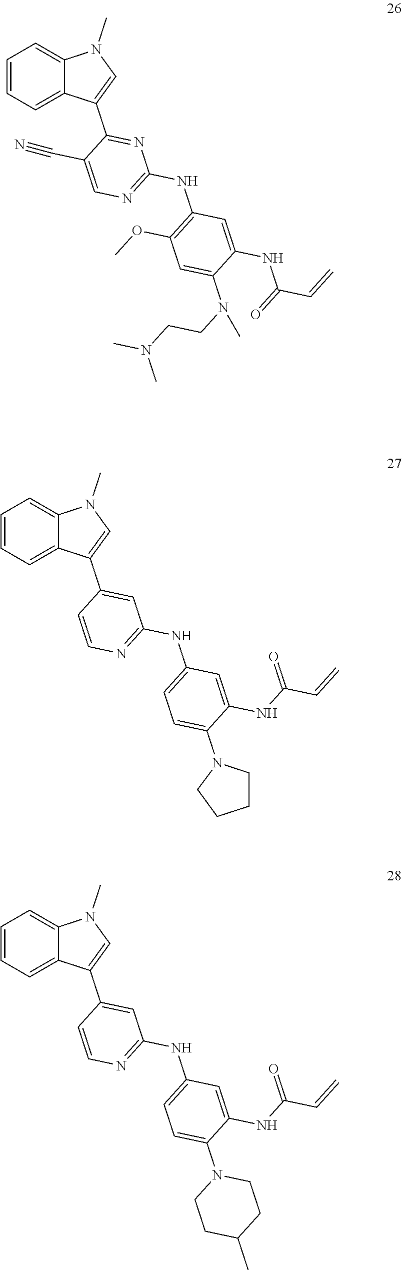 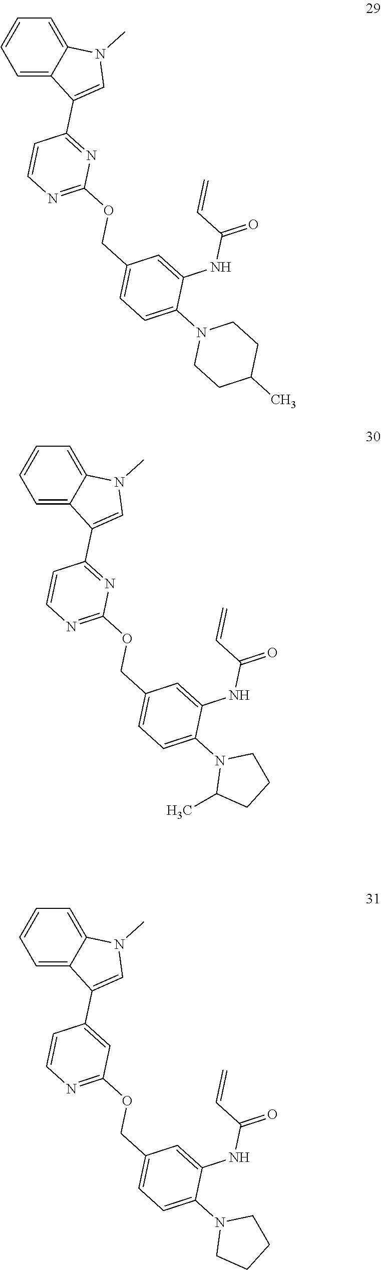 [SMILES: C=CC(=O)Nc1cc(COc2nccc(-c3cn(C)c4ccccc34)n2)ccc1N1CCC(C)CC1 ; C=CC(=O)Nc1cc(COc2nccc(-c3cn(C)c4ccccc34)n2)ccc1N1CCCC1C ; C=CC(=O)Nc1cc(COc2cc(-c3cn(C)c4ccccc34)ccn2)ccc1N1CCCC1] 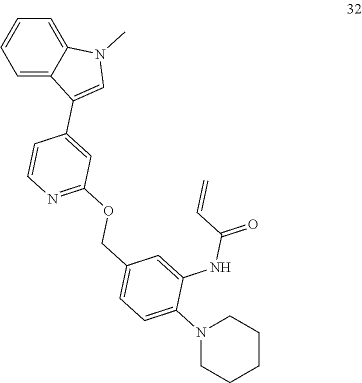 [SMILES: C=CC(=O)Nc1cc(COc2cc(-c3cn(C)c4ccccc34)ccn2)ccc1N1CCCCC1]
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope;
one or more fluoro atoms are replaced by the corresponding ¹⁸F isotope;
or a pharmaceutically acceptable salt thereof.

8. The compound for use of claim 7, wherein the compound is selected from the group consisting of 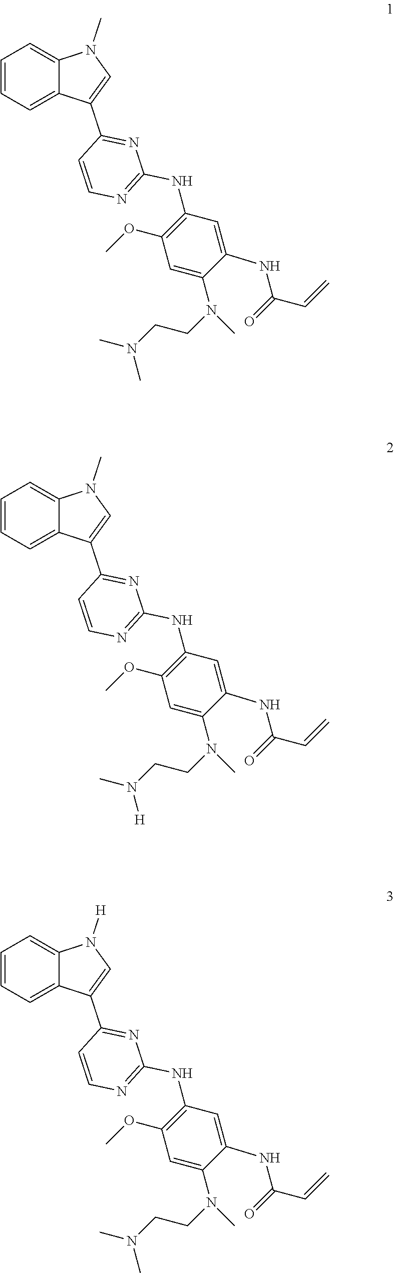 [SMILES: C=CC(=O)Nc1cc(Nc2nccc(-c3cn(C)c4ccccc34)n2)c(OC)cc1N(C)CCN(C)C ; C=CC(=O)Nc1cc(Nc2nccc(-c3cn(C)c4ccccc34)n2)c(OC)cc1N(C)CCNC ; C=CC(=O)Nc1cc(Nc2nccc(-c3c[nH]c4ccccc34)n2)c(OC)cc1N(C)CCN(C)C]
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope.

9. A compound selected from the group consisting of 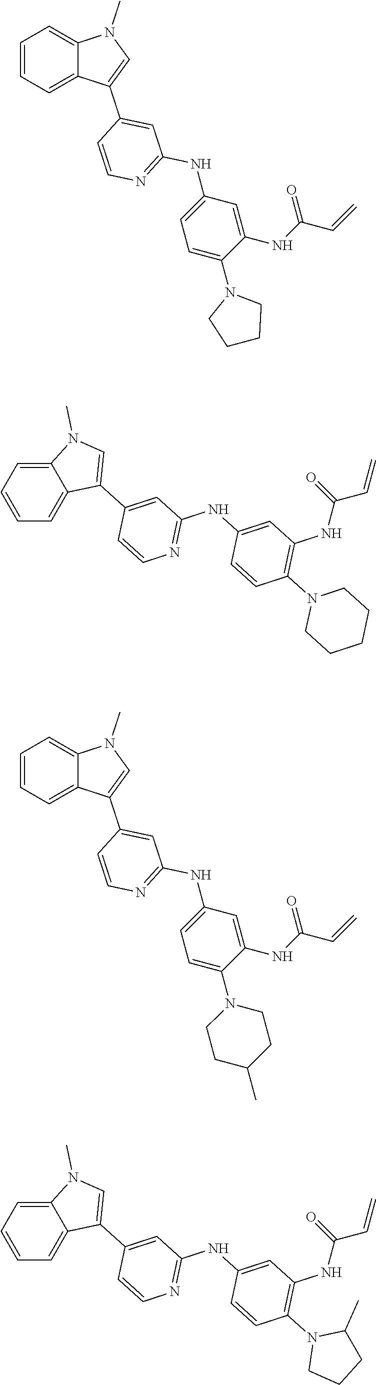 [SMILES: C=CC(=O)Nc1cc(Nc2cc(-c3cn(C)c4ccccc34)ccn2)ccc1N1CCCC1 ; C=CC(=O)Nc1cc(Nc2cc(-c3cn(C)c4ccccc34)ccn2)ccc1N1CCCCC1 ; C=CC(=O)Nc1cc(Nc2cc(-c3cn(C)c4ccccc34)ccn2)ccc1N1CCC(C)CC1 ; C=CC(=O)Nc1cc(Nc2cc(-c3cn(C)c4ccccc34)ccn2)ccc1N1CCCC1C] 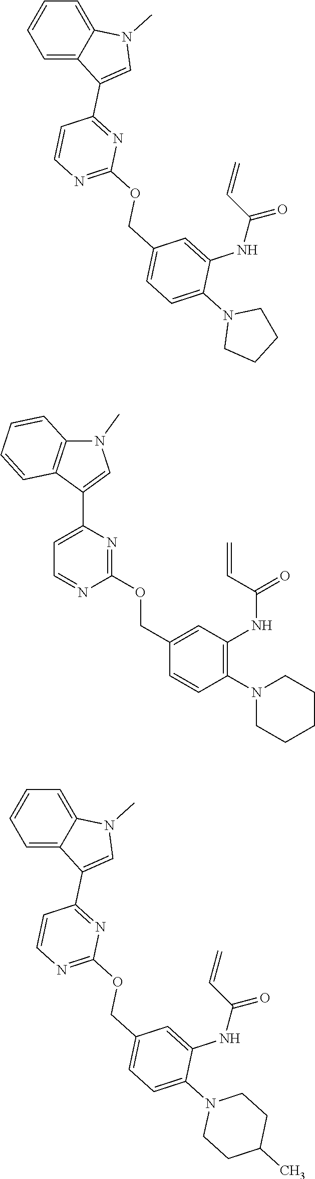 [SMILES: C=CC(=O)Nc1cc(COc2nccc(-c3cn(C)c4ccccc34)n2)ccc1N1CCCC1 ; C=CC(=O)Nc1cc(COc2nccc(-c3cn(C)c4ccccc34)n2)ccc1N1CCCCC1 ; C=CC(=O)Nc1cc(COc2nccc(-c3cn(C)c4ccccc34)n2)ccc1N1CCC(C)CC1] 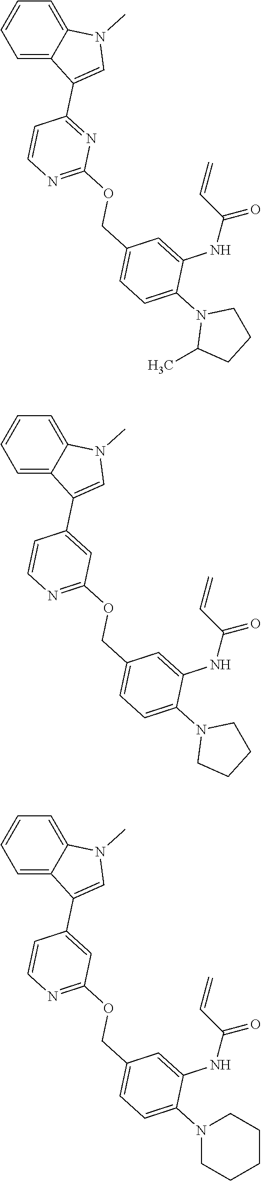 [SMILES: C=CC(=O)Nc1cc(COc2nccc(-c3cn(C)c4ccccc34)n2)ccc1N1CCCC1C ; C=CC(=O)Nc1cc(COc2cc(-c3cn(C)c4ccccc34)ccn2)ccc1N1CCCC1 ; C=CC(=O)Nc1cc(COc2cc(-c3cn(C)c4ccccc34)ccn2)ccc1N1CCCCC1] 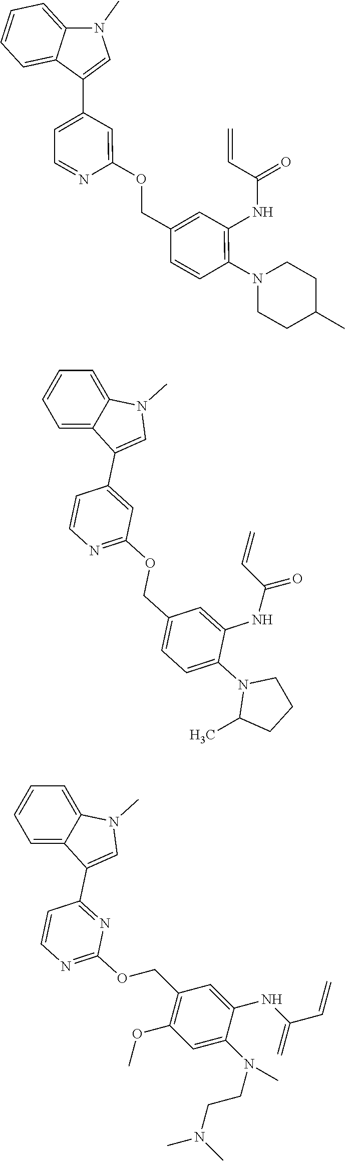 [SMILES: C=CC(=O)Nc1cc(COc2cc(-c3cn(C)c4ccccc34)ccn2)ccc1N1CCC(C)CC1 ; C=CC(=O)Nc1cc(COc2cc(-c3cn(C)c4ccccc34)ccn2)ccc1N1CCCC1C ; C=CC(=C)Nc1cc(COc2nccc(-c3cn(C)c4ccccc34)n2)c(OC)cc1N(C)CCN(C)C] 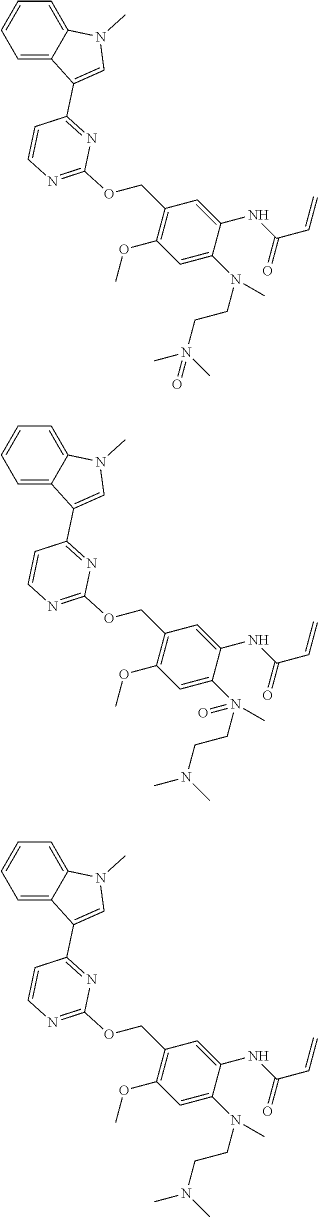 [SMILES: C=CC(=O)Nc1cc(COc2nccc(-c3cn(C)c4ccccc34)n2)c(OC)cc1N(C)CC[N+](C)(C)[O-] ; C=CC(=O)Nc1cc(COc2nccc(-c3cn(C)c4ccccc34)n2)c(OC)cc1[N+](C)([O-])CCN(C)C ; C=CC(=O)Nc1cc(COc2nccc(-c3cn(C)c4ccccc34)n2)c(OC)cc1N(C)CCN(C)C] 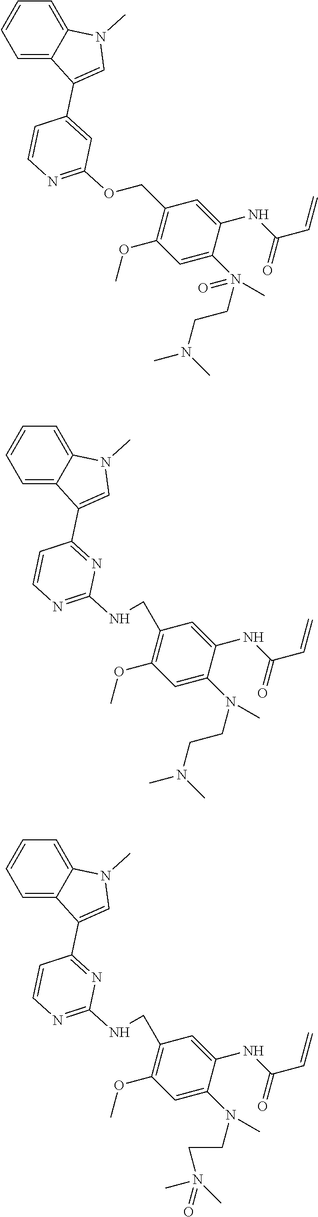 [SMILES: C=CC(=O)Nc1cc(COc2cc(-c3cn(C)c4ccccc34)ccn2)c(OC)cc1[N+](C)([O-])CCN(C)C ; C=CC(=O)Nc1cc(CNc2nccc(-c3cn(C)c4ccccc34)n2)c(OC)cc1N(C)CCN(C)C ; C=CC(=O)Nc1cc(CNc2nccc(-c3cn(C)c4ccccc34)n2)c(OC)cc1N(C)CC[N+](C)(C)[O-]] 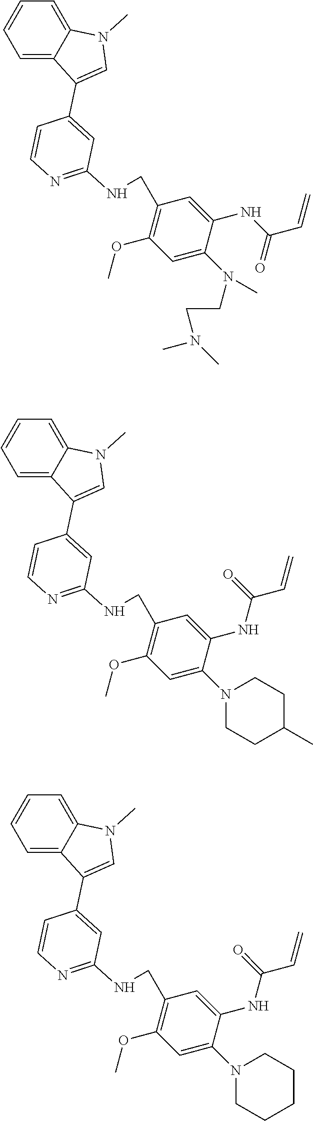 [SMILES: C=CC(=O)Nc1cc(CNc2cc(-c3cn(C)c4ccccc34)ccn2)c(OC)cc1N(C)CCN(C)C ; C=CC(=O)Nc1cc(CNc2cc(-c3cn(C)c4ccccc34)ccn2)c(OC)cc1N1CCC(C)CC1 ; C=CC(=O)Nc1cc(CNc2cc(-c3cn(C)c4ccccc34)ccn2)c(OC)cc1N1CCCCC1] 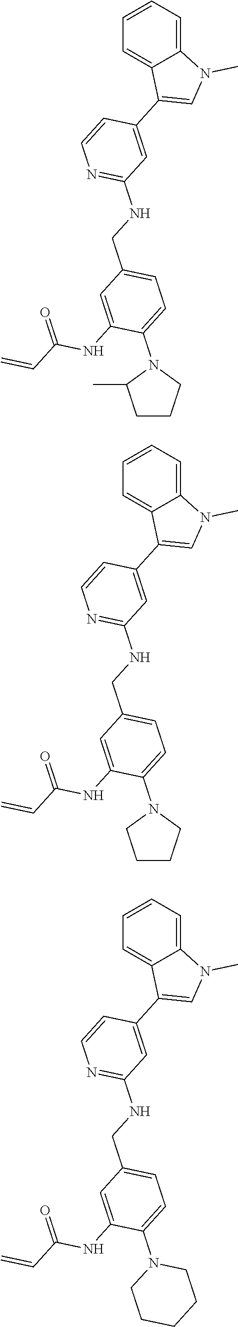 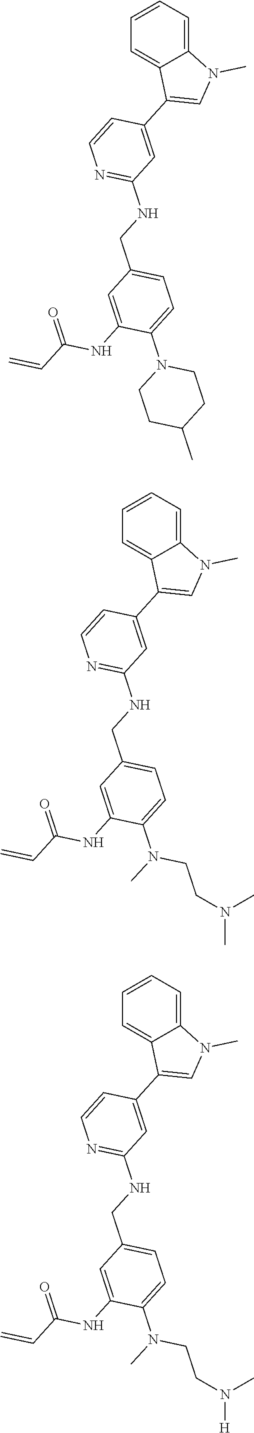 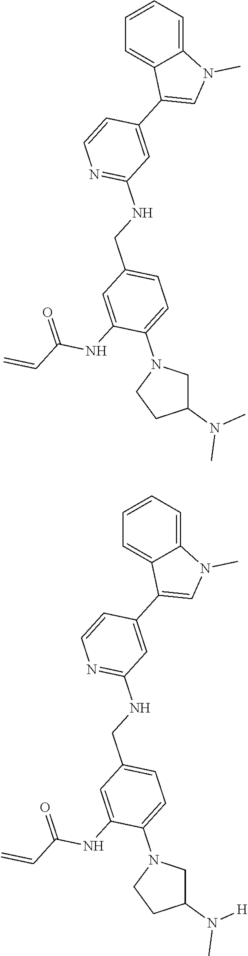 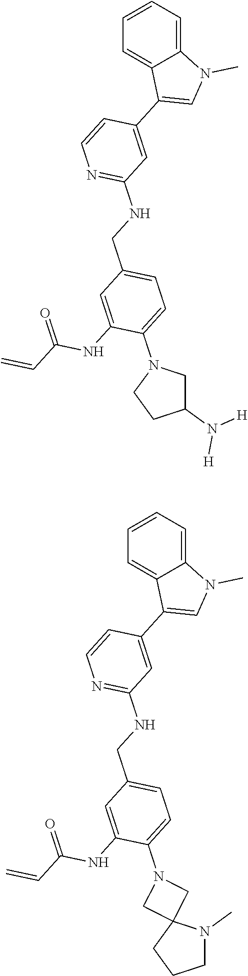 [SMILES: C=CC(=O)Nc1cc(CNc2cc(-c3cn(C)c4ccccc34)ccn2)ccc1N1CCC(N)C1 ; C=CC(=O)Nc1cc(CNc2cc(-c3cn(C)c4ccccc34)ccn2)ccc1N1CC2(CCCN2C)C1] 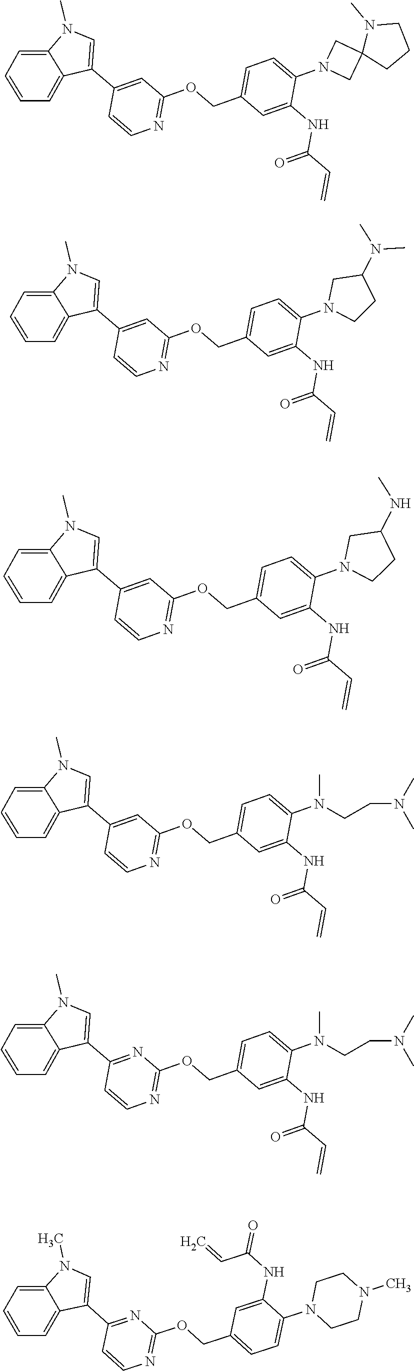 [SMILES: C=CC(=O)Nc1cc(COc2cc(-c3cn(C)c4ccccc34)ccn2)ccc1N1CC2(CCCN2C)C1 ; C=CC(=O)Nc1cc(COc2cc(-c3cn(C)c4ccccc34)ccn2)ccc1N1CCC(N(C)C)C1 ; C=CC(=O)Nc1cc(COc2cc(-c3cn(C)c4ccccc34)ccn2)ccc1N1CCC(NC)C1 ; C=CC(=O)Nc1cc(COc2cc(-c3cn(C)c4ccccc34)ccn2)ccc1N(C)CCN(C)C ; C=CC(=O)Nc1cc(COc2nccc(-c3cn(C)c4ccccc34)n2)ccc1N(C)CCN(C)C ; C=CC(=O)Nc1cc(COc2nccc(-c3cn(C)c4ccccc34)n2)ccc1N1CCN(C)CC1] 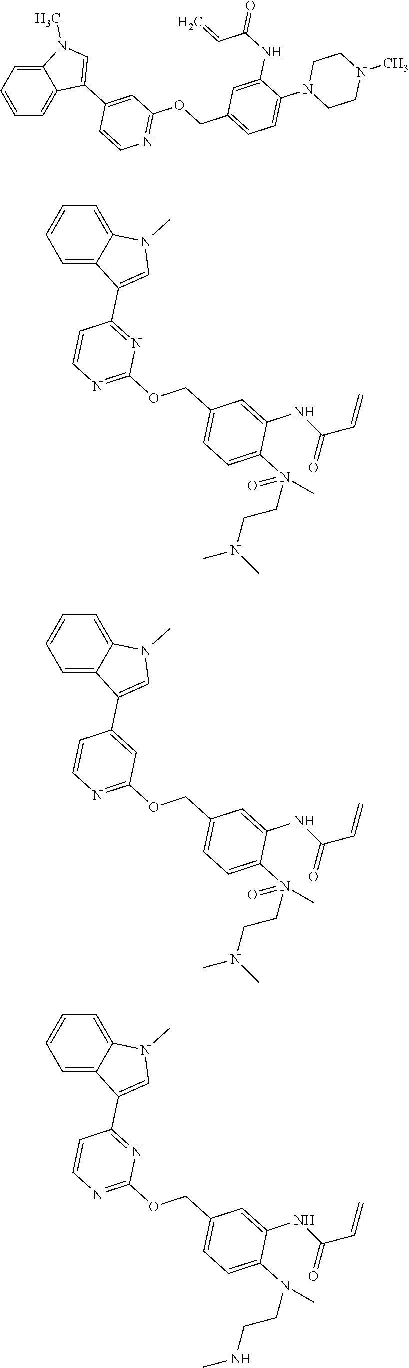 [SMILES: C=CC(=O)Nc1cc(COc2cc(-c3cn(C)c4ccccc34)ccn2)ccc1N1CCN(C)CC1 ; C=CC(=O)Nc1cc(COc2nccc(-c3cn(C)c4ccccc34)n2)ccc1[N+](C)([O-])CCN(C)C ; C=CC(=O)Nc1cc(COc2cc(-c3cn(C)c4ccccc34)ccn2)ccc1[N+](C)([O-])CCN(C)C ; C=CC(=O)Nc1cc(COc2nccc(-c3cn(C)c4ccccc34)n2)ccc1N(C)CCNC] 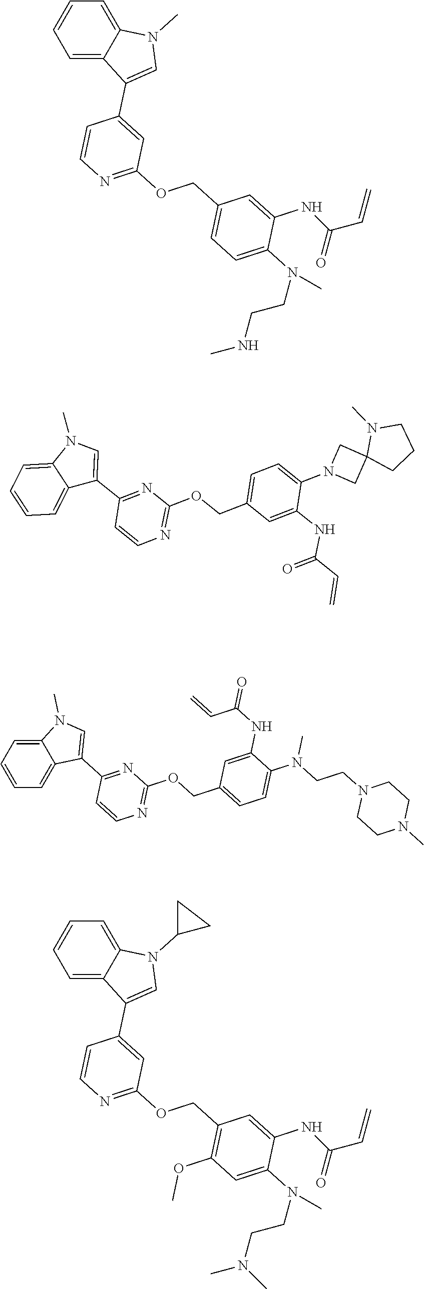 [SMILES: C=CC(=O)Nc1cc(COc2cc(-c3cn(C)c4ccccc34)ccn2)ccc1N(C)CCNC ; C=CC(=O)Nc1cc(COc2nccc(-c3cn(C)c4ccccc34)n2)ccc1N1CC2(CCCN2C)C1 ; C=CC(=O)Nc1cc(COc2nccc(-c3cn(C)c4ccccc34)n2)ccc1N(C)CCN1CCN(C)CC1 ; C=CC(=O)Nc1cc(COc2cc(-c3cn(C4CC4)c4ccccc34)ccn2)c(OC)cc1N(C)CCN(C)C] 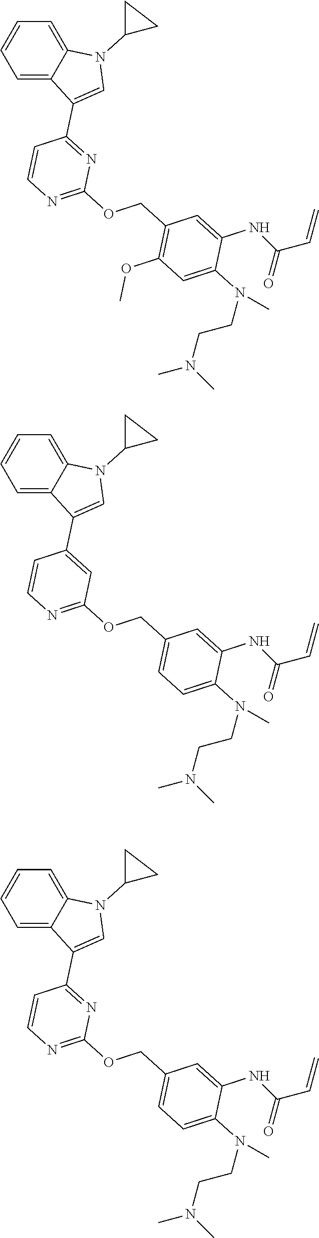 [SMILES: C=CC(=O)Nc1cc(COc2nccc(-c3cn(C4CC4)c4ccccc34)n2)c(OC)cc1N(C)CCN(C)C ; C=CC(=O)Nc1cc(COc2cc(-c3cn(C4CC4)c4ccccc34)ccn2)ccc1N(C)CCN(C)C ; C=CC(=O)Nc1cc(COc2nccc(-c3cn(C4CC4)c4ccccc34)n2)ccc1N(C)CCN(C)C] 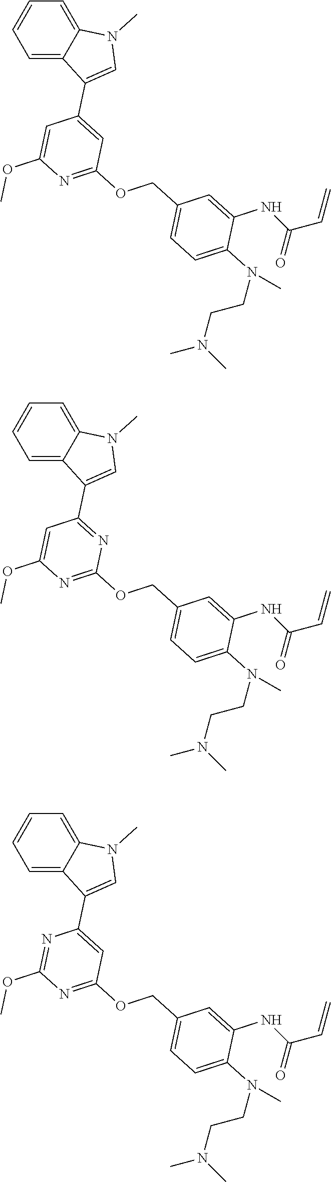 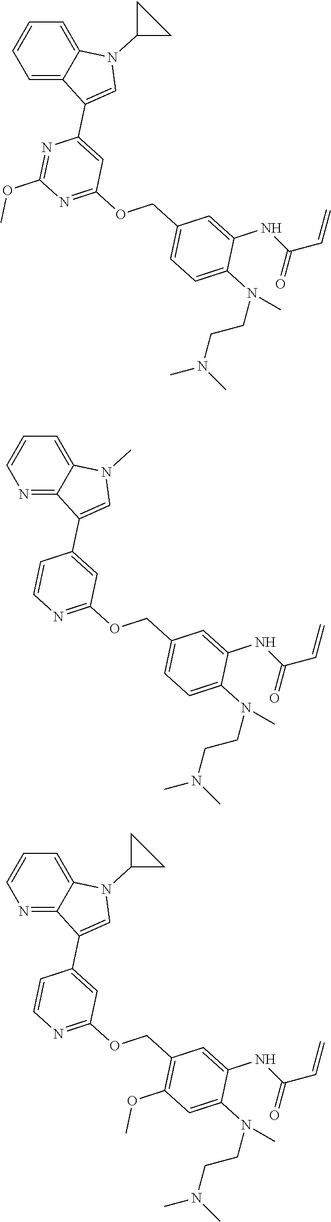 [SMILES: C=CC(=O)Nc1cc(COc2cc(-c3cn(C4CC4)c4ccccc34)nc(OC)n2)ccc1N(C)CCN(C)C ; C=CC(=O)Nc1cc(COc2cc(-c3cn(C)c4cccnc34)ccn2)ccc1N(C)CCN(C)C ; C=CC(=O)Nc1cc(COc2cc(-c3cn(C4CC4)c4cccnc34)ccn2)c(OC)cc1N(C)CCN(C)C] 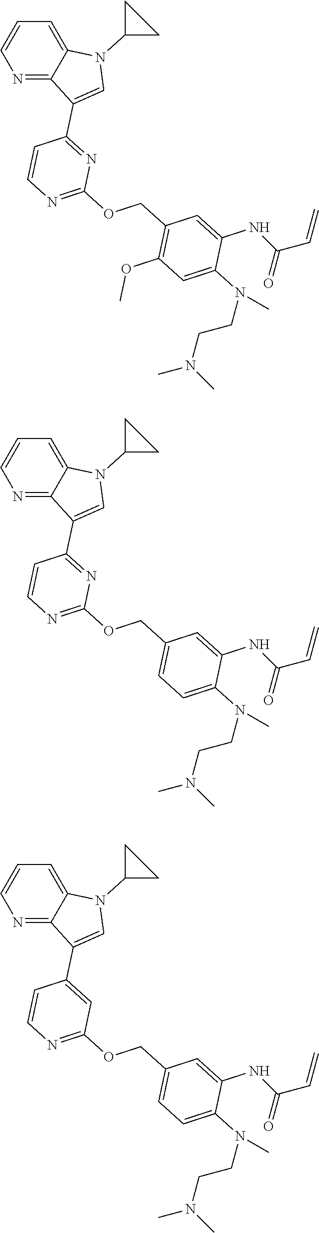 [SMILES: C=CC(=O)Nc1cc(COc2nccc(-c3cn(C4CC4)c4cccnc34)n2)c(OC)cc1N(C)CCN(C)C ; C=CC(=O)Nc1cc(COc2nccc(-c3cn(C4CC4)c4cccnc34)n2)ccc1N(C)CCN(C)C ; C=CC(=O)Nc1cc(COc2cc(-c3cn(C4CC4)c4cccnc34)ccn2)ccc1N(C)CCN(C)C] 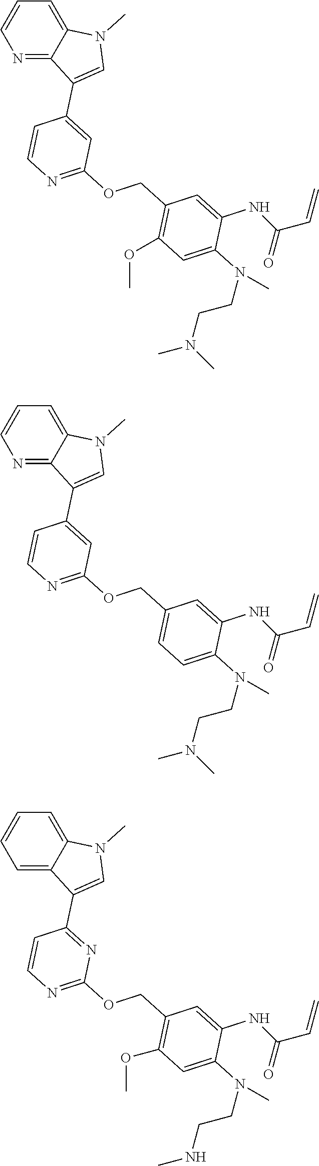 [SMILES: C=CC(=O)Nc1cc(COc2cc(-c3cn(C)c4cccnc34)ccn2)c(OC)cc1N(C)CCN(C)C ; C=CC(=O)Nc1cc(COc2cc(-c3cn(C)c4cccnc34)ccn2)ccc1N(C)CCN(C)C ; C=CC(=O)Nc1cc(COc2nccc(-c3cn(C)c4ccccc34)n2)c(OC)cc1N(C)CCNC] 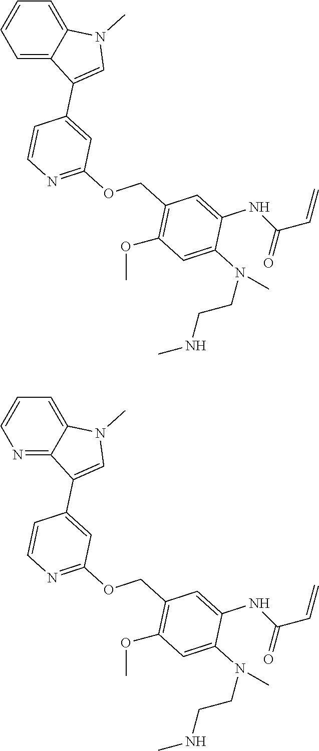 [SMILES: C=CC(=O)Nc1cc(COc2cc(-c3cn(C)c4ccccc34)ccn2)c(OC)cc1N(C)CCNC ; C=CC(=O)Nc1cc(COc2cc(-c3cn(C)c4cccnc34)ccn2)c(OC)cc1N(C)CCNC]
wherein optionally,
one or more hydrogen atoms are replaced by a deuterium atom;
one or more carbon atoms are replaced by the corresponding ¹¹C isotope;
one or more nitrogen atoms are replaced by the corresponding ¹³N isotope;
one or more fluoro atoms are replaced by the corresponding ¹⁸F-isotope;
or a pharmaceutically acceptable salt thereof.

10. A pharmaceutical composition comprising the compound according to formula (I), formula (Ia) or formula (Ib) in claims 1 to 9 and at least one pharmaceutically acceptable carrier for use in preventing or treating a tauopathy.

11. The compound for use of claims 1, and 4 to 8, or the pharmaceutical composition for use of claim 10 wherein preventing or treating a tauopathy comprises a causal treatment of the tauopathy, or the compound of claims 2, 3, 4 to 6 and 9 for use in preventing or treating a tauopathy comprising a causal treatment of a tauopathy.

12. The compound for use of claims 1, 4 to 8, 10, or the pharmaceutical composition for use or the compound of claim 10 or 11, wherein preventing or treating a tauopathy comprises inhibiting the cellular activity of tau by binding of the compound according to formula (I) to tau or the compound of claims 2, 3, 4 to 6 and 9 for use in preventing or treating a tauopathy comprising inhibiting the cellular activity of tau by binding of the compound according to formula (I), formula (Ia) or formula (Ib).

13. The compound for use of claims 1, 4 to 8, and 10 to 12, or the pharmaceutical composition for use or the compound of claims 10 to 12, wherein the binding to tau comprises covalent binding of the compound according to general formula (I) to tau, preferably the binding comprises a thiol-Michael addition of an —SH group of tau to the acrylamide group in the compound according to formula (I) or the compound of claims 2, 3, 4 to 6 and 9 for use in preventing or treating a tauopathy comprising inhibiting the cellular activity of tau by binding of the compound according to formula (I), formula (Ia) or formula (Ib), wherein the binding to tau comprises covalent binding of the compound according to general formula (I) to tau, preferably the binding comprises a thiol-Michael addition of an —SH group of tau to the acrylamide group in the compound according to formula (I), formula (Ia) or formula (Ib).

14. The compound for use of claims 1, 4 to 8 and 10 to 13, or the pharmaceutical composition for use or the compound of claims 10 to 13 wherein the tauopathy is dementia-associated tauopathy or the compound of claims 2, 3, 4 to 6 and 9 for use in preventing or treating a dementia-associated tauopathy.

15. The compound for use of claims 1, 4 to 8, and 10 to 14, or the pharmaceutical composition for use or the compound of claims 10 to 14 or the compound of claims 2, 3, 4 to 6 and 9 for use in preventing or treating a tauopathy, wherein tauopathy is selected from the group consisting of Alzheimer's disease, frontotemporal dementia, primary age-related tauopathy (PART), familial British dementia (FBD), familial Danish dementia (FDD), chronic traumatic encephalopathy (CTE), progressive supranuclear palsy (PSP), corticobasal degeneration (CBD), Pick's disease (PiD), dementia with Lewy Bodies (DLB), progressive supranuclear palsy (PSP), globular glial tauopathy (GGT), tauopathy with hippocampal 4-repeat tau immunoreactive spherical inclusions, limbic-predominant neuronal inclusion body 4R tauopathy (LNT), frontotemporal dementia and parkinsonism linked to chromosome 17 (FTDP-17), argyrophilic grain disease (AGD), Huntington disease, glial globular tauopathy, neuro-astroglial tauopathy variants in the elderly, familial behavioural variant frontotemporal dementia associated with astrocyte-predominant tauopathy, amyotrophic lateral sclerosis (ALS), spinocerebellar ataxia type 11, spinal muscular atrophy (SMA), progressive ataxia and palatal tremor-related tauopathy, cerebral amyloid angiopathy (CAA), IgLON5 antibody-related tauopathy, Chromosome 21 trisomy-related Alzheimer's disease (Down's syndrome), vascular dementia, cerebral amyloid angiopathy, Gerstmann-Straussler-Scheinker disease (GSS), Creutzfeldt-Jakob disease, fatal familial insomnia, Kuru, Niemann-Pick disease type C-related tauopathy, Nodding syndrome, Non-Guamanian motor neuron disease with neurofibrillary tangles, Parkinson's disease (PD), Parkinson's disease with dementia, Parkinsonism-dementia of Guam, Guadeloupean parkinsonism, Kosaka-Shibayama disease, post-encephalitic parkinsonism, SYNJ1 (PARK20) early-onset recessive form of parkinsonism with seizures and dystonia associated nigral tau pathology, multiple system atrophy, tau pathology associated with familial parkinsonism and progressive respiratory failure, limbic predominant neuronal-glial tau pathology in TARDBP gene mutations (1383; P112H), neuronal 4R tau pathology in fatal familial insomnia (PRNP D178N mutation), tau pathology associated with ADCY5 dyskinesia, tau pathology in chronic temporal lobe epilepsy, neurodegeneration with brain iron accumulation (NBIA), tau pathology in NBIA PANK2 and WDR45 gene mutations, tau pathology in NBIA PLA2G6 mutation, tau pathology in NBIA associated with autosomal-dominant mitochondrial membrane protein-associated neurodegeneration (MPAN), neuropil threads pretangles and neurofibrillary tangles in human immunodeficiency virus (HIV)-negative opiate abusers, tau pathology associated with acquired immunodeficiency syndrome (AIDS), diffuse neurofibrillary tangles with calcification, progressive ataxia and palatal tremor, SLC9A6-related parkinsonism, tau pathology associated with SPG7 gene mutation, striatal 4R tau pathology associated to X-linked parkinsonism with spasticity (ATP6AP2), tau pathology associated with SPAST gene-related hereditary spastic paraplegia, autism, autism spectrum disorders, retinal tauopathy, West Nile encephalomyelitis, TTBK2 gene-related spinocerebellar ataxia 11, herpes simplex encephalitis, tangle-only dementia (TOD), aging-related tau astrogliopathy (ARTAG), hippocampal tauopathy, subacute sclerosing panencephalitis (SSPE)-related tauopathy, FTLD-C9ORF72, Christianson syndrome, vacuolar tauopathy, lytico-bodig disease, ganglioglioma and gangliocytoma, meningioangiomatosis, lead encephalopathy, tuberous sclerosis complex, pantothenate kinase-associated neurodegeneration, neuronal ceroid lipofuscinosis, myotonic dystrophy, Fukuyama congenital muscular dystrophy, hemimegalencephaly, focal cortical dysplasias, Wolcott-Rallison syndrome, primary lateral sclerosis, progressive gait freezing, PSP with parkinsonism, Richardson's syndrome, non-fluent/agrammatic variant of primary progressive aphasia, semantic variant of primary progressive aphasia, logopenic variant of primary progressive aphasia, primary progressive apraxia of speech, amnestic Alzheimer's disease,
preferably Alzheimer's disease (AD), Pick's disease (PiD), progressive supranuclear palsy (PSP), corticobasal degeneration (CBD), chronic traumatic encephalopathy (CTE), argyrophilic grain disease (AGD), frontotemporal dementia (FTD), amytrophic lateral sclerosis (ALS), Parkinson's disease (PD) and primary age-related tauopathy (PART).

16. The pharmaceutical composition for use according to claims 10 to 15, wherein the pharmaceutical composition is applied parenterally or orally, preferably orally.
